SEQUENCE LISTING (26 sequences)

SEQ ID NO: 1 - protein, 746 aa, Homo sapiens
MGQTGKKSEKGPVCWRKRVKSEYMRLRQLKRFRRADEVKSMFSSNRQKILERTEILNQEWKQRRIQPVHILTSVSSLRGTRECSVTSDLDFPTQVIPLKTLNAVASVPIMYSWSPLQQNFMVEDETVLHNIPYMGDEVLDQDGTFIEELIKNYDGKVHGDRECGFINDEIFVELVNALGQYNDDDDDDDGDDPEEREEKQKDLEDHRDDKESRPPRKFPSDKIFEAISSMFPDKGTAEELKEKYKELTEQQLPGALPPECTPNIDGPNAKSVQREQSLHSFHTLFCRRCFKYDCFLHPFHATPNTYKRKNTETALDNKPCGPQCYQHLEGAKEFAAALTAERIKTPPKRPGGRRRGRLPNNSSRPSTPTINVLESKDTDSDREAGTETGGENNDKEEEEKKDETSSSSEANSRCQTPIKMKPNIEPPENVEWSGAEASMFRVLIGTYYDNFCAIARLIGTKTCRQVYEFRVKESSIIAPAPAEDVDTPPRKKKRKHRLWAAHCRKIQLKKDGSSNHVYNYQPCDHPRQPCDSSCPCVIAQNFCEKFCQCSSECQNRFPGCRCKAQCNTKQCPCYLAVRECDPDLCLTCGAADHWDSKNVSCKNCSIQRGSKKHLLLAPSDVAGWGIFIKDPVQKNEFISEYCGEIISQDEADRRGKVYDKYMCSFLFNLNNDFVVDATRKGNKIRFANHSVNPNCYAKVMMVNGDHRIGIFAKRAIQTGEELFFDYRYSQADALKYVGIEREMEIP

SEQ ID NO: 2 - DNA, 2723 bp, Homo sapiens
ggcggcgcttgattgggctgggggggccaaataaaagcgatggcgattgggctgccgcgtttggcgctcggtccggtcgcgtccgacacccggtgggactcagaaggcagtggagccccggcggcggcggcggcggcgcgcgggggcgacgcgcgggaacaacgcgagtcggcgcgcgggacgaagaataatcatgggccagactgggaagaaatctgagaagggaccagtttgttggcggaagcgtgtaaaatcagagtacatgcgactgagacagctcaagaggttcagacgagctgatgaagtaaagagtatgtttagttccaatcgtcagaaaattttggaaagaacggaaatcttaaaccaagaatggaaacagcgaaggatacagcctgtgcacatcctgacttctgtgagctcattgcgcgggactagggagtgttcggtgaccagtgacttggattttccaacacaagtcatcccattaaagactctgaatgcagttgcttcagtacccataatgtattcttggtctcccctacagcagaattttatggtggaagatgaaactgttttacataacattccttatatgggagatgaagttttagatcaggatggtactttcattgaagaactaataaaaaattatgatgggaaagtacacggggatagagaatgtgggtttataaatgatgaaatttttgtggagttggtgaatgcccttggtcaatataatgatgatgacgatgatgatgatggagacgatcctgaagaaagagaagaaaagcagaaagatctggaggatcaccgagatgataaagaaagccgcccacctcggaaatttccttctgataaaatttttgaagccatttcctcaatgtttccagataagggcacagcagaagaactaaaggaaaaatataaagaactcaccgaacagcagctcccaggcgcacttcctcctgaatgtacccccaacatagatggaccaaatgctaaatctgttcagagagagcaaagcttacactcctttcatacgcttttctgtaggcgatgttttaaatatgactgcttcctacatcgtaagtgcaattattcttttcatgcaacacccaacacttataagcggaagaacacagaaacagctctagacaacaaaccttgtggaccacagtgttaccagcatttggagggagcaaaggagtttgctgctgctctcaccgctgagcggataaagaccccaccaaaacgtccaggaggccgcagaagaggacggcttcccaataacagtagcaggcccagcacccccaccattaatgtgctggaatcaaaggatacagacagtgatagggaagcagggactgaaacggggggagagaacaatgataaagaagaagaagagaagaaagatgaaacttcgagctcctctgaagcaaattctcggtgtcaaacaccaataaagatgaagccaaatattgaacctcctgagaatgtggagtggagtggtgctgaagcctcaatgtttagagtcctcattggcacttactatgacaatttctgtgccattgctaggttaattgggaccaaaacatgtagacaggtgtatgagtttagagtcaaagaatctagcatcatagctccagctcccgctgaggatgtggatactcctccaaggaaaaagaagaggaaacaccggttgtgggctgcacactgcagaaagatacagctgaaaaaggacggctcctctaaccatgtttacaactatcaaccctgtgatcatccacggcagccttgtgacagttcgtgcccttgtgtgatagcacaaaatttttgtgaaaagttttgtcaatgtagttcagagtgtcaaaaccgctttccgggatgccgctgcaaagcacagtgcaacaccaagcagtgcccgtgctacctggctgtccgagagtgtgaccctgacctctgtcttacttgtggagccgctgaccattgggacagtaaaaatgtgtcctgcaagaactgcagtattcagcggggctccaaaaagcatctattgctggcaccatctgacgtggcaggctgggggatttttatcaaagatcctgtgcagaaaaatgaattcatctcagaatactgtggagagattatttctcaagatgaagctgacagaagagggaaagtgtatgataaatacatgtgcagctttctgttcaacttgaacaatgattttgtggtggatgcaacccgcaagggtaacaaaattcgttttgcaaatcattcggtaaatccaaactgctatgcaaaagttatgatggttaacggtgatcacaggataggtatttttgccaagagagccatccagactggcgaagagctgttttttgattacagatacagccaggctgatgccctgaagtatgtcggcatcgaaagagaaatggaaatcccttgacatctgctacctcctcccccctcctctgaaacagctgccttagcttcaggaacctcgagtactgtgggcaatttagaaaaagaacatgcagtttgaaattctgaatttgcaaagtactgtaagaataatttatagtaatgagtttaaaaatcaactttttattgccttctcaccagctgcaaagtgttttgtaccagtgaatttttgcaataatgcagtatggtacatttttcaactttgaataaagaatacttgaacttgtccttgttgaatc

SEQ ID NO: 3 - protein, 751 aa, Homo sapiens
MGQTGKKSEKGPVCWRKRVKSEYMRLRQLKRFRRADEVKSMFSSNRQKILERTEILNQEWKQRRIQPVHILTSVSSLRGTRECSVTSDLDFPTQVIPLKTLNAVASVPIMYSWSPLQQNFMVEDETVLHNIPYMGDEVLDQDGTFIEELIKNYDGKVHGDRECGFINDEIFVELVNALGQYNDDDDDDDGDDPEEREEKQKDLEDHRDDKESRPPRKFPSDKIFEAISSMFPDKGTAEELKEKYKELTEQQLPGALPPECTPNIDGPNAKSVQREQSLHSFHTLFCRRCFKYDCFLHRKCNYSFHATPNTYKRKNTETALDNKPCGPQCYQHLEGAKEFAAALTAERIKTPPKRPGGRRRGRLPNNSSRPSTPTINVLESKDTDSDREAGTETGGENNDKEEEEKKDETSSSSEANSRCQTPIKMKPNIEPPENVEWSGAEASMFRVLIGTYYDNFCAIARLIGTKTCRQVYEFRVKESSIIAPAPAEDVDTPPRKKKRKHRLWAAHCRKIQLKKDGSSNHVYNYQPCDHPRQPCDSSCPCVIAQNFCEKFCQCSSECQNRFPGCRCKAQCNTKQCPCYLAVRECDPDLCLTCGAADHWDSKNVSCKNCSIQRGSKKHLLLAPSDVAGWGIFIKDPVQKNEFISEYCGEIISQDEADRRGKVYDKYMCSFLFNLNNDFVVDATRKGNKIRFANHSVNPNCYAKVMMVNGDHRIGIFAKRAIQTGEELFFDYRYSQADALKYVGIEREMEIP

SEQ ID NO: 4 - DNA, 2591 bp, Homo sapiens
ggcggcgcttgattgggctgggggggccaaataaaagcgatggcgattgggctgccgcgtttggcgctcggtccggtcgcgtccgacacccggtgggactcagaaggcagtggagccccggcggcggcggcggcggcgcgcgggggcgacgcgcgggaacaacgcgagtcggcgcgcgggacgaagaataatcatgggccagactgggaagaaatctgagaagggaccagtttgttggcggaagcgtgtaaaatcagagtacatgcgactgagacagctcaagaggttcagacgagctgatgaagtaaagagtatgtttagttccaatcgtcagaaaattttggaaagaacggaaatcttaaaccaagaatggaaacagcgaaggatacagcctgtgcacatcctgacttctgtgagctcattgcgcgggactagggaggtggaagatgaaactgttttacataacattccttatatgggagatgaagttttagatcaggatggtactttcattgaagaactaataaaaaattatgatgggaaagtacacggggatagagaatgtgggtttataaatgatgaaatttttgtggagttggtgaatgcccttggtcaatataatgatgatgacgatgatgatgatggagacgatcctgaagaaagagaagaaaagcagaaagatctggaggatcaccgagatgataaagaaagccgcccacctcggaaatttccttctgataaaatttttgaagccatttcctcaatgtttccagataagggcacagcagaagaactaaaggaaaaatataaagaactcaccgaacagcagctcccaggcgcacttcctcctgaatgtacccccaacatagatggaccaaatgctaaatctgttcagagagagcaaagcttacactcctttcatacgcttttctgtaggcgatgttttaaatatgactgcttcctacatccttttcatgcaacacccaacacttataagcggaagaacacagaaacagctctagacaacaaaccttgtggaccacagtgttaccagcatttggagggagcaaaggagtttgctgctgctctcaccgctgagcggataaagaccccaccaaaacgtccaggaggccgcagaagaggacggcttcccaataacagtagcaggcccagcacccccaccattaatgtgctggaatcaaaggatacagacagtgatagggaagcagggactgaaacggggggagagaacaatgataaagaagaagaagagaagaaagatgaaacttcgagctcctctgaagcaaattctcggtgtcaaacaccaataaagatgaagccaaatattgaacctcctgagaatgtggagtggagtggtgctgaagcctcaatgtttagagtcctcattggcacttactatgacaatttctgtgccattgctaggttaattgggaccaaaacatgtagacaggtgtatgagtttagagtcaaagaatctagcatcatagctccagctcccgctgaggatgtggatactcctccaaggaaaaagaagaggaaacaccggttgtgggctgcacactgcagaaagatacagctgaaaaaggacggctcctctaaccatgtttacaactatcaaccctgtgatcatccacggcagccttgtgacagttcgtgcccttgtgtgatagcacaaaatttttgtgaaaagttttgtcaatgtagttcagagtgtcaaaaccgctttccgggatgccgctgcaaagcacagtgcaacaccaagcagtgcccgtgctacctggctgtccgagagtgtgaccctgacctctgtcttacttgtggagccgctgaccattgggacagtaaaaatgtgtcctgcaagaactgcagtattcagcggggctccaaaaagcatctattgctggcaccatctgacgtggcaggctgggggatttttatcaaagatcctgtgcagaaaaatgaattcatctcagaatactgtggagagattatttctcaagatgaagctgacagaagagggaaagtgtatgataaatacatgtgcagctttctgttcaacttgaacaatgattttgtggtggatgcaacccgcaagggtaacaaaattcgttttgcaaatcattcggtaaatccaaactgctatgcaaaagttatgatggttaacggtgatcacaggataggtatttttgccaagagagccatccagactggcgaagagctgttttttgattacagatacagccaggctgatgccctgaagtatgtcggcatcgaaagagaaatggaaatcccttgacatctgctacctcctcccccctcctctgaaacagctgccttagcttcaggaacctcgagtactgtgggcaatttagaaaaagaacatgcagtttgaaattctgaatttgcaaagtactgtaagaataatttatagtaatgagtttaaaaatcaactttttattgccttctcaccagctgcaaagtgttttgtaccagtgaatttttgcaataatgcagtatggtacatttttcaactttgaataaagaatacttgaacttgtccttgttgaatc

SEQ ID NO: 5 - protein, 707 aa, Homo sapiens
MGQTGKKSEKGPVCWRKRVKSEYMRLRQLKRFRRADEVKSMFSSNRQKILERTEILNQEWKQRRIQPVHILTSVSSLRGTREVEDETVLHNIPYMGDEVLDQDGTFIEELIKNYDGKVHGDRECGFINDEIFVELVNALGQYNDDDDDDDGDDPEEREEKQKDLEDHRDDKESRPPRKFPSDKIFEAISSMFPDKGTAEELKEKYKELTEQQLPGALPPECTPNIDGPNAKSVQREQSLHSFHTLFCRRCFKYDCFLHPFHATPNTYKRKNTETALDNKPCGPQCYQHLEGAKEFAAALTAERIKTPPKRPGGRRRGRLPNNSSRPSTPTINVLESKDTDSDREAGTETGGENNDKEEEEKKDETSSSSEANSRCQTPIKMKPNIEPPENVEWSGAEASMFRVLIGTYYDNFCAIARLIGTKTCRQVYEFRVKESSIIAPAPAEDVDTPPRKKKRKHRLWAAHCRKIQLKKDGSSNHVYNYQPCDHPRQPCDSSCPCVIAQNFCEKFCQCSSECQNRFPGCRCKAQCNTKQCPCYLAVRECDPDLCLTCGAADHWDSKNVSCKNCSIQRGSKKHLLLAPSDVAGWGIFIKDPVQKNEFISEYCGEIISQDEADRRGKVYDKYMCSFLFNLNNDFVVDATRKGNKIRFANHSVNPNCYAKVMMVNGDHRIGIFAKRAIQTGEELFFDYRYSQADALKYVGIEREMEIP

SEQ ID NO: 6 - protein, 200 aa, Homo sapiens
SCPCVIAQNFCEKFCQCSSECQNRFPGCRCKAQCNTKQCPCYLAVRECDPDLCLTCGAADHWDSKNVSCKNCSIQRGSKKHLLLAPSDVAGWGIFIKDPVQKNEFISEYCGEIISQDEADRRGKVYDKYMCSFLFNLNNDFVVDATRKGNKIRFANHSVNPNCYAKVMMVNGDHRIGIFAKRAIQTGEELFFDYRYSQAD

SEQ ID NO: 7 - protein, 114 aa, Homo sapiens
HLLLAPSDVAGWGIFIKDPVQKNEFISEYCGEIISQDEADRRGKVYDKYMCSFLFNLNNDFVVDATRKGNKIRFANHSVNPNCYAKVMMVNGDHRIGIFAKRAIQTGEELFFDY

SEQ ID NO: 8 - DNA, 342 bp, Homo sapiens
catctattgctggcaccatctgacgtggcaggctgggggatttttatcaaagatcctgtgcagaaaaatgaattcatctcagaatactgtggagagattatttctcaagatgaagctgacagaagagggaaagtgtatgataaatacatgtgcagctttctgttcaacttgaacaatgattttgtggtggatgcaacccgcaagggtaacaaaattcgttttgcaaatcattcggtaaatccaaactgctatgcaaaagttatgatggttaacggtgatcacaggataggtatttttgccaagagagccatccagactggcgaagagctgttttttgattac

SEQ ID NO: 9 - protein, 746 aa, Homo sapiens
  MISC_FEATURE (641)..(641): Wherein "X" can be any amino acid residue other than tyrosine "Y"
MGQTGKKSEKGPVCWRKRVKSEYMRLRQLKRFRRADEVKSMFSSNRQKILERTEILNQEWKQRRIQPVHILTSVSSLRGTRECSVTSDLDFPTQVIPLKTLNAVASVPIMYSWSPLQQNFMVEDETVLHNIPYMGDEVLDQDGTFIEELIKNYDGKVHGDRECGFINDEIFVELVNALGQYNDDDDDDDGDDPEEREEKQKDLEDHRDDKESRPPRKFPSDKIFEAISSMFPDKGTAEELKEKYKELTEQQLPGALPPECTPNIDGPNAKSVQREQSLHSFHTLFCRRCFKYDCFLHPFHATPNTYKRKNTETALDNKPCGPQCYQHLEGAKEFAAALTAERIKTPPKRPGGRRRGRLPNNSSRPSTPTINVLESKDTDSDREAGTETGGENNDKEEEEKKDETSSSSEANSRCQTPIKMKPNIEPPENVEWSGAEASMFRVLIGTYYDNFCAIARLIGTKTCRQVYEFRVKESSIIAPAPAEDVDTPPRKKKRKHRLWAAHCRKIQLKKDGSSNHVYNYQPCDHPRQPCDSSCPCVIAQNFCEKFCQCSSECQNRFPGCRCKAQCNTKQCPCYLAVRECDPDLCLTCGAADHWDSKNVSCKNCSIQRGSKKHLLLAPSDVAGWGIFIKDPVQKNEFISEXCGEIISQDEADRRGKVYDKYMCSFLFNLNNDFVVDATRKGNKIRFANHSVNPNCYAKVMMVNGDHRIGIFAKRAIQTGEELFFDYRYSQADALKYVGIEREMEIP

SEQ ID NO: 10 - protein, 746 aa, Homo sapiens
MGQTGKKSEKGPVCWRKRVKSEYMRLRQLKRFRRADEVKSMFSSNRQKILERTEILNQEWKQRRIQPVHILTSVSSLRGTRECSVTSDLDFPTQVIPLKTLNAVASVPIMYSWSPLQQNFMVEDETVLHNIPYMGDEVLDQDGTFIEELIKNYDGKVHGDRECGFINDEIFVELVNALGQYNDDDDDDDGDDPEEREEKQKDLEDHRDDKESRPPRKFPSDKIFEAISSMFPDKGTAEELKEKYKELTEQQLPGALPPECTPNIDGPNAKSVQREQSLHSFHTLFCRRCFKYDCFLHPFHATPNTYKRKNTETALDNKPCGPQCYQHLEGAKEFAAALTAERIKTPPKRPGGRRRGRLPNNSSRPSTPTINVLESKDTDSDREAGTETGGENNDKEEEEKKDETSSSSEANSRCQTPIKMKPNIEPPENVEWSGAEASMFRVLIGTYYDNFCAIARLIGTKTCRQVYEFRVKESSIIAPAPAEDVDTPPRKKKRKHRLWAAHCRKIQLKKDGSSNHVYNYQPCDHPRQPCDSSCPCVIAQNFCEKFCQCSSECQNRFPGCRCKAQCNTKQCPCYLAVRECDPDLCLTCGAADHWDSKNVSCKNCSIQRGSKKHLLLAPSDVAGWGIFIKDPVQKNEFISEFCGEIISQDEADRRGKVYDKYMCSFLFNLNNDFVVDATRKGNKIRFANHSVNPNCYAKVMMVNGDHRIGIFAKRAIQTGEELFFDYRYSQADALKYVGIEREMEIP

SEQ ID NO: 11 - protein, 746 aa, Homo sapiens
MGQTGKKSEKGPVCWRKRVKSEYMRLRQLKRFRRADEVKSMFSSNRQKILERTEILNQEWKQRRIQPVHILTSVSSLRGTRECSVTSDLDFPTQVIPLKTLNAVASVPIMYSWSPLQQNFMVEDETVLHNIPYMGDEVLDQDGTFIEELIKNYDGKVHGDRECGFINDEIFVELVNALGQYNDDDDDDDGDDPEEREEKQKDLEDHRDDKESRPPRKFPSDKIFEAISSMFPDKGTAEELKEKYKELTEQQLPGALPPECTPNIDGPNAKSVQREQSLHSFHTLFCRRCFKYDCFLHPFHATPNTYKRKNTETALDNKPCGPQCYQHLEGAKEFAAALTAERIKTPPKRPGGRRRGRLPNNSSRPSTPTINVLESKDTDSDREAGTETGGENNDKEEEEKKDETSSSSEANSRCQTPIKMKPNIEPPENVEWSGAEASMFRVLIGTYYDNFCAIARLIGTKTCRQVYEFRVKESSIIAPAPAEDVDTPPRKKKRKHRLWAAHCRKIQLKKDGSSNHVYNYQPCDHPRQPCDSSCPCVIAQNFCEKFCQCSSECQNRFPGCRCKAQCNTKQCPCYLAVRECDPDLCLTCGAADHWDSKNVSCKNCSIQRGSKKHLLLAPSDVAGWGIFIKDPVQKNEFISEHCGEIISQDEADRRGKVYDKYMCSFLFNLNNDFVVDATRKGNKIRFANHSVNPNCYAKVMMVNGDHRIGIFAKRAIQTGEELFFDYRYSQADALKYVGIEREMEIP

SEQ ID NO: 12 - protein, 746 aa, Homo sapiens
MGQTGKKSEKGPVCWRKRVKSEYMRLRQLKRFRRADEVKSMFSSNRQKILERTEILNQEWKQRRIQPVHILTSVSSLRGTRECSVTSDLDFPTQVIPLKTLNAVASVPIMYSWSPLQQNFMVEDETVLHNIPYMGDEVLDQDGTFIEELIKNYDGKVHGDRECGFINDEIFVELVNALGQYNDDDDDDDGDDPEEREEKQKDLEDHRDDKESRPPRKFPSDKIFEAISSMFPDKGTAEELKEKYKELTEQQLPGALPPECTPNIDGPNAKSVQREQSLHSFHTLFCRRCFKYDCFLHPFHATPNTYKRKNTETALDNKPCGPQCYQHLEGAKEFAAALTAERIKTPPKRPGGRRRGRLPNNSSRPSTPTINVLESKDTDSDREAGTETGGENNDKEEEEKKDETSSSSEANSRCQTPIKMKPNIEPPENVEWSGAEASMFRVLIGTYYDNFCAIARLIGTKTCRQVYEFRVKESSIIAPAPAEDVDTPPRKKKRKHRLWAAHCRKIQLKKDGSSNHVYNYQPCDHPRQPCDSSCPCVIAQNFCEKFCQCSSECQNRFPGCRCKAQCNTKQCPCYLAVRECDPDLCLTCGAADHWDSKNVSCKNCSIQRGSKKHLLLAPSDVAGWGIFIKDPVQKNEFISENCGEIISQDEADRRGKVYDKYMCSFLFNLNNDFVVDATRKGNKIRFANHSVNPNCYAKVMMVNGDHRIGIFAKRAIQTGEELFFDYRYSQADALKYVGIEREMEIP

SEQ ID NO: 13 - protein, 746 aa, Homo sapiens
MGQTGKKSEKGPVCWRKRVKSEYMRLRQLKRFRRADEVKSMFSSNRQKILERTEILNQEWKQRRIQPVHILTSVSSLRGTRECSVTSDLDFPTQVIPLKTLNAVASVPIMYSWSPLQQNFMVEDETVLHNIPYMGDEVLDQDGTFIEELIKNYDGKVHGDRECGFINDEIFVELVNALGQYNDDDDDDDGDDPEEREEKQKDLEDHRDDKESRPPRKFPSDKIFEAISSMFPDKGTAEELKEKYKELTEQQLPGALPPECTPNIDGPNAKSVQREQSLHSFHTLFCRRCFKYDCFLHPFHATPNTYKRKNTETALDNKPCGPQCYQHLEGAKEFAAALTAERIKTPPKRPGGRRRGRLPNNSSRPSTPTINVLESKDTDSDREAGTETGGENNDKEEEEKKDETSSSSEANSRCQTPIKMKPNIEPPENVEWSGAEASMFRVLIGTYYDNFCAIARLIGTKTCRQVYEFRVKESSIIAPAPAEDVDTPPRKKKRKHRLWAAHCRKIQLKKDGSSNHVYNYQPCDHPRQPCDSSCPCVIAQNFCEKFCQCSSECQNRFPGCRCKAQCNTKQCPCYLAVRECDPDLCLTCGAADHWDSKNVSCKNCSIQRGSKKHLLLAPSDVAGWGIFIKDPVQKNEFISESCGEIISQDEADRRGKVYDKYMCSFLFNLNNDFVVDATRKGNKIRFANHSVNPNCYAKVMMVNGDHRIGIFAKRAIQTGEELFFDYRYSQADALKYVGIEREMEIP

SEQ ID NO: 14 - protein, 746 aa, Homo sapiens
MGQTGKKSEKGPVCWRKRVKSEYMRLRQLKRFRRADEVKSMFSSNRQKILERTEILNQEWKQRRIQPVHILTSVSSLRGTRECSVTSDLDFPTQVIPLKTLNAVASVPIMYSWSPLQQNFMVEDETVLHNIPYMGDEVLDQDGTFIEELIKNYDGKVHGDRECGFINDEIFVELVNALGQYNDDDDDDDGDDPEEREEKQKDLEDHRDDKESRPPRKFPSDKIFEAISSMFPDKGTAEELKEKYKELTEQQLPGALPPECTPNIDGPNAKSVQREQSLHSFHTLFCRRCFKYDCFLHPFHATPNTYKRKNTETALDNKPCGPQCYQHLEGAKEFAAALTAERIKTPPKRPGGRRRGRLPNNSSRPSTPTINVLESKDTDSDREAGTETGGENNDKEEEEKKDETSSSSEANSRCQTPIKMKPNIEPPENVEWSGAEASMFRVLIGTYYDNFCAIARLIGTKTCRQVYEFRVKESSIIAPAPAEDVDTPPRKKKRKHRLWAAHCRKIQLKKDGSSNHVYNYQPCDHPRQPCDSSCPCVIAQNFCEKFCQCSSECQNRFPGCRCKAQCNTKQCPCYLAVRECDPDLCLTCGAADHWDSKNVSCKNCSIQRGSKKHLLLAPSDVAGWGIFIKDPVQKNEFISECCGEIISQDEADRRGKVYDKYMCSFLFNLNNDFVVDATRKGNKIRFANHSVNPNCYAKVMMVNGDHRIGIFAKRAIQTGEELFFDYRYSQADALKYVGIEREMEIP

SEQ ID NO: 15 - protein, 746 aa, Homo sapiens
  MISC_FEATURE (677)..(677): Wherein X is preferably a glycine "G"
MGQTGKKSEKGPVCWRKRVKSEYMRLRQLKRFRRADEVKSMFSSNRQKILERTEILNQEWKQRRIQPVHILTSVSSLRGTRECSVTSDLDFPTQVIPLKTLNAVASVPIMYSWSPLQQNFMVEDETVLHNIPYMGDEVLDQDGTFIEELIKNYDGKVHGDRECGFINDEIFVELVNALGQYNDDDDDDDGDDPEEREEKQKDLEDHRDDKESRPPRKFPSDKIFEAISSMFPDKGTAEELKEKYKELTEQQLPGALPPECTPNIDGPNAKSVQREQSLHSFHTLFCRRCFKYDCFLHPFHATPNTYKRKNTETALDNKPCGPQCYQHLEGAKEFAAALTAERIKTPPKRPGGRRRGRLPNNSSRPSTPTINVLESKDTDSDREAGTETGGENNDKEEEEKKDETSSSSEANSRCQTPIKMKPNIEPPENVEWSGAEASMFRVLIGTYYDNFCAIARLIGTKTCRQVYEFRVKESSIIAPAPAEDVDTPPRKKKRKHRLWAAHCRKIQLKKDGSSNHVYNYQPCDHPRQPCDSSCPCVIAQNFCEKFCQCSSECQNRFPGCRCKAQCNTKQCPCYLAVRECDPDLCLTCGAADHWDSKNVSCKNCSIQRGSKKHLLLAPSDVAGWGIFIKDPVQKNEFISEYCGEIISQDEADRRGKVYDKYMCSFLFNLNNDFVVDXTRKGNKIRFANHSVNPNCYAKVMMVNGDHRIGIFAKRAIQTGEELFFDYRYSQADALKYVGIEREMEIP

SEQ ID NO: 16 - protein, 746 aa, Homo sapiens
  MISC_FEATURE (687)..(687): Wherein X is preferably a valine "V"
MGQTGKKSEKGPVCWRKRVKSEYMRLRQLKRFRRADEVKSMFSSNRQKILERTEILNQEWKQRRIQPVHILTSVSSLRGTRECSVTSDLDFPTQVIPLKTLNAVASVPIMYSWSPLQQNFMVEDETVLHNIPYMGDEVLDQDGTFIEELIKNYDGKVHGDRECGFINDEIFVELVNALGQYNDDDDDDDGDDPEEREEKQKDLEDHRDDKESRPPRKFPSDKIFEAISSMFPDKGTAEELKEKYKELTEQQLPGALPPECTPNIDGPNAKSVQREQSLHSFHTLFCRRCFKYDCFLHPFHATPNTYKRKNTETALDNKPCGPQCYQHLEGAKEFAAALTAERIKTPPKRPGGRRRGRLPNNSSRPSTPTINVLESKDTDSDREAGTETGGENNDKEEEEKKDETSSSSEANSRCQTPIKMKPNIEPPENVEWSGAEASMFRVLIGTYYDNFCAIARLIGTKTCRQVYEFRVKESSIIAPAPAEDVDTPPRKKKRKHRLWAAHCRKIQLKKDGSSNHVYNYQPCDHPRQPCDSSCPCVIAQNFCEKFCQCSSECQNRFPGCRCKAQCNTKQCPCYLAVRECDPDLCLTCGAADHWDSKNVSCKNCSIQRGSKKHLLLAPSDVAGWGIFIKDPVQKNEFISEYCGEIISQDEADRRGKVYDKYMCSFLFNLNNDFVVDATRKGNKIRFXNHSVNPNCYAKVMMVNGDHRIGIFAKRAIQTGEELFFDYRYSQADALKYVGIEREMEIP

SEQ ID NO: 17 - protein, 746 aa, Homo sapiens
  MISC_FEATURE (685)..(685): Wherein X is preferably a cysteine "C" or a histidine "H"
MGQTGKKSEKGPVCWRKRVKSEYMRLRQLKRFRRADEVKSMFSSNRQKILERTEILNQEWKQRRIQPVHILTSVSSLRGTRECSVTSDLDFPTQVIPLKTLNAVASVPIMYSWSPLQQNFMVEDETVLHNIPYMGDEVLDQDGTFIEELIKNYDGKVHGDRECGFINDEIFVELVNALGQYNDDDDDDDGDDPEEREEKQKDLEDHRDDKESRPPRKFPSDKIFEAISSMFPDKGTAEELKEKYKELTEQQLPGALPPECTPNIDGPNAKSVQREQSLHSFHTLFCRRCFKYDCFLHPFHATPNTYKRKNTETALDNKPCGPQCYQHLEGAKEFAAALTAERIKTPPKRPGGRRRGRLPNNSSRPSTPTINVLESKDTDSDREAGTETGGENNDKEEEEKKDETSSSSEANSRCQTPIKMKPNIEPPENVEWSGAEASMFRVLIGTYYDNFCAIARLIGTKTCRQVYEFRVKESSIIAPAPAEDVDTPPRKKKRKHRLWAAHCRKIQLKKDGSSNHVYNYQPCDHPRQPCDSSCPCVIAQNFCEKFCQCSSECQNRFPGCRCKAQCNTKQCPCYLAVRECDPDLCLTCGAADHWDSKNVSCKNCSIQRGSKKHLLLAPSDVAGWGIFIKDPVQKNEFISEYCGEIISQDEADRRGKVYDKYMCSFLFNLNNDFVVDATRKGNKIXFANHSVNPNCYAKVMMVNGDHRIGIFAKRAIQTGEELFFDYRYSQADALKYVGIEREMEIP

SEQ ID NO: 18 - protein, 745 aa, Homo sapiens
  MISC_FEATURE (641)..(729): Wherein "X" can be any amino acid except the corresponding wild type residue.
MGQTGKKSEKGPVCWRKRVKSEYMRLRQLKRFRRADEVKSMFSSNRQKILERTEILNQEWKQRRIQPVHILTSVSSLRGTRECSVTSDLDFPTQVIPLKTLNAVASVPIMYSWSPLQQNFMVEDETVLHNIPYMGDEVLDQDGTFIEELIKNYDGKVHGDRECGFINDEIFVELVNALGQYNDDDDDDDGDDPEEREEKQKDLEDHRDDKESRPPRKFPSDKIFEAISSMFPDKGTAEELKEKYKELTEQQLPGALPPECTPNIDGPNAKSVQREQSLHSFHTLFCRRCFKYDCFLHPFHATPNTYKRKNTETALDNKPCGPQCYQHLEGAKEFAAALTAERIKTPPKRPGGRRRGRLPNNSSRPSTPTINVLESKDTDSDREAGTETGGENNDKEEEEKKDETSSSSEANSRCQTPIKMKPNIEPPENVEWSGAEASMFRVLIGTYYDNFCAIARLIGTKTCRQVYEFRVKESSIIAPAPAEDVDTPPRKKKRKHRLWAAHCRKIQLKKDGSSNHVYNYQPCDHPRQPCDSSCPCVIAQNFCEKFCQCSSECQNRFPGCRCKAQCNTKQCPCYLAVRECDPDLCLTCGAADHWDSKNVSCKNCSIQRGSKKHLLLAPSDVAGWGIFIKDPVQKNEFISEXCGEIISQDEADRRGKVYDKYMXXXLXNLNNDFXXDXTRKGNKXXXXHSVNPNCYAKVMMVNGDHRXGIFAKRAIQTGEELFXDXRYSXADALKYVGIEREMEIP

SEQ ID NO: 19 - DNA, 8761 bp, Homo sapiens
gccgaggaggaagaggttgatggcggcggcggagctccgagagacctcggctgggcaggggccggccgtggcgggccggggactgcgcctctagagccgcgagttctcgggaattcgccgcagcggacgcgctcggcgaatttgtgctcttgtgccctcctccgggcttgggcccaggcccggcccctcgcacttgcccttaccttttctatcgagtccgcatccctctccagccactgcgacccggcgaagagaaaaaggaacttcccccaccccctcgggtgccgtcggagccccccagcccacccctgggtgcggcgcggggaccccgggccgaagaagagatttcctgaggattctggttttcctcgcttgtatctccgaaagaattaaaaatggccgagaatgtggtggaaccggggccgccttcagccaagcggcctaaactctcatctccggccctctcggcgtccgccagcgatggcacagattttggctctctatttgacttggagcacgacttaccagatgaattaatcaactctacagaattgggactaaccaatggtggtgatattaatcagcttcagacaagtcttggcatggtacaagatgcagcttctaaacataaacagctgtcagaattgctgcgatctggtagttcccctaacctcaatatgggagttggtggcccaggtcaagtcatggccagccaggcccaacagagcagtcctggattaggtttgataaatagcatggtcaaaagcccaatgacacaggcaggcttgacttctcccaacatggggatgggcactagtggaccaaatcagggtcctacgcagtcaacaggtatgatgaacagtccagtaaatcagcctgccatgggaatgaacacagggatgaatgcgggcatgaatcctggaatgttggctgcaggcaatggacaagggataatgcctaatcaagtcatgaacggttcaattggagcaggccgagggcgacagaatatgcagtacccaaacccaggcatgggaagtgctggcaacttactgactgagcctcttcagcagggctctccccagatgggaggacaaacaggattgagaggcccccagcctcttaagatgggaatgatgaacaaccccaatccttatggttcaccatatactcagaatcctggacagcagattggagccagtggccttggtctccagattcagacaaaaactgtactatcaaataacttatctccatttgctatggacaaaaaggcagttcctggtggaggaatgcccaacatgggtcaacagccagccccgcaggtccagcagccaggcctggtgactccagttgcccaagggatgggttctggagcacatacagctgatccagagaagcgcaagctcatccagcagcagcttgttctccttttgcatgctcacaagtgccagcgccgggaacaggccaatggggaagtgaggcagtgcaaccttccccactgtcgcacaatgaagaatgtcctaaaccacatgacacactgccagtcaggcaagtcttgccaagtggcacactgtgcatcttctcgacaaatcatttcacactggaagaattgtacaagacatgattgtcctgtgtgtctccccctcaaaaatgctggtgataagagaaatcaacagccaattttgactggagcacccgttggacttggaaatcctagctctctaggggtgggtcaacagtctgcccccaacctaagcactgttagtcagattgatcccagctccatagaaagagcctatgcagctcttggactaccctatcaagtaaatcagatgccgacacaaccccaggtgcaagcaaagaaccagcagaatcagcagcctgggcagtctccccaaggcatgcggcccatgagcaacatgagtgctagtcctatgggagtaaatggaggtgtaggagttcaaacgccgagtcttctttctgactcaatgttgcattcagccataaattctcaaaacccaatgatgagtgaaaatgccagtgtgccctccctgggtcctatgccaacagcagctcaaccatccactactggaattcggaaacagtggcacgaagatattactcaggatcttcgaaatcatcttgttcacaaactcgtccaagccatatttcctacgccggatcctgctgctttaaaagacagacggatggaaaacctagttgcatatgctcggaaagttgaaggggacatgtatgaatctgcaaacaatcgagcggaatactaccaccttctagctgagaaaatctataagatccagaaagaactagaagaaaaacgaaggaccagactacagaagcagaacatgctaccaaatgctgcaggcatggttccagtttccatgaatccagggcctaacatgggacagccgcaaccaggaatgacttctaatggccctctacctgacccaagtatgatccgtggcagtgtgccaaaccagatgatgcctcgaataactccacaatctggtttgaatcaatttggccagatgagcatggcccagccccctattgtaccccggcaaacccctcctcttcagcaccatggacagttggctcaacctggagctctcaacccgcctatgggctatgggcctcgtatgcaacagccttccaaccagggccagttccttcctcagactcagttcccatcacagggaatgaatgtaacaaatatccctttggctccgtccagcggtcaagctccagtgtctcaagcacaaatgtctagttcttcctgcccggtgaactctcctataatgcctccagggtctcaggggagccacattcactgtccccagcttcctcaaccagctcttcatcagaattcaccctcgcctgtacctagtcgtacccccacccctcaccatactcccccaagcataggggctcagcagccaccagcaacaacaattccagcccctgttcctacacctcctgccatgccacctgggccacagtcccaggctctacatccccctccaaggcagacacctacaccaccaacaacacaacttccccaacaagtgcagccttcacttcctgctgcaccttctgctgaccagccccagcagcagcctcgctcacagcagagcacagcagcgtctgttcctaccccaacagcaccgctgcttcctccgcagcctgcaactccactttcccagccagctgtaagcattgaaggacaggtatcaaatcctccatctactagtagcacagaagtgaattctcaggccattgctgagaagcagccttcccaggaagtgaagatggaggccaaaatggaagtggatcaaccagaaccagcagatactcagccggaggatatttcagagtctaaagtggaagactgtaaaatggaatctaccgaaacagaagagagaagcactgagttaaaaactgaaataaaagaggaggaagaccagccaagtacttcagctacccagtcatctccggctccaggacagtcaaagaaaaagattttcaaaccagaagaactacgacaggcactgatgccaactttggaggcactttaccgtcaggatccagaatcccttccctttcgtcaacctgtggaccctcagcttttaggaatccctgattactttgatattgtgaagagccccatggatctttctaccattaagaggaagttagacactggacagtatcaggagccctggcagtatgtcgatgatatttggcttatgttcaataatgcctggttatataaccggaaaacatcacgggtatacaaatactgctccaagctctctgaggtctttgaacaagaaattgacccagtgatgcaaagccttggatactgttgtggcagaaagttggagttctctccacagacactgtgttgctacggcaaacagttgtgcacaatacctcgtgatgccacttattacagttaccagaacaggtatcatttctgtgagaagtgtttcaatgagatccaaggggagagcgtttctttgggggatgacccttcccagcctcaaactacaataaataaagaacaattttccaagagaaaaaatgacacactggatcctgaactgtttgttgaatgtacagagtgcggaagaaagatgcatcagatctgtgtccttcaccatgagatcatctggcctgctggattcgtctgtgatggctgtttaaagaaaagtgcacgaactaggaaagaaaataagttttctgctaaaaggttgccatctaccagacttggcacctttctagagaatcgtgtgaatgactttctgaggcgacagaatcaccctgagtcaggagaggtcactgttagagtagttcatgcttctgacaaaaccgtggaagtaaaaccaggcatgaaagcaaggtttgtggacagtggagagatggcagaatcctttccataccgaaccaaagccctctttgcctttgaagaaattgatggtgttgacctgtgcttctttggcatgcatgttcaagagtatggctctgactgccctccacccaaccagaggagagtatacatatcttacctcgatagtgttcatttcttccgtcctaaatgcttgaggactgcagtctatcatgaaatcctaattggatatttagaatatgtcaagaaattaggttacacaacagggcatatttgggcatgtccaccaagtgagggagatgattatatcttccattgccatcctcctgaccagaagatacccaagcccaagcgactgcaggaatggtacaaaaaaatgcttgacaaggctgtatcagagcgtattgtccatgactacaaggatatttttaaacaagctactgaagatagattaacaagtgcaaaggaattgccttatttcgagggtgatttctggcccaatgttctggaagaaagcattaaggaactggaacaggaggaagaagagagaaaacgagaggaaaacaccagcaatgaaagcacagatgtgaccaagggagacagcaaaaatgctaaaaagaagaataataagaaaaccagcaaaaataagagcagcctgagtaggggcaacaagaagaaacccgggatgcccaatgtatctaacgacctctcacagaaactatatgccaccatggagaagcataaagaggtcttctttgtgatccgcctcattgctggccctgctgccaactccctgcctcccattgttgatcctgatcctctcatcccctgcgatctgatggatggtcgggatgcgtttctcacgctggcaagggacaagcacctggagttctcttcactccgaagagcccagtggtccaccatgtgcatgctggtggagctgcacacgcagagccaggaccgctttgtctacacctgcaatgaatgcaagcaccatgtggagacacgctggcactgtactgtctgtgaggattatgacttgtgtatcacctgctataacactaaaaaccatgaccacaaaatggagaaactaggccttggcttagatgatgagagcaacaaccagcaggctgcagccacccagagcccaggcgattctcgccgcctgagtatccagcgctgcatccagtctctggtccatgcttgccagtgtcggaatgccaattgctcactgccatcctgccagaagatgaagcgggttgtgcagcataccaagggttgcaaacggaaaaccaatggcgggtgccccatctgcaagcagctcattgccctctgctgctaccatgccaagcactgccaggagaacaaatgcccggtgccgttctgcctaaacatcaagcagaagctccggcagcaacagctgcagcaccgactacagcaggcccaaatgcttcgcaggaggatggccagcatgcagcggactggtgtggttgggcagcaacagggcctcccttcccccactcctgccactccaacgacaccaactggccaacagccaaccaccccgcagacgccccagcccacttctcagcctcagcctacccctcccaatagcatgccaccctacttgcccaggactcaagctgctggccctgtgtcccagggtaaggcagcaggccaggtgacccctccaacccctcctcagactgctcagccaccccttccagggcccccacctgcagcagtggaaatggcaatgcagattcagagagcagcggagacgcagcgccagatggcccacgtgcaaatttttcaaaggccaatccaacaccagatgcccccgatgactcccatggcccccatgggtatgaacccacctcccatgaccagaggtcccagtgggcatttggagccagggatgggaccgacagggatgcagcaacagccaccctggagccaaggaggattgcctcagccccagcaactacagtctgggatgccaaggccagccatgatgtcagtggcccagcatggtcaacctttgaacatggctccacaaccaggattgggccaggtaggtatcagcccactcaaaccaggcactgtgtctcaacaagccttacaaaaccttttgcggactctcaggtctcccagctctcccctgcagcagcaacaggtgcttagtatccttcacgccaacccccagctgttggctgcattcatcaagcagcgggctgccaagtatgccaactctaatccacaacccatccctgggcagcctggcatgccccaggggcagccagggctacagccacctaccatgccaggtcagcagggggtccactccaatccagccatgcagaacatgaatccaatgcaggcgggcgttcagagggctggcctgccccagcagcaaccacagcagcaactccagccacccatgggagggatgagcccccaggctcagcagatgaacatgaaccacaacaccatgccttcacaattccgagacatcttgagacgacagcaaatgatgcaacagcagcagcaacagggagcagggccaggaataggccctggaatggccaaccataaccagttccagcaaccccaaggagttggctacccaccacagcagcagcagcggatgcagcatcacatgcaacagatgcaacaaggaaatatgggacagataggccagcttccccaggccttgggagcagaggcaggtgccagtctacaggcctatcagcagcgactccttcagcaacagatggggtcccctgttcagcccaaccccatgagcccccagcagcatatgctcccaaatcaggcccagtccccacacctacaaggccagcagatccctaattctctctccaatcaagtgcgctctccccagcctgtcccttctccacggccacagtcccagcccccccactccagtccttccccaaggatgcagcctcagccttctccacaccacgtttccccacagacaagttccccacatcctggactggtagctgcccaggccaaccccatggaacaagggcattttgccagcccggaccagaattcaatgctttctcagcttgctagcaatccaggcatggcaaacctccatggtgcaagcgccacggacctgggactcagcaccgataactcagacttgaattcaaacctctcacagagtacactagacatacactagagacaccttgtagtattttgggagcaaaaaaattattttctcttaacaagactttttgtactgaaaacaatttttttgaatctttcgtagcctaaaagacaattttccttggaacacataagaactgtgcagtagccgtttgtggtttaaagcaaacatgcaagatgaacctgagggatgatagaatacaaagaatatatttttgttatggctggttaccaccagcctttcttcccctttgtgtgtgtggttcaagtgtgcactgggaggaggctgaggcctgtgaagccaaacaatatgctcctgccttgcacctccaataggttttattattttttttaaattaatgaacatatgtaatattaatagttattatttactggtgcagatggttgacatttttccctattttcctcactttatggaagagttaaaacatttctaaaccagaggacaaaaggggttaatgttactttaaaattacattctatatatatataaatatatataaatatatattaaaataccagttttttttctctgggtgcaaagatgttcattcttttaaaaaatgtttaaaaaaaaaaaaaaactgcctttcttcccctcaagtcaacttttgtgctccagaaaattttctattctgtaagtctgagcgtaaaacttcaagtattaaaataatttgtacatgtagagagaaaaatgactttttcaaaaatatacaggggcagctgccaaattgatgtattatatattgtggtttctgtttcttgaaagaatttttttcgttatttttacatctaacaaagtaaaaaaattaaaaagagggtaagaaacgattccggtgggatgattttaacatgcaaaatgtccctgggggtttcttctttgcttgctttcttcctccttaccctaccccccactcacacacacacacacacacacacacacacacacacacacacactttctataaaacttgaaaatagcaaaaaccctcaactgttgtaaatcatgcaattaaagttgattacttataaatatgaactttggatcactgtatagactgttaaatttgatttcttattacctattgttaaataaactgtgtgagacagaca

SEQ ID NO: 20 - protein, 2414 aa, Homo sapiens
MAENVVEPGPPSAKRPKLSSPALSASASDGTDFGSLFDLEHDLPDELINSTELGLTNGGDINQLQTSLGMVQDAASKHKQLSELLRSGSSPNLNMGVGGPGQVMASQAQQSSPGLGLINSMVKSPMTQAGLTSPNMGMGTSGPNQGPTQSTGMMNSPVNQPAMGMNTGMNAGMNPGMLAAGNGQGIMPNQVMNGSIGAGRGRQNMQYPNPGMGSAGNLLTEPLQQGSPQMGGQTGLRGPQPLKMGMMNNPNPYGSPYTQNPGQQIGASGLGLQIQTKTVLSNNLSPFAMDKKAVPGGGMPNMGQQPAPQVQQPGLVTPVAQGMGSGAHTADPEKRKLIQQQLVLLLHAHKCQRREQANGEVRQCNLPHCRTMKNVLNHMTHCQSGKSCQVAHCASSRQIISHWKNCTRHDCPVCLPLKNAGDKRNQQPILTGAPVGLGNPSSLGVGQQSAPNLSTVSQIDPSSIERAYAALGLPYQVNQMPTQPQVQAKNQQNQQPGQSPQGMRPMSNMSASPMGVNGGVGVQTPSLLSDSMLHSAINSQNPMMSENASVPSLGPMPTAAQPSTTGIRKQWHEDITQDLRNHLVHKLVQAIFPTPDPAALKDRRMENLVAYARKVEGDMYESANNRAEYYHLLAEKIYKIQKELEEKRRTRLQKQNMLPNAAGMVPVSMNPGPNMGQPQPGMTSNGPLPDPSMIRGSVPNQMMPRITPQSGLNQFGQMSMAQPPIVPRQTPPLQHHGQLAQPGALNPPMGYGPRMQQPSNQGQFLPQTQFPSQGMNVTNIPLAPSSGQAPVSQAQMSSSSCPVNSPIMPPGSQGSHIHCPQLPQPALHQNSPSPVPSRTPTPHHTPPSIGAQQPPATTIPAPVPTPPAMPPGPQSQALHPPPRQTPTPPTTQLPQQVQPSLPAAPSADQPQQQPRSQQSTAASVPTPTAPLLPPQPATPLSQPAVSIEGQVSNPPSTSSTEVNSQAIAEKQPSQEVKMEAKMEVDQPEPADTQPEDISESKVEDCKMESTETEERSTELKTEIKEEEDQPSTSATQSSPAPGQSKKKIFKPEELRQALMPTLEALYRQDPESLPFRQPVDPQLLGIPDYFDIVKSPMDLSTIKRKLDTGQYQEPWQYVDDIWLMFNNAWLYNRKTSRVYKYCSKLSEVFEQEIDPVMQSLGYCCGRKLEFSPQTLCCYGKQLCTIPRDATYYSYQNRYHFCEKCFNEIQGESVSLGDDPSQPQTTINKEQFSKRKNDTLDPELFVECTECGRKMHQICVLHHEIIWPAGFVCDGCLKKSARTRKENKFSAKRLPSTRLGTFLENRVNDFLRRQNHPESGEVTVRVVHASDKTVEVKPGMKARFVDSGEMAESFPYRTKALFAFEEIDGVDLCFFGMHVQEYGSDCPPPNQRRVYISYLDSVHFFRPKCLRTAVYHEILIGYLEYVKKLGYTTGHIWACPPSEGDDYIFHCHPPDQKIPKPKRLQEWYKKMLDKAVSERIVHDYKDIFKQATEDRLTSAKELPYFEGDFWPNVLEESIKELEQEEEERKREENTSNESTDVTKGDSKNAKKKNNKKTSKNKSSLSRGNKKKPGMPNVSNDLSQKLYATMEKHKEVFFVIRLIAGPAANSLPPIVDPDPLIPCDLMDGRDAFLTLARDKHLEFSSLRRAQWSTMCMLVELHTQSQDRFVYTCNECKHHVETRWHCTVCEDYDLCITCYNTKNHDHKMEKLGLGLDDESNNQQAAATQSPGDSRRLSIQRCIQSLVHACQCRNANCSLPSCQKMKRVVQHTKGCKRKTNGGCPICKQLIALCCYHAKHCQENKCPVPFCLNIKQKLRQQQLQHRLQQAQMLRRRMASMQRTGVVGQQQGLPSPTPATPTTPTGQQPTTPQTPQPTSQPQPTPPNSMPPYLPRTQAAGPVSQGKAAGQVTPPTPPQTAQPPLPGPPPAAVEMAMQIQRAAETQRQMAHVQIFQRPIQHQMPPMTPMAPMGMNPPPMTRGPSGHLEPGMGPTGMQQQPPWSQGGLPQPQQLQSGMPRPAMMSVAQHGQPLNMAPQPGLGQVGISPLKPGTVSQQALQNLLRTLRSPSSPLQQQQVLSILHANPQLLAAFIKQRAAKYANSNPQPIPGQPGMPQGQPGLQPPTMPGQQGVHSNPAMQNMNPMQAGVQRAGLPQQQPQQQLQPPMGGMSPQAQQMNMNHNTMPSQFRDILRRQQMMQQQQQQGAGPGIGPGMANHNQFQQPQGVGYPPQQQQRMQHHMQQMQQGNMGQIGQLPQALGAEAGASLQAYQQRLLQQQMGSPVQPNPMSPQQHMLPNQAQSPHLQGQQIPNSLSNQVRSPQPVPSPRPQSQPPHSSPSPRMQPQPSPHHVSPQTSSPHPGLVAAQANPMEQGHFASPDQNSMLSQLASNPGMANLHGASATDLGLSTDNSDLNSNLSQSTLDIH

SEQ ID NO: 21 - protein, 695 aa, Homo sapiens
MGQTGKKSEKGPVCWRKRVKSEYMRLRQLKRFRRADEVKSMFSSNRQKILERTEILNQEWKQRRIQPVHILTSCSVTSDLDFPTQVIPLKTLNAVASVPIMYSWSPLQQNFMVEDETVLHNIPYMGDEVLDQDGTFIEELIKNYDGKVHGDRECGFINDEIFVELVNALGQYNDDDDDDDGDDPEEREEKQKDLEDHRDDKESRPPRKFPSDKIFEAISSMFPDKGTAEELKEKYKELTEQQLPGALPPECTPNIDGPNAKSVQREQSLHSFHTLFCRRCFKYDCFLHPFHATPNTYKRKNTETALDNKPCGPQCYQHLEGAKEFAAALTAERIKTPPKRPGGRRRGRLPNNSSRPSTPTINVLESKDTDSDREAGTETGGENNDKEEEEKKDETSSSSEANSRCQTPIKMKPNIEPPENVEWSGAEASMFRVLIGTYYDNFCAIARLIGTKTCRQVYEFRVKESSIIAPAPAEDVDTPPRKKKRKHRLWAAHCRKIQLKKGQNRFPGCRCKAQCNTKQCPCYLAVRECDPDLCLTCGAADHWDSKNVSCKNCSIQRGSKKHLLLAPSDVAGWGIFIKDPVQKNEFISEYCGEIISQDEADRRGKVYDKYMCSFLFNLNNDFVVDATRKGNKIRFANHSVNPNCYAKVMMVNGDHRIGIFAKRAIQTGEELFFDYRYSQADALKYVGIEREMEIP

SEQ ID NO: 22 - DNA, 2682 bp, Homo sapiens
gacgacgttcgcggcggggaactcggagtagcttcgcctctgacgtttccccacgacgcaccccgaaatccccctgagctccggcggtcgcgggctgccctcgccgcctggtctggctttatgctaagtttgagggaagagtcgagctgctctgctctctattgattgtgtttctggagggcgtcctgttgaattcccacttcattgtgtacatccccttccgttccccccaaaaatctgtgccacagggttactttttgaaagcgggaggaatcgagaagcacgatcttttggaaaacttggtgaacgcctaaataatcatgggccagactgggaagaaatctgagaagggaccagtttgttggcggaagcgtgtaaaatcagagtacatgcgactgagacagctcaagaggttcagacgagctgatgaagtaaagagtatgtttagttccaatcgtcagaaaattttggaaagaacggaaatcttaaaccaagaatggaaacagcgaaggatacagcctgtgcacatcctgacttcttgttcggtgaccagtgacttggattttccaacacaagtcatcccattaaagactctgaatgcagttgcttcagtacccataatgtattcttggtctcccctacagcagaattttatggtggaagatgaaactgttttacataacattccttatatgggagatgaagttttagatcaggatggtactttcattgaagaactaataaaaaattatgatgggaaagtacacggggatagagaatgtgggtttataaatgatgaaatttttgtggagttggtgaatgcccttggtcaatataatgatgatgacgatgatgatgatggagacgatcctgaagaaagagaagaaaagcagaaagatctggaggatcaccgagatgataaagaaagccgcccacctcggaaatttccttctgataaaatttttgaagccatttcctcaatgtttccagataagggcacagcagaagaactaaaggaaaaatataaagaactcaccgaacagcagctcccaggcgcacttcctcctgaatgtacccccaacatagatggaccaaatgctaaatctgttcagagagagcaaagcttacactcctttcatacgcttttctgtaggcgatgttttaaatatgactgcttcctacatccttttcatgcaacacccaacacttataagcggaagaacacagaaacagctctagacaacaaaccttgtggaccacagtgttaccagcatttggagggagcaaaggagtttgctgctgctctcaccgctgagcggataaagaccccaccaaaacgtccaggaggccgcagaagaggacggcttcccaataacagtagcaggcccagcacccccaccattaatgtgctggaatcaaaggatacagacagtgatagggaagcagggactgaaacggggggagagaacaatgataaagaagaagaagagaagaaagatgaaacttcgagctcctctgaagcaaattctcggtgtcaaacaccaataaagatgaagccaaatattgaacctcctgagaatgtggagtggagtggtgctgaagcctcaatgtttagagtcctcattggcacttactatgacaatttctgtgccattgctaggttaattgggaccaaaacatgtagacaggtgtatgagtttagagtcaaagaatctagcatcatagctccagctcccgctgaggatgtggatactcctccaaggaaaaagaagaggaaacaccggttgtgggctgcacactgcagaaagatacagctgaaaaagggtcaaaaccgctttccgggatgccgctgcaaagcacagtgcaacaccaagcagtgcccgtgctacctggctgtccgagagtgtgaccctgacctctgtcttacttgtggagccgctgaccattgggacagtaaaaatgtgtcctgcaagaactgcagtattcagcggggctccaaaaagcatctattgctggcaccatctgacgtggcaggctgggggatttttatcaaagatcctgtgcagaaaaatgaattcatctcagaatactgtggagagattatttctcaagatgaagctgacagaagagggaaagtgtatgataaatacatgtgcagctttctgttcaacttgaacaatgattttgtggtggatgcaacccgcaagggtaacaaaattcgttttgcaaatcattcggtaaatccaaactgctatgcaaaagttatgatggttaacggtgatcacaggataggtatttttgccaagagagccatccagactggcgaagagctgttttttgattacagatacagccaggctgatgccctgaagtatgtcggcatcgaaagagaaatggaaatcccttgacatctgctacctcctcccccctcctctgaaacagctgccttagcttcaggaacctcgagtactgtgggcaatttagaaaaagaacatgcagtttgaaattctgaatttgcaaagtactgtaagaataatttatagtaatgagtttaaaaatcaactttttattgccttctcaccagctgcaaagtgttttgtaccagtgaatttttgcaataatgcagtatggtacatttttcaactttgaataaagaatacttgaacttgtccttgttgaatc

SEQ ID NO: 23 - DNA, 10197 bp, Homo sapiens
ctgcggggcgctgttgctgtggctgagatttggccgccgcctcccccacccggcctgcgccctccctctccctcggcgcccgcccgcccgctcgcggcccgcgctcgctcctctccctcgcagccggcagggcccccgacccccgtccgggccctcgccggcccggccgcccgtgcccggggctgttttcgcgagcaggtgaaaatggctgagaacttgctggacggaccgcccaaccccaaaagagccaaactcagctcgcccggtttctcggcgaatgacagcacagattttggatcattgtttgacttggaaaatgatcttcctgatgagctgatacccaatggaggagaattaggccttttaaacagtgggaaccttgttccagatgctgcttccaaacataaacaactgtcggagcttctacgaggaggcagcggctctagtatcaacccaggaataggaaatgtgagcgccagcagccccgtgcagcagggcctgggtggccaggctcaagggcagccgaacagtgctaacatggccagcctcagtgccatgggcaagagccctctgagccagggagattcttcagcccccagcctgcctaaacaggcagccagcacctctgggcccacccccgctgcctcccaagcactgaatccgcaagcacaaaagcaagtggggctggcgactagcagccctgccacgtcacagactggacctggtatctgcatgaatgctaactttaaccagacccacccaggcctcctcaatagtaactctggccatagcttaattaatcaggcttcacaagggcaggcgcaagtcatgaatggatctcttggggctgctggcagaggaaggggagctggaatgccgtaccctactccagccatgcagggcgcctcgagcagcgtgctggctgagaccctaacgcaggtttccccgcaaatgactggtcacgcgggactgaacaccgcacaggcaggaggcatggccaagatgggaataactgggaacacaagtccatttggacagccctttagtcaagctggagggcagccaatgggagccactggagtgaacccccagttagccagcaaacagagcatggtcaacagtttgcccaccttccctacagatatcaagaatacttcagtcaccaacgtgccaaatatgtctcagatgcaaacatcagtgggaattgtacccacacaagcaattgcaacaggccccactgcagatcctgaaaaacgcaaactgatacagcagcagctggttctactgcttcatgctcataagtgtcagagacgagagcaagcaaacggagaggttcgggcctgctcgctcccgcattgtcgaaccatgaaaaacgttttgaatcacatgacgcattgtcaggctgggaaagcctgccaagttgcccattgtgcatcttcacgacaaatcatctctcattggaagaactgcacacgacatgactgtcctgtttgcctccctttgaaaaatgccagtgacaagcgaaaccaacaaaccatcctggggtctccagctagtggaattcaaaacacaattggttctgttggcacagggcaacagaatgccacttctttaagtaacccaaatcccatagaccccagctccatgcagcgagcctatgctgctctcggactcccctacatgaaccagccccagacgcagctgcagcctcaggttcctggccagcaaccagcacagcctcaaacccaccagcagatgaggactctcaaccccctgggaaataatccaatgaacattccagcaggaggaataacaacagatcagcagcccccaaacttgatttcagaatcagctcttccgacttccctgggggccacaaacccactgatgaacgatggctccaactctggtaacattggaaccctcagcactataccaacagcagctcctccttctagcaccggtgtaaggaaaggctggcacgaacatgtcactcaggacctgcggagccatctagtgcataaactcgtccaagccatcttcccaacacctgatcccgcagctctaaaggatcgccgcatggaaaacctggtagcctatgctaagaaagtggaaggggacatgtacgagtctgccaacagcagggatgaatattatcacttattagcagagaaaatctacaagatacaaaaagaactagaagaaaaacggaggtcgcgtttacataaacaaggcatcttggggaaccagccagccttaccagccccgggggctcagccccctgtgattccacaggcacaacctgtgagacctccaaatggacccctgtccctgccagtgaatcgcatgcaagtttctcaagggatgaattcatttaaccccatgtccttggggaacgtccagttgccacaagcacccatgggacctcgtgcagcctccccaatgaaccactctgtccagatgaacagcatgggctcagtgccagggatggccatttctccttcccgaatgcctcagcctccgaacatgatgggtgcacacaccaacaacatgatggcccaggcgcccgctcagagccagtttctgccacagaaccagttcccgtcatccagcggggcgatgagtgtgggcatggggcagccgccagcccaaacaggcgtgtcacagggacaggtgcctggtgctgctcttcctaaccctctcaacatgctggggcctcaggccagccagctaccttgccctccagtgacacagtcaccactgcacccaacaccgcctcctgcttccacggctgctggcatgccatctctccagcacacgacaccacctgggatgactcctccccagccagcagctcccactcagccatcaactcctgtgtcgtcttccgggcagactcccaccccgactcctggctcagtgcccagtgctacccaaacccagagcacccctacagtccaggcagcagcccaggcccaggtgaccccgcagcctcaaaccccagttcagcccccgtctgtggctacccctcagtcatcgcagcaacagccgacgcctgtgcacgcccagcctcctggcacaccgctttcccaggcagcagccagcattgataacagagtccctaccccctcctcggtggccagcgcagaaaccaattcccagcagccaggacctgacgtacctgtgctggaaatgaagacggagacccaagcagaggacactgagcccgatcctggtgaatccaaaggggagcccaggtctgagatgatggaggaggatttgcaaggagcttcccaagttaaagaagaaacagacatagcagagcagaaatcagaaccaatggaagtggatgaaaagaaacctgaagtgaaagtagaagttaaagaggaagaagagagtagcagtaacggcacagcctctcagtcaacatctccttcgcagccgcgcaaaaaaatctttaaaccagaggagttacgccaggccctcatgccaaccctagaagcactgtatcgacaggacccagagtcattacctttccggcagcctgtagatccccagctcctcggaattccagactattttgacatcgtaaagaatcccatggacctctccaccatcaagcggaagctggacacagggcaataccaagagccctggcagtacgtggacgacgtctggctcatgttcaacaatgcctggctctataatcgcaagacatcccgagtctataagttttgcagtaagcttgcagaggtctttgagcaggaaattgaccctgtcatgcagtcccttggatattgctgtggacgcaagtatgagttttccccacagactttgtgctgctatgggaagcagctgtgtaccattcctcgcgatgctgcctactacagctatcagaataggtatcatttctgtgagaagtgtttcacagagatccagggcgagaatgtgaccctgggtgacgacccttcacagccccagacgacaatttcaaaggatcagtttgaaaagaagaaaaatgataccttagaccccgaacctttcgttgattgcaaggagtgtggccggaagatgcatcagatttgcgttctgcactatgacatcatttggccttcaggttttgtgtgcgacaactgcttgaagaaaactggcagacctcgaaaagaaaacaaattcagtgctaagaggctgcagaccacaagactgggaaaccacttggaagaccgagtgaacaaatttttgcggcgccagaatcaccctgaagccggggaggtttttgtccgagtggtggccagctcagacaagacggtggaggtcaagcccgggatgaagtcacggtttgtggattctggggaaatgtctgaatctttcccatatcgaaccaaagctctgtttgcttttgaggaaattgacggcgtggatgtctgcttttttggaatgcacgtccaagaatacggctctgattgcccccctccaaacacgaggcgtgtgtacatttcttatctggatagtattcatttcttccggccacgttgcctccgcacagccgtttaccatgagatccttattggatatttagagtatgtgaagaaattagggtatgtgacagggcacatctgggcctgtcctccaagtgaaggagatgattacatcttccattgccacccacctgatcaaaaaatacccaagccaaaacgactgcaggagtggtacaaaaagatgctggacaaggcgtttgcagagcggatcatccatgactacaaggatattttcaaacaagcaactgaagacaggctcaccagtgccaaggaactgccctattttgaaggtgatttctggcccaatgtgttagaagagagcattaaggaactagaacaagaagaagaggagaggaaaaaggaagagagcactgcagccagtgaaaccactgagggcagtcagggcgacagcaagaatgccaagaagaagaacaacaagaaaaccaacaagaacaaaagcagcatcagccgcgccaacaagaagaagcccagcatgcccaacgtgtccaatgacctgtcccagaagctgtatgccaccatggagaagcacaaggaggtcttcttcgtgatccacctgcacgctgggcctgtcatcaacaccctgccccccatcgtcgaccccgaccccctgctcagctgtgacctcatggatgggcgcgacgccttcctcaccctcgccagagacaagcactgggagttctcctccttgcgccgctccaagtggtccacgctctgcatgctggtggagctgcacacccagggccaggaccgctttgtctacacctgcaacgagtgcaagcaccacgtggagacgcgctggcactgcactgtgtgcgaggactacgacctctgcatcaactgctataacacgaagagccatgcccataagatggtgaagtgggggctgggcctggatgacgagggcagcagccagggcgagccacagtcaaagagcccccaggagtcacgccggctgagcatccagcgctgcatccagtcgctggtgcacgcgtgccagtgccgcaacgccaactgctcgctgccatcctgccagaagatgaagcgggtggtgcagcacaccaagggctgcaaacgcaagaccaacgggggctgcccggtgtgcaagcagctcatcgccctctgctgctaccacgccaagcactgccaagaaaacaaatgccccgtgcccttctgcctcaacatcaaacacaagctccgccagcagcagatccagcaccgcctgcagcaggcccagctcatgcgccggcggatggccaccatgaacacccgcaacgtgcctcagcagagtctgccttctcctacctcagcaccgcccgggacccccacacagcagcccagcacaccccagacgccgcagccccctgcccagccccaaccctcacccgtgagcatgtcaccagctggcttccccagcgtggcccggactcagccccccaccacggtgtccacagggaagcctaccagccaggtgccggcccccccacccccggcccagccccctcctgcagcggtggaagcggctcggcagatcgagcgtgaggcccagcagcagcagcacctgtaccgggtgaacatcaacaacagcatgcccccaggacgcacgggcatggggaccccggggagccagatggcccccgtgagcctgaatgtgccccgacccaaccaggtgagcgggcccgtcatgcccagcatgcctcccgggcagtggcagcaggcgccccttccccagcagcagcccatgccaggcttgcccaggcctgtgatatccatgcaggcccaggcggccgtggctgggccccggatgcccagcgtgcagccacccaggagcatctcacccagcgctctgcaagacctgctgcggaccctgaagtcgcccagctcccctcagcagcaacagcaggtgctgaacattctcaaatcaaacccgcagctaatggcagctttcatcaaacagcgcacagccaagtacgtggccaatcagcccggcatgcagccccagcctggcctccagtcccagcccggcatgcaaccccagcctggcatgcaccagcagcccagcctgcagaacctgaatgccatgcaggctggcgtgccgcggcccggtgtgcctccacagcagcaggcgatgggaggcctgaacccccagggccaggccttgaacatcatgaacccaggacacaaccccaacatggcgagtatgaatccacagtaccgagaaatgttacggaggcagctgctgcagcagcagcagcaacagcagcagcaacaacagcagcaacagcagcagcagcaagggagtgccggcatggctgggggcatggcggggcacggccagttccagcagcctcaaggacccggaggctacccaccggccatgcagcagcagcagcgcatgcagcagcatctccccctccagggcagctccatgggccagatggcggctcagatgggacagcttggccagatggggcagccggggctgggggcagacagcacccccaacatccagcaagccctgcagcagcggattctgcagcaacagcagatgaagcagcagattgggtccccaggccagccgaaccccatgagcccccagcaacacatgctctcaggacagccacaggcctcgcatctccctggccagcagatcgccacgtcccttagtaaccaggtgcggtctccagcccctgtccagtctccacggccccagtcccagcctccacattccagcccgtcaccacggatacagccccagccttcgccacaccacgtctcaccccagactggttccccccaccccggactcgcagtcaccatggccagctccatagatcagggacacttggggaaccccgaacagagtgcaatgctcccccagctgaacacccccagcaggagtgcgctgtccagcgaactgtccctggtcggggacaccacgggggacacgctagagaagtttgtggagggcttgtagcattgtgagagcatcaccttttccctttcatgttcttggaccttttgtactgaaaatccaggcatctaggttctttttattcctagatggaactgcgacttccgagccatggaagggtggattgatgtttaaagaaacaatacaaagaatatatttttttgttaaaaaccagttgatttaaatatctggtctctctctttggtttttttttggcgggggggtggggggggttcttttttttccgttttgtttttgtttggggggaggggggttttgtttggattctttttgtcgtcattgctggtgactcatgcctttttttaacgggaaaaacaagttcattatattcatattttttatttgtattttcaagactttaaacatttatgtttaaaagtaagaagaaaaataatattcagaactgattcctgaaataatgcaagcttataatgtatcccgataactttgtgatgtttcgggaagatttttttctatagtgaactctgtgggcgtctcccagtattaccctggatgataggaattgactccggcgtgcacacacgtacacacccacacacatctatctatacataatggctgaagccaaacttgtcttgcagatgtagaaattgttgctttgtttctctgataaaactggttttagacaaaaaatagggatgatcactcttagaccatgctaatgttactagagaagaagccttcttttctttcttctatgtgaaacttgaaatgaggaaaagcaattctagtgtaaatcatgcaagcgctctaattcctataaatacgaaactcgagaagattcaatcactgtatagaatggtaaaataccaactcatttcttatatcatattgttaaataaactgtgtgcaacagacaaaaagggtggtccttcttgaattcatgtacatggtattaacacttagtgttcggggttttttgttatgaaaatgctgttttcaacattgtatttggactatgcatgtgttttttccccattgtatataaagtaccgcttaaaattgatataaattactgaggtttttaacatgtattctgttctttaagatccctgtaagaatgtttaaggtttttatttatttatatatattttttgagtctgttctttgtaagacatggttctggttgttcgctcatagcggagaggctggggctgcggttgtggttgtggcggcgtgggtggtggctgggaactgtggcccaggcttagcggccgcccggaggcttttcttcccggagactgaggtgggcgactgaggtgggcggctcagcgttggccccacacattcgaggctcacaggtgattgtcgctcacacagttagggtcgtcagttggtctgaaactgcatttggcccactcctccatcctccctgtccgtcgtagctgccacccccagaggcggcgcttcttcccgtgttcaggcggctccccccccccgtacacgactcccagaatctgaggcagagagtgctccaggctcgcgaggtgctttctgacttccccccaaatcctgccgctgccgcgcagcatgtcccgtgtggcgtttgaggaaatgctgagggacagacaccttggagcaccagctccggtccctgttacagtgagaaaggtcccccacttcgggggatacttgcacttagccacatggtcctgcctcccttggagtccagttccaggctcccttactgagtgggtgagacaagttcacaaaaaccgtaaaactgagaggaggaccatgggcaggggagctgaagttcatcccctaagtctaccacccccagcacccagagaacccactttatccctagtcccccaacaaaggctggtctaggtgggggtgatggtaattttagaaatcacgccccaaatagcttccgtttgggcccttacattcacagataggttttaaatagctgaatacttggtttgggaatctgaattcgaggaacctttctaagaagttggaaaggtccgatctagttttagcacagagctttgaaccttgagttataaaatgcagaataattcaagtaaaaataagaccaccatctggcacccctgaccagcccccattcaccccatcccaggaggggaagcacaggccgggcctccggtggagattgctgccactgctcggcctgctgggttcttaacctccagtgtcctcttcatcttttccacccgtagggaaaccttgagccatgtgttcaaacaagaagtggggctagagcccgagagcagcagctctaagcccacactcagaaagtggcgccctcctggttgtgcagccttttaatgtgggcagtggaggggcctctgtttcaggttatcctggaattcaaaacgttatgtaccaacctcatcctctttggagtctgcatcctgtgcaaccgtcttgggcaatccagatgtcgaaggatgtgaccgagagcatggtctgtggatgctaaccctaagtttgtcgtaaggaaatttctgtaagaaacctggaaagccccaacgctgtgtctcatgctgtatacttaagaggagaagaaaaagtcctatatttgtgatcaaaaagaggaaacttgaaatgtgatggtgtttataataaaagatggtaaaactacttggattcaaa

SEQ ID NO: 24 - protein, 2442 aa, Homo sapiens
MAENLLDGPPNPKRAKLSSPGFSANDSTDFGSLFDLENDLPDELIPNGGELGLLNSGNLVPDAASKHKQLSELLRGGSGSSINPGIGNVSASSPVQQGLGGQAQGQPNSANMASLSAMGKSPLSQGDSSAPSLPKQAASTSGPTPAASQALNPQAQKQVGLATSSPATSQTGPGICMNANFNQTHPGLLNSNSGHSLINQASQGQAQVMNGSLGAAGRGRGAGMPYPTPAMQGASSSVLAETLTQVSPQMTGHAGLNTAQAGGMAKMGITGNTSPFGQPFSQAGGQPMGATGVNPQLASKQSMVNSLPTFPTDIKNTSVTNVPNMSQMQTSVGIVPTQAIATGPTADPEKRKLIQQQLVLLLHAHKCQRREQANGEVRACSLPHCRTMKNVLNHMTHCQAGKACQVAHCASSRQIISHWKNCTRHDCPVCLPLKNASDKRNQQTILGSPASGIQNTIGSVGTGQQNATSLSNPNPIDPSSMQRAYAALGLPYMNQPQTQLQPQVPGQQPAQPQTHQQMRTLNPLGNNPMNIPAGGITTDQQPPNLISESALPTSLGATNPLMNDGSNSGNIGTLSTIPTAAPPSSTGVRKGWHEHVTQDLRSHLVHKLVQAIFPTPDPAALKDRRMENLVAYAKKVEGDMYESANSRDEYYHLLAEKIYKIQKELEEKRRSRLHKQGILGNQPALPAPGAQPPVIPQAQPVRPPNGPLSLPVNRMQVSQGMNSFNPMSLGNVQLPQAPMGPRAASPMNHSVQMNSMGSVPGMAISPSRMPQPPNMMGAHTNNMMAQAPAQSQFLPQNQFPSSSGAMSVGMGQPPAQTGVSQGQVPGAALPNPLNMLGPQASQLPCPPVTQSPLHPTPPPASTAAGMPSLQHTTPPGMTPPQPAAPTQPSTPVSSSGQTPTPTPGSVPSATQTQSTPTVQAAAQAQVTPQPQTPVQPPSVATPQSSQQQPTPVHAQPPGTPLSQAAASIDNRVPTPSSVASAETNSQQPGPDVPVLEMKTETQAEDTEPDPGESKGEPRSEMMEEDLQGASQVKEETDIAEQKSEPMEVDEKKPEVKVEVKEEEESSSNGTASQSTSPSQPRKKIFKPEELRQALMPTLEALYRQDPESLPFRQPVDPQLLGIPDYFDIVKNPMDLSTIKRKLDTGQYQEPWQYVDDVWLMFNNAWLYNRKTSRVYKFCSKLAEVFEQEIDPVMQSLGYCCGRKYEFSPQTLCCYGKQLCTIPRDAAYYSYQNRYHFCEKCFTEIQGENVTLGDDPSQPQTTISKDQFEKKKNDTLDPEPFVDCKECGRKMHQICVLHYDIIWPSGFVCDNCLKKTGRPRKENKFSAKRLQTTRLGNHLEDRVNKFLRRQNHPEAGEVFVRVVASSDKTVEVKPGMKSRFVDSGEMSESFPYRTKALFAFEEIDGVDVCFFGMHVQEYGSDCPPPNTRRVYISYLDSIHFFRPRCLRTAVYHEILIGYLEYVKKLGYVTGHIWACPPSEGDDYIFHCHPPDQKIPKPKRLQEWYKKMLDKAFAERIIHDYKDIFKQATEDRLTSAKELPYFEGDFWPNVLEESIKELEQEEEERKKEESTAASETTEGSQGDSKNAKKKNNKKTNKNKSSISRANKKKPSMPNVSNDLSQKLYATMEKHKEVFFVIHLHAGPVINTLPPIVDPDPLLSCDLMDGRDAFLTLARDKHWEFSSLRRSKWSTLCMLVELHTQGQDRFVYTCNECKHHVETRWHCTVCEDYDLCINCYNTKSHAHKMVKWGLGLDDEGSSQGEPQSKSPQESRRLSIQRCIQSLVHACQCRNANCSLPSCQKMKRVVQHTKGCKRKTNGGCPVCKQLIALCCYHAKHCQENKCPVPFCLNIKHKLRQQQIQHRLQQAQLMRRRMATMNTRNVPQQSLPSPTSAPPGTPTQQPSTPQTPQPPAQPQPSPVSMSPAGFPSVARTQPPTTVSTGKPTSQVPAPPPPAQPPPAAVEAARQIEREAQQQQHLYRVNINNSMPPGRTGMGTPGSQMAPVSLNVPRPNQVSGPVMPSMPPGQWQQAPLPQQQPMPGLPRPVISMQAQAAVAGPRMPSVQPPRSISPSALQDLLRTLKSPSSPQQQQQVLNILKSNPQLMAAFIKQRTAKYVANQPGMQPQPGLQSQPGMQPQPGMHQQPSLQNLNAMQAGVPRPGVPPQQQAMGGLNPQGQALNIMNPGHNPNMASMNPQYREMLRRQLLQQQQQQQQQQQQQQQQQQGSAGMAGGMAGHGQFQQPQGPGGYPPAMQQQQRMQQHLPLQGSSMGQMAAQMGQLGQMGQPGLGADSTPNIQQALQQRILQQQQMKQQIGSPGQPNPMSPQQHMLSGQPQASHLPGQQIATSLSNQVRSPAPVQSPRPQSQPPHSSPSPRIQPQPSPHHVSPQTGSPHPGLAVTMASSIDQGHLGNPEQSAMLPQLNTPSRSALSSELSLVGDTTGDTLEKFVEGL

SEQ ID NO: 25 - DNA, 10083 bp, Homo sapiens
ctgcggggcgctgttgctgtggctgagatttggccgccgcctcccccacccggcctgcgccctccctctccctcggcgcccgcccgcccgctcgcggcccgcgctcgctcctctccctcgcagccggcagggcccccgacccccgtccgggccctcgccggcccggccgcccgtgcccggggctgttttcgcgagcaggtgaaaatggctgagaacttgctggacggaccgcccaaccccaaaagagccaaactcagctcgcccggtttctcggcgaatgacagcacagattttggatcattgtttgacttggaaaatgatcttcctgatgagctgatacccaatggaggagaattaggccttttaaacagtgggaaccttgttccagatgctgcttccaaacataaacaactgtcggagcttctacgaggaggcagcggctctagtatcaacccaggaataggaaatgtgagcgccagcagccccgtgcagcagggcctgggtggccaggctcaagggcagccgaacagtgctaacatggccagcctcagtgccatgggcaagagccctctgagccagggagattcttcagcccccagcctgcctaaacaggcagccagcacctctgggcccacccccgctgcctcccaagcactgaatccgcaagcacaaaagcaagtggggctggcgactagcagccctgccacgtcacagactggacctggtatctgcatgaatgctaactttaaccagacccacccaggcctcctcaatagtaactctggccatagcttaattaatcaggcttcacaagggcaggcgcaagtcatgaatggatctcttggggctgctggcagaggaaggggagctggaatgccgtaccctactccagccatgcagggcgcctcgagcagcgtgctggctgagaccctaacgcaggtttccccgcaaatgactggtcacgcgggactgaacaccgcacaggcaggaggcatggccaagatgggaataactgggaacacaagtccatttggacagccctttagtcaagctggagggcagccaatgggagccactggagtgaacccccagttagccagcaaacagagcatggtcaacagtttgcccaccttccctacagatatcaagaatacttcagtcaccaacgtgccaaatatgtctcagatgcaaacatcagtgggaattgtacccacacaagcaattgcaacaggccccactgcagatcctgaaaaacgcaaactgatacagcagcagctggttctactgcttcatgctcataagtgtcagagacgagagcaagcaaacggagaggttcgggcctgctcgctcccgcattgtcgaaccatgaaaaacgttttgaatcacatgacgcattgtcaggctgggaaagcctgccaagccatcctggggtctccagctagtggaattcaaaacacaattggttctgttggcacagggcaacagaatgccacttctttaagtaacccaaatcccatagaccccagctccatgcagcgagcctatgctgctctcggactcccctacatgaaccagccccagacgcagctgcagcctcaggttcctggccagcaaccagcacagcctcaaacccaccagcagatgaggactctcaaccccctgggaaataatccaatgaacattccagcaggaggaataacaacagatcagcagcccccaaacttgatttcagaatcagctcttccgacttccctgggggccacaaacccactgatgaacgatggctccaactctggtaacattggaaccctcagcactataccaacagcagctcctccttctagcaccggtgtaaggaaaggctggcacgaacatgtcactcaggacctgcggagccatctagtgcataaactcgtccaagccatcttcccaacacctgatcccgcagctctaaaggatcgccgcatggaaaacctggtagcctatgctaagaaagtggaaggggacatgtacgagtctgccaacagcagggatgaatattatcacttattagcagagaaaatctacaagatacaaaaagaactagaagaaaaacggaggtcgcgtttacataaacaaggcatcttggggaaccagccagccttaccagccccgggggctcagccccctgtgattccacaggcacaacctgtgagacctccaaatggacccctgtccctgccagtgaatcgcatgcaagtttctcaagggatgaattcatttaaccccatgtccttggggaacgtccagttgccacaagcacccatgggacctcgtgcagcctccccaatgaaccactctgtccagatgaacagcatgggctcagtgccagggatggccatttctccttcccgaatgcctcagcctccgaacatgatgggtgcacacaccaacaacatgatggcccaggcgcccgctcagagccagtttctgccacagaaccagttcccgtcatccagcggggcgatgagtgtgggcatggggcagccgccagcccaaacaggcgtgtcacagggacaggtgcctggtgctgctcttcctaaccctctcaacatgctggggcctcaggccagccagctaccttgccctccagtgacacagtcaccactgcacccaacaccgcctcctgcttccacggctgctggcatgccatctctccagcacacgacaccacctgggatgactcctccccagccagcagctcccactcagccatcaactcctgtgtcgtcttccgggcagactcccaccccgactcctggctcagtgcccagtgctacccaaacccagagcacccctacagtccaggcagcagcccaggcccaggtgaccccgcagcctcaaaccccagttcagcccccgtctgtggctacccctcagtcatcgcagcaacagccgacgcctgtgcacgcccagcctcctggcacaccgctttcccaggcagcagccagcattgataacagagtccctaccccctcctcggtggccagcgcagaaaccaattcccagcagccaggacctgacgtacctgtgctggaaatgaagacggagacccaagcagaggacactgagcccgatcctggtgaatccaaaggggagcccaggtctgagatgatggaggaggatttgcaaggagcttcccaagttaaagaagaaacagacatagcagagcagaaatcagaaccaatggaagtggatgaaaagaaacctgaagtgaaagtagaagttaaagaggaagaagagagtagcagtaacggcacagcctctcagtcaacatctccttcgcagccgcgcaaaaaaatctttaaaccagaggagttacgccaggccctcatgccaaccctagaagcactgtatcgacaggacccagagtcattacctttccggcagcctgtagatccccagctcctcggaattccagactattttgacatcgtaaagaatcccatggacctctccaccatcaagcggaagctggacacagggcaataccaagagccctggcagtacgtggacgacgtctggctcatgttcaacaatgcctggctctataatcgcaagacatcccgagtctataagttttgcagtaagcttgcagaggtctttgagcaggaaattgaccctgtcatgcagtcccttggatattgctgtggacgcaagtatgagttttccccacagactttgtgctgctatgggaagcagctgtgtaccattcctcgcgatgctgcctactacagctatcagaataggtatcatttctgtgagaagtgtttcacagagatccagggcgagaatgtgaccctgggtgacgacccttcacagccccagacgacaatttcaaaggatcagtttgaaaagaagaaaaatgataccttagaccccgaacctttcgttgattgcaaggagtgtggccggaagatgcatcagatttgcgttctgcactatgacatcatttggccttcaggttttgtgtgcgacaactgcttgaagaaaactggcagacctcgaaaagaaaacaaattcagtgctaagaggctgcagaccacaagactgggaaaccacttggaagaccgagtgaacaaatttttgcggcgccagaatcaccctgaagccggggaggtttttgtccgagtggtggccagctcagacaagacggtggaggtcaagcccgggatgaagtcacggtttgtggattctggggaaatgtctgaatctttcccatatcgaaccaaagctctgtttgcttttgaggaaattgacggcgtggatgtctgcttttttggaatgcacgtccaagaatacggctctgattgcccccctccaaacacgaggcgtgtgtacatttcttatctggatagtattcatttcttccggccacgttgcctccgcacagccgtttaccatgagatccttattggatatttagagtatgtgaagaaattagggtatgtgacagggcacatctgggcctgtcctccaagtgaaggagatgattacatcttccattgccacccacctgatcaaaaaatacccaagccaaaacgactgcaggagtggtacaaaaagatgctggacaaggcgtttgcagagcggatcatccatgactacaaggatattttcaaacaagcaactgaagacaggctcaccagtgccaaggaactgccctattttgaaggtgatttctggcccaatgtgttagaagagagcattaaggaactagaacaagaagaagaggagaggaaaaaggaagagagcactgcagccagtgaaaccactgagggcagtcagggcgacagcaagaatgccaagaagaagaacaacaagaaaaccaacaagaacaaaagcagcatcagccgcgccaacaagaagaagcccagcatgcccaacgtgtccaatgacctgtcccagaagctgtatgccaccatggagaagcacaaggaggtcttcttcgtgatccacctgcacgctgggcctgtcatcaacaccctgccccccatcgtcgaccccgaccccctgctcagctgtgacctcatggatgggcgcgacgccttcctcaccctcgccagagacaagcactgggagttctcctccttgcgccgctccaagtggtccacgctctgcatgctggtggagctgcacacccagggccaggaccgctttgtctacacctgcaacgagtgcaagcaccacgtggagacgcgctggcactgcactgtgtgcgaggactacgacctctgcatcaactgctataacacgaagagccatgcccataagatggtgaagtgggggctgggcctggatgacgagggcagcagccagggcgagccacagtcaaagagcccccaggagtcacgccggctgagcatccagcgctgcatccagtcgctggtgcacgcgtgccagtgccgcaacgccaactgctcgctgccatcctgccagaagatgaagcgggtggtgcagcacaccaagggctgcaaacgcaagaccaacgggggctgcccggtgtgcaagcagctcatcgccctctgctgctaccacgccaagcactgccaagaaaacaaatgccccgtgcccttctgcctcaacatcaaacacaagctccgccagcagcagatccagcaccgcctgcagcaggcccagctcatgcgccggcggatggccaccatgaacacccgcaacgtgcctcagcagagtctgccttctcctacctcagcaccgcccgggacccccacacagcagcccagcacaccccagacgccgcagccccctgcccagccccaaccctcacccgtgagcatgtcaccagctggcttccccagcgtggcccggactcagccccccaccacggtgtccacagggaagcctaccagccaggtgccggcccccccacccccggcccagccccctcctgcagcggtggaagcggctcggcagatcgagcgtgaggcccagcagcagcagcacctgtaccgggtgaacatcaacaacagcatgcccccaggacgcacgggcatggggaccccggggagccagatggcccccgtgagcctgaatgtgccccgacccaaccaggtgagcgggcccgtcatgcccagcatgcctcccgggcagtggcagcaggcgccccttccccagcagcagcccatgccaggcttgcccaggcctgtgatatccatgcaggcccaggcggccgtggctgggccccggatgcccagcgtgcagccacccaggagcatctcacccagcgctctgcaagacctgctgcggaccctgaagtcgcccagctcccctcagcagcaacagcaggtgctgaacattctcaaatcaaacccgcagctaatggcagctttcatcaaacagcgcacagccaagtacgtggccaatcagcccggcatgcagccccagcctggcctccagtcccagcccggcatgcaaccccagcctggcatgcaccagcagcccagcctgcagaacctgaatgccatgcaggctggcgtgccgcggcccggtgtgcctccacagcagcaggcgatgggaggcctgaacccccagggccaggccttgaacatcatgaacccaggacacaaccccaacatggcgagtatgaatccacagtaccgagaaatgttacggaggcagctgctgcagcagcagcagcaacagcagcagcaacaacagcagcaacagcagcagcagcaagggagtgccggcatggctgggggcatggcggggcacggccagttccagcagcctcaaggacccggaggctacccaccggccatgcagcagcagcagcgcatgcagcagcatctccccctccagggcagctccatgggccagatggcggctcagatgggacagcttggccagatggggcagccggggctgggggcagacagcacccccaacatccagcaagccctgcagcagcggattctgcagcaacagcagatgaagcagcagattgggtccccaggccagccgaaccccatgagcccccagcaacacatgctctcaggacagccacaggcctcgcatctccctggccagcagatcgccacgtcccttagtaaccaggtgcggtctccagcccctgtccagtctccacggccccagtcccagcctccacattccagcccgtcaccacggatacagccccagccttcgccacaccacgtctcaccccagactggttccccccaccccggactcgcagtcaccatggccagctccatagatcagggacacttggggaaccccgaacagagtgcaatgctcccccagctgaacacccccagcaggagtgcgctgtccagcgaactgtccctggtcggggacaccacgggggacacgctagagaagtttgtggagggcttgtagcattgtgagagcatcaccttttccctttcatgttcttggaccttttgtactgaaaatccaggcatctaggttctttttattcctagatggaactgcgacttccgagccatggaagggtggattgatgtttaaagaaacaatacaaagaatatatttttttgttaaaaaccagttgatttaaatatctggtctctctctttggtttttttttggcgggggggtggggggggttcttttttttccgttttgtttttgtttggggggaggggggttttgtttggattctttttgtcgtcattgctggtgactcatgcctttttttaacgggaaaaacaagttcattatattcatattttttatttgtattttcaagactttaaacatttatgtttaaaagtaagaagaaaaataatattcagaactgattcctgaaataatgcaagcttataatgtatcccgataactttgtgatgtttcgggaagatttttttctatagtgaactctgtgggcgtctcccagtattaccctggatgataggaattgactccggcgtgcacacacgtacacacccacacacatctatctatacataatggctgaagccaaacttgtcttgcagatgtagaaattgttgctttgtttctctgataaaactggttttagacaaaaaatagggatgatcactcttagaccatgctaatgttactagagaagaagccttcttttctttcttctatgtgaaacttgaaatgaggaaaagcaattctagtgtaaatcatgcaagcgctctaattcctataaatacgaaactcgagaagattcaatcactgtatagaatggtaaaataccaactcatttcttatatcatattgttaaataaactgtgtgcaacagacaaaaagggtggtccttcttgaattcatgtacatggtattaacacttagtgttcggggttttttgttatgaaaatgctgttttcaacattgtatttggactatgcatgtgttttttccccattgtatataaagtaccgcttaaaattgatataaattactgaggtttttaacatgtattctgttctttaagatccctgtaagaatgtttaaggtttttatttatttatatatattttttgagtctgttctttgtaagacatggttctggttgttcgctcatagcggagaggctggggctgcggttgtggttgtggcggcgtgggtggtggctgggaactgtggcccaggcttagcggccgcccggaggcttttcttcccggagactgaggtgggcgactgaggtgggcggctcagcgttggccccacacattcgaggctcacaggtgattgtcgctcacacagttagggtcgtcagttggtctgaaactgcatttggcccactcctccatcctccctgtccgtcgtagctgccacccccagaggcggcgcttcttcccgtgttcaggcggctccccccccccgtacacgactcccagaatctgaggcagagagtgctccaggctcgcgaggtgctttctgacttccccccaaatcctgccgctgccgcgcagcatgtcccgtgtggcgtttgaggaaatgctgagggacagacaccttggagcaccagctccggtccctgttacagtgagaaaggtcccccacttcgggggatacttgcacttagccacatggtcctgcctcccttggagtccagttccaggctcccttactgagtgggtgagacaagttcacaaaaaccgtaaaactgagaggaggaccatgggcaggggagctgaagttcatcccctaagtctaccacccccagcacccagagaacccactttatccctagtcccccaacaaaggctggtctaggtgggggtgatggtaattttagaaatcacgccccaaatagcttccgtttgggcccttacattcacagataggttttaaatagctgaatacttggtttgggaatctgaattcgaggaacctttctaagaagttggaaaggtccgatctagttttagcacagagctttgaaccttgagttataaaatgcagaataattcaagtaaaaataagaccaccatctggcacccctgaccagcccccattcaccccatcccaggaggggaagcacaggccgggcctccggtggagattgctgccactgctcggcctgctgggttcttaacctccagtgtcctcttcatcttttccacccgtagggaaaccttgagccatgtgttcaaacaagaagtggggctagagcccgagagcagcagctctaagcccacactcagaaagtggcgccctcctggttgtgcagccttttaatgtgggcagtggaggggcctctgtttcaggttatcctggaattcaaaacgttatgtaccaacctcatcctctttggagtctgcatcctgtgcaaccgtcttgggcaatccagatgtcgaaggatgtgaccgagagcatggtctgtggatgctaaccctaagtttgtcgtaaggaaatttctgtaagaaacctggaaagccccaacgctgtgtctcatgctgtatacttaagaggagaagaaaaagtcctatatttgtgatcaaaaagaggaaacttgaaatgtgatggtgtttataataaaagatggtaaaactacttggattcaaa

SEQ ID NO: 26 - protein, 2404 aa, Homo sapiens
MAENLLDGPPNPKRAKLSSPGFSANDSTDFGSLFDLENDLPDELIPNGGELGLLNSGNLVPDAASKHKQLSELLRGGSGSSINPGIGNVSASSPVQQGLGGQAQGQPNSANMASLSAMGKSPLSQGDSSAPSLPKQAASTSGPTPAASQALNPQAQKQVGLATSSPATSQTGPGICMNANFNQTHPGLLNSNSGHSLINQASQGQAQVMNGSLGAAGRGRGAGMPYPTPAMQGASSSVLAETLTQVSPQMTGHAGLNTAQAGGMAKMGITGNTSPFGQPFSQAGGQPMGATGVNPQLASKQSMVNSLPTFPTDIKNTSVTNVPNMSQMQTSVGIVPTQAIATGPTADPEKRKLIQQQLVLLLHAHKCQRREQANGEVRACSLPHCRTMKNVLNHMTHCQAGKACQAILGSPASGIQNTIGSVGTGQQNATSLSNPNPIDPSSMQRAYAALGLPYMNQPQTQLQPQVPGQQPAQPQTHQQMRTLNPLGNNPMNIPAGGITTDQQPPNLISESALPTSLGATNPLMNDGSNSGNIGTLSTIPTAAPPSSTGVRKGWHEHVTQDLRSHLVHKLVQAIFPTPDPAALKDRRMENLVAYAKKVEGDMYESANSRDEYYHLLAEKIYKIQKELEEKRRSRLHKQGILGNQPALPAPGAQPPVIPQAQPVRPPNGPLSLPVNRMQVSQGMNSFNPMSLGNVQLPQAPMGPRAASPMNHSVQMNSMGSVPGMAISPSRMPQPPNMMGAHTNNMMAQAPAQSQFLPQNQFPSSSGAMSVGMGQPPAQTGVSQGQVPGAALPNPLNMLGPQASQLPCPPVTQSPLHPTPPPASTAAGMPSLQHTTPPGMTPPQPAAPTQPSTPVSSSGQTPTPTPGSVPSATQTQSTPTVQAAAQAQVTPQPQTPVQPPSVATPQSSQQQPTPVHAQPPGTPLSQAAASIDNRVPTPSSVASAETNSQQPGPDVPVLEMKTETQAEDTEPDPGESKGEPRSEMMEEDLQGASQVKEETDIAEQKSEPMEVDEKKPEVKVEVKEEEESSSNGTASQSTSPSQPRKKIFKPEELRQALMPTLEALYRQDPESLPFRQPVDPQLLGIPDYFDIVKNPMDLSTIKRKLDTGQYQEPWQYVDDVWLMFNNAWLYNRKTSRVYKFCSKLAEVFEQEIDPVMQSLGYCCGRKYEFSPQTLCCYGKQLCTIPRDAAYYSYQNRYHFCEKCFTEIQGENVTLGDDPSQPQTTISKDQFEKKKNDTLDPEPFVDCKECGRKMHQICVLHYDIIWPSGFVCDNCLKKTGRPRKENKFSAKRLQTTRLGNHLEDRVNKFLRRQNHPEAGEVFVRVVASSDKTVEVKPGMKSRFVDSGEMSESFPYRTKALFAFEEIDGVDVCFFGMHVQEYGSDCPPPNTRRVYISYLDSIHFFRPRCLRTAVYHEILIGYLEYVKKLGYVTGHIWACPPSEGDDYIFHCHPPDQKIPKPKRLQEWYKKMLDKAFAERIIHDYKDIFKQATEDRLTSAKELPYFEGDFWPNVLEESIKELEQEEEERKKEESTAASETTEGSQGDSKNAKKKNNKKTNKNKSSISRANKKKPSMPNVSNDLSQKLYATMEKHKEVFFVIHLHAGPVINTLPPIVDPDPLLSCDLMDGRDAFLTLARDKHWEFSSLRRSKWSTLCMLVELHTQGQDRFVYTCNECKHHVETRWHCTVCEDYDLCINCYNTKSHAHKMVKWGLGLDDEGSSQGEPQSKSPQESRRLSIQRCIQSLVHACQCRNANCSLPSCQKMKRVVQHTKGCKRKTNGGCPVCKQLIALCCYHAKHCQENKCPVPFCLNIKHKLRQQQIQHRLQQAQLMRRRMATMNTRNVPQQSLPSPTSAPPGTPTQQPSTPQTPQPPAQPQPSPVSMSPAGFPSVARTQPPTTVSTGKPTSQVPAPPPPAQPPPAAVEAARQIEREAQQQQHLYRVNINNSMPPGRTGMGTPGSQMAPVSLNVPRPNQVSGPVMPSMPPGQWQQAPLPQQQPMPGLPRPVISMQAQAAVAGPRMPSVQPPRSISPSALQDLLRTLKSPSSPQQQQQVLNILKSNPQLMAAFIKQRTAKYVANQPGMQPQPGLQSQPGMQPQPGMHQQPSLQNLNAMQAGVPRPGVPPQQQAMGGLNPQGQALNIMNPGHNPNMASMNPQYREMLRRQLLQQQQQQQQQQQQQQQQQQGSAGMAGGMAGHGQFQQPQGPGGYPPAMQQQQRMQQHLPLQGSSMGQMAAQMGQLGQMGQPGLGADSTPNIQQALQQRILQQQQMKQQIGSPGQPNPMSPQQHMLSGQPQASHLPGQQIATSLSNQVRSPAPVQSPRPQSQPPHSSPSPRIQPQPSPHHVSPQTGSPHPGLAVTMASSIDQGHLGNPEQSAMLPQLNTPSRSALSSELSLVGDTTGDTLEKFVEGL

(19) United States
(12) Patent Application Publication
(10) Pub. No.: US 2023/0201212 A1
(43) Pub. Date: Jun. 29, 2023

(54) INHIBITORS OF EZH2 AND METHODS OF USE THEREOF

(71) Applicant: Epizyme, Inc., Cambridge, MA (US)

(72) Inventors: Stephen BLAKEMORE, Littleton, MA (US); Scott Richard DAIGLE, Newburyport, MA (US)

(21) Appl. No.: 16/622,391

(22) PCT Filed: Jun. 13, 2018
(86) PCT No.: PCT/US2018/037321

§ 371 Date: Dec. 13, 2019

Related U.S. Application Data
(60) Provisional application No. 62/519,078, filed on Jun. 13, 2017.

(51) Int. Cl. A61K 31/5377; C12Q 1/6886

(52) U.S. Cl. CPC A61K 31/5377; C12Q 1/6886; C12Q 2600/156

(57) ABSTRACT

The disclosure provides a method of treating cancer in a subject in need thereof including administering to the subject a therapeutically-effective amount of an enhancer of a zeste homolog 2 (EZH2) inhibitor. In certain embodiments of this method, the subject has one or more mutations, or exhibits a genetic profile, listed in Tables 1-5, and/or FIGS. 1-3.

### RELATED APPLICATION

This application is a U.S. National Phase application, filed under 35 U.S.C. & 371. of International Application No. PCT/US2018/037321, filed Jun. 13, 2018, which claims priority to, and the benefit of, U.S. Provisional Application No. 62/519,078, filed Jun. 13, 2017, the contents of each of which are incorporated herein by reference in their entireties

### SEQUENCE LISTING

The present application is being filed along with a Sequence Listing in electronic format. The Sequence Listing is provided as a file entitled “EPIZ-083001WO_ST25.txt”, which is 205,913 bytes in size. The information in the electronic format of the sequence listing is incorporated herein by reference in its entirety.

### BACKGROUND

There is a long-felt yet unmet need for effective treatments for certain cancers caused by genetic alterations that result in EZH2-dependent oncogenesis.

### SUMMARY

In some aspects, the disclosure provides a method of treating cancer comprising administering a therapeutically effective amount of an inhibitor of Enhancer to Zeste Homolog 2 (EZH2) to a subject in need thereof, wherein the cancer is characterized by at least one mutation in one or more sequences encoding a gene or gene product listed in Tables 1-5, and/or FIGS. 1-3. In some embodiments, the cancer is characterized by at least one mutation in one or more sequences encoding: EZH2, MYD88, STAT6A, MYC, TP53, and/or HIST1H1E. In some embodiments, the cancer is characterized by a genotype that satisfies Genetic Profile 1 of FIG. 3. In some embodiments, the cancer is characterized by a genotype that satisfies Genetic Profile 2 of Table 5. In some embodiments, the cancer comprises a MYD88 gain-of-function mutation. In some embodiments, the cancer does not comprise an EZH2 gain-of-function mutation. In some embodiments, the at least one mutation decreases the function of a protein encoded by the mutated sequence as compared to the function of the protein encoded by the wild-type sequence. In some embodiments, the at least one mutation is a loss-of-function mutation. In some embodiments, the method further comprises detecting the at least one mutation in the subject. In some embodiments, the detecting comprises subjecting a sample obtained from the subject to a sequence analysis assay. In some embodiments, the analysis assay is a cobas® assay. In some embodiments, the detecting comprises obtaining a sample from the subject. In some embodiments, the sample is a tumor sample or a sample that comprises a cancer cell. In some embodiments, the sample is a sample comprising ctDNA. In some embodiments, the inhibitor of EZH2 is

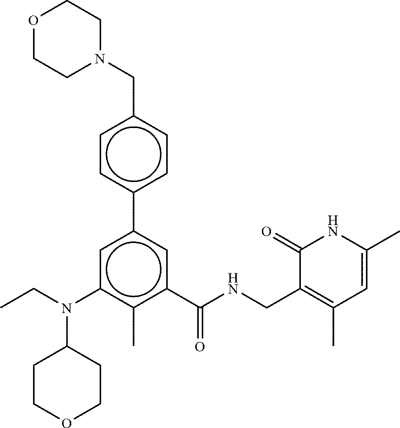

(tazemetostat), or a pharmaceutically-acceptable salt thereof.

In some embodiments, the inhibitor of EZH2 is administered orally. In some embodiments, the inhibitor of EZH2 is formulated as a tablet. In some embodiments, the therapeutically effective amount of the inhibitor of EZH2 is between 100 mg and 3200 mg per day. In some embodiments, the therapeutically effective amount of the inhibitor of EZH2 is 100 mg, 200 mg, 400 mg, 600 mg, 800 mg, 1000 mg, 1200 mg, 1400 mg, 1600 mg or 3200 mg per day. In some embodiments, the therapeutically effective amount is 1600 mg per day. In some embodiments, the cancer expresses a wild type EZH2 protein and does not express a mutant EZH2 protein. In some embodiments, the cancer expresses a mutant EZH2 protein. In some embodiments, the mutant EZH2 protein comprises a substitution of any amino acid other than tyrosine (Y) for tyrosine (Y) at position 641 of SEQ ID NO: 1. In some embodiments, the mutant EZH2 protein comprises a substitution of any amino acid other than alanine (A) for alanine (A) at position 682 of SEQ ID NO: 1. In some embodiments, the mutant EZH2 protein comprises a substitution of any amino acid other than alanine (A) for alanine (A) at position 692 of SEQ ID NO: 1. In some embodiments, the at least one mutation comprises a MYD88 and/or STAT6A mutation, preferably wherein the MYD88 mutation is a gain-of-function mutation, preferably a MYD88 L273 mutation. In some embodiments, the cancer does not have a MYC and/or a HIST1H1E mutation. In some embodiments, the cancer (a) has a MYD88 gain-of-function or a STAT6A loss-of-function mutation, and (b) has neither a MYC nor a HIST1H1E mutation. In some embodiments, the cancer (a) has a MYD88 L273 mutation or a STAT6A loss-of-function mutation, (b) has neither a MYC nor a HIST1H1E mutation, and (c) does not have an EZH2 gain-of-function mutation. In some embodiments, the subject is a human subject. In some embodiments, the subject has cancer. In some embodiments, the cancer is B-cell lymphoma. In some embodiments, the B-cell lymphoma is an activated B-cell (ABC) type. In some embodiments, the B-cell lymphoma is a germinal B-cell (GBC) type. In some embodiments, the cancer is follicular lymphoma.

Some aspects of this disclosure provide methods comprising selecting a subject having cancer for treatment with an EZH2 inhibitor based on the presence of at least one mutation associated with a positive response to such treatment in the subject and/or based on the absence of at least one mutation associated with no response or with a negative response to such treatment in the subject. In some embodiments, the at least one mutation associated with a positive response comprise (a) an EZH2 gain-of-function mutation or a MYD88 gain-of-function mutation; and (b) a STAT6 loss-of-function mutation. In some embodiments, the at least one mutation associated with no response or with a negative response comprise (a) a MYC mutation; (b) a TP53 mutation; and/or (c) a HIST1H1E mutation. In some embodiments, the method comprises detecting the at least one mutation associated with a positive response and/or the at least one mutation associated with no response or a negative response in a sample obtained from the subject. In some embodiments, the method comprises selecting the subject for treatment with the EZH2 inhibitor based on the subject (a) having either an EZH2 or a MYD88 gain-of-function mutation, or a STAT6A loss-of-function mutation; and (b) having neither a MYC mutation, nor a TP53 mutation, nor a HIST1H1E mutation. In some embodiments, the method comprises selecting the subject for treatment with the EZH2 inhibitor based on the subject (a) having a MYD88 L273 mutation or a STAT6A mutation, and (b) having neither a MYC mutation, nor a TP53 mutation, nor a HIST1H1E mutation. In some embodiments, the at least one mutation consists of a single mutation. In some embodiments, the at least one mutation comprises 2 mutations or more. In some embodiments, the at least one mutation comprises 3 mutations or more. In some embodiments, the at least one mutation comprises 4 mutations or more.

Some aspects of this disclosure provide methods comprising selecting a subject having cancer for treatment with an EZH2 inhibitor based on the presence of a mutation profile in the subject that comprises one or more mutations described for a patient exhibiting a complete or partial response or stable disease in any of Tables 1-5 or FIGS. 1-3, or that comprises, encompasses, or matches the complete mutation profile of such a patient, or that comprises, encompasses, or matches Genetic Profile 1 or Genetic Profile 2 of Table 5. .

The methods disclosed herein may have one or more of the following features.

Exemplary sequences associated with the genes and proteins provided herein include, without limitation: MYD88 (e.g., GenBank Accession No. NM_001172567.1, NM_002468.4, NM_001172568.1, NM_001172569.1, and NM_001172566.1), STAT6A (e.g., GenBank Accession No. NM_001178078.1, NM_003153.4, NM_001178079.1, NM_001178080.1, or NM_001178081.1), SOCS1 (e.g., GenBank Accession No. NM_003745.1), MYC (e.g., GenBank Accession No. NM_002467.4), HIST1H1E (e.g., GenBank Accession No. NM_005321.2), ABL1 (e.g., GenBank Accession No. NM_005157), ACVR1 (e.g., GenBank Accession No. NM_001105.4), AKT1 (e.g., GenBank Accession No. NM_001014431.1), AKT2 (e.g., GenBank Accession No. NM_001243027.2), ALK (e.g., GenBank Accession No. NM_004304.4), APC (e.g., GenBank Accession No. NM_000038.5), AR (e.g., GenBank Accession No. NM_000044.3), ARID1A (e.g., GenBank Accession No. NM_006015.4), ARID1B (e.g., GenBank Accession No. NM_020732.3), ASXL1 (e.g., GenBank Accession No. NM_015338.5), ATM (e.g., GenBank Accession No. NM_000051.3), ATRX (e.g., GenBank Accession No. NM_000489.4), AURKA (e.g., GenBank Accession No. NM_003600.3), AXIN2 (e.g., GenBank Accession No. NM_004655.3), BAP1 (e.g., GenBank Accession No. NM_004656.3), BCL2 (e.g., GenBank Accession No. NM_000633.2), BCR (e.g., GenBank Accession No. X02596.1), BLM (e.g., GenBank Accession No. NM_000057.3), BMPR1A (e.g., GenBank Accession No. NM_004329.2), BRAF (e.g., GenBank Accession No. NM_004333.4), BRCA1 (e.g., GenBank Accession No. NM_007294.3), BRCA2 (e.g., GenBank Accession No. NM_000059.3), BRIP1 (e.g., GenBank Accession No. NM_032043.21), BTK (e.g., GenBank Accession No. NM_001287344.1), BUB1B (e.g., GenBank Accession No. NM_001211.5), CALR (e.g., GenBank Accession No. NM_004343.3), CBL (e.g., GenBank Accession No. NM_005188.3), CCND1 (e.g., GenBank Accession No. NM_053056.2), CCNE1 (e.g., GenBank Accession No. NM_001322262.1), CDC73 (e.g., GenBank Accession No. NM_024529.4), CDH1 (Accession No. NM_001317186.1), CDK4 (e.g., GenBank Accession No. NM_000075.3), CDK6 (e.g., GenBank Accession No. NM_001145306.1), CDKN1B (e.g., GenBank Accession No. NM_004064.4), CDKN2A (e.g., GenBank Accession No. NM_001195132.1), CDKN2B (e.g., GenBank Accession No. NM_078487.2), CDKN2C (e.g., GenBank Accession No. NM_078626.2), CEBPA (e.g., GenBank Accession No. NM_001285829.1), CHEK2 (e.g., GenBank Accession No. NM_145862.2), CIC (e.g., GenBank Accession No. NM_015125.4), CREBBP (e.g., GenBank Accession No. NM_001079846.1), CSF1R (e.g., GenBank Accession No. NM_001288705.2), CTNNB1 (e.g., GenBank Accession No. NM_001098209.1), CYLD (e.g., GenBank Accession No. NM_001042355.1), DAXX (Accession No. NM_001141969.1), DDB2 (e.g., GenBank Accession No. NM_001300734.1), DDR2 (e.g., GenBank Accession No. NM_001014796.1), DICER1 (e.g., GenBank Accession No. NM_001291628.1), DNMT3A (e.g., GenBank Accession No. NM_001320893.1), EGFR (e.g., GenBank Accession No. NM_001346900.1), EP300 (e.g., GenBank Accession No. NM_001429.3), ERBB2 (e.g., GenBank Accession No. NM_001289936.1), ERBB3 (e.g., GenBank Accession No. NM_001982.3), ERBB4 (e.g., GenBank Accession No. NM_005235.2), ERCC1 (e.g., GenBank Accession No. NM_001166049.1), ERCC2 (e.g., GenBank Accession No. NM_001130867.1), ERCC3 (e.g., GenBank Accession No. NM_001303418.1), ERCC4 (Accession No. NM_005236.2), ERCC5 (e.g., GenBank Accession No. NM_000123.3), ESR1 (e.g., GenBank Accession No. NM_001291241.1), ETV1 (e.g., GenBank Accession No. NM_001163147.1), ETV5 (Accession No. NM_004454.2), EWSR1 (e.g., GenBank Accession No. NM_001163287.1), EXT1 (e.g., GenBank Accession No. NM_000127.2), EXT2 (Accession No. NM_001178083.1), FANCA (e.g., GenBank Accession No. NM_001286167.1), FANCB (Accession No. NM_001324162.1), FANCC (e.g., GenBank Accession No. NM_001243744.1), FANCD2 (e.g., GenBank Accession No. NM_001319984.1), FANCE (e.g., GenBank Accession No. NM_021922.2), FANCF (e.g., GenBank Accession No NM_022725.3.), FANCG (e.g., GenBank Accession No. NM_004629.1), FANCI (e.g., GenBank Accession No. NM_018193.2), FANCL (Accession No. NM_001114636.1), FANCM (e.g., GenBank Accession No. NM_001308133.1), FBXW7 (e.g., GenBank Accession No. NM_018315.4), FGFR1 (Accession No.) NM_001174065.1, FGFR2 (e.g., GenBank Accession No. NM_000141.4), FGFR3 (e.g., GenBank Accession No. NM_001163213.1), FGFR4 (e.g., GenBank Accession No. NM_213647.2), FH (e.g., GenBank Accession No. NM_000143.3), FLCN (e.g., GenBank Accession No. NM_144606.5), FLT3 (e.g., GenBank Accession No. NM_004119.2), FLT4 (e.g., GenBank Accession No. NM_002020.4), FOXL2 (e.g., GenBank Accession No. NM_023067.3), GATA1 (e.g., GenBank No. NM_002049.3), GATA2 (e.g., GenBank Accession No. NM_001145662.1), GNA11 (e.g., GenBank Accession No. NM_002067.4), GNAQ (e.g., GenBank Accession No. NM_002072.4), GNAS (e.g., GenBank Accession No. NM_080425.3), GPC3 (e.g., GenBank Accession No. NM_001164619.1), H3F3A (e.g., GenBank Accession No. NM_002107.4), H3F3B (e.g., GenBank Accession No. NM_005324.4), HNF1A (e.g., GenBank Accession No. NM_000545.6), HRAS (e.g., GenBank Accession No. NM_001130442.2), IDH1 (e.g., GenBank Accession No. NM_001282387.1), IDH2 (e.g., GenBankAccession No. NM_001290114.1), IGF1R (e.g., GenBank Accession No. NM_001291858.1), IGF2R (e.g., GenBank Accession No. NM_000876.3), IKZF1 (e.g., GenBank Accession No. NM_001291847.1), JAK1 (e.g., GenBank Accession No. NM_001321857.1), JAK2 (e.g., GenBank Accession No. NM_001322195.1), JAK3 (e.g., GenBank Accession No. NM_000215.3), KDR (e.g., GenBank Accession No. NM_002253.2), KIT (e.g., GenBank Accession No. NM_001093772.1), KRAS (e.g., GenBank Accession No. NM_033360.3), MAML1 (e.g., GenBank Accession No. NM_014757.4), MAP2K1 (e.g., GenBank Accession No. NM_002755.3), MAP2K4 (e.g., GenBank Accession No. NM_001281435.1), MDM2 (e.g., GenBank Accession No. NM_001145337.2), MDM4 (e.g., GenBank Accession No. NM_001278519.1), MED12 (e.g., GenBank Accession No. NM_005120.2), MEN1 (e.g., GenBank Accession No. NM_130804.2), MET (e.g., GenBank Accession No NM_000245.3), MLH1 (e.g., GenBank Accession No. NM_000249.3), MLL (e.g., GenBank Accession No. AF232001.1), MPL (e.g., GenBank Accession No. NM_005373.2), MSH2 (e.g., GenBank Accession No. NM_000251.2), MSH6 (e.g., GenBank Accession No. NM_000179.2), MTOR (Accession No. NM_004958.3), MUTYH (e.g., GenBank Accession No. NM_001048171.1), MYC (e.g., GenBank Accession No. NM_002467.4), MYCL1 (e.g., GenBank Accession No NM _001033081.2), MYCN (e.g., GenBank Accession No. NM_001293231.1), NBN (e.g., GenBank Accession No. NM_001024688.2), NCOA3 (e.g., GenBank Accession No. NM_001174087.1), NF1 (e.g., GenBank Accession No. NM_001042492.2), NF2 (e.g., GenBank Accession No. NM_181831.2), NKX2-1(e.g., GenBank Accession No. NM_001079668.2), NOTCH1 (e.g., GenBank Accession No. NM_017617.4), NOTCH2 (e.g., GenBank Accession No NM_001200001.1), NOTCH3 (e.g., GenBank Accession No. NM_000435.2), NOTCH4 (Accession No. NR_134950.1), NPM1 (e.g., GenBank Accession No. NM_002520.6), NRAS (Accession No. NM_002524.4), NTRK1 (e.g., GenBank Accession No. NM_001007792.1), PALB2 (e.g., GenBank Accession No. NM_024675.3), PAX5 (e.g., GenBank Accession No. NM_001280552.1), PBRM1 (e.g., GenBank Accession No. NM_181042.4), PDGFRA (e.g., GenBank Accession No. NM_006206.4), PHOX2B (e.g., GenBank Accession No. NM_003924.3), PIK3CA (e.g., GenBank Accession No. NM_006218.3), PIK3R1 (Accession No. NM_001242466.1), PMS1 (e.g., GenBank Accession No. NM_001321051.1), PMS2 (e.g., GenBank Accession No. NM_000535.6), POLD1 (e.g., GenBank Accession No. NM_001308632.1), POLE (e.g., GenBank Accession No. NM_006231.3), POLH (e.g., GenBank Accession No. NM_001291970.1), POT1 (e.g., GenBank Accession No. NM_001042594.1), PRKAR1A (e.g., GenBank Accession No. NM_001278433.1), PRSS1 (e.g., GenBank Accession No. NM_002769.4), PTCH1 (e.g., GenBank Accession No. NM_000264.3), PTEN (e.g., GenBank Accession No. NM_000314.6), PTPN11 (e.g., GenBank Accession No. NM_001330437.1), RAD51C (e.g., GenBank Accession No. NR_103873.1), RAF1 (e.g., GenBank Accession No. NM_002880.3), RB1 (e.g., GenBank Accession No. NM_000321.2), RECQL4 (e.g., GenBank Accession No. NM_004260.3), RET (e.g., GenBank Accession No.), RNF43(e.g., GenBank Accession No. NM_017763.5), ROS1 (e.g., GenBank Accession No. NM_002944.2), RUNX1 (e.g., GenBank Accession No. NM_001122607.1), SBDS (e.g., GenBank Accession No. NM_016038.2), SDHAF2 (e.g., GenBank Accession No. NM_017841.2), SDHB (e.g., GenBank Accession No.), SDHC (e.g., GenBank Accession No.), SDHD (e.g., GenBank Accession No. NM_001276503.1), SF3B1 (e.g., GenBank Accession No. NM_001308824.1), SMAD2 (e.g., GenBank Accession No. NM_001135937.2), SMAD3 (e.g., GenBank Accession No. NM_001145104.1), SMAD4 (e.g., GenBank Accession No. NM_005359.5), SMARCB1 (e.g., GenBank Accession No. NM_001007468.2), SMO (e.g., GenBank Accession No. NM_005631.4), SRC (e.g., GenBank Accession No. NM_005417.4), STAG2 (e.g., GenBank Accession No. NM_001282418.1), STK11 (e.g., GenBank Accession No. NM_000455.4), SUFU (e.g., GenBank Accession No. NM_001178133.1), TERT (e.g., GenBank Accession No. NM_001193376.1), TET2 (e.g., GenBank Accession No. NM_017628.4), TGFBR2 (e.g., GenBank Accession No. NM_001024847.2), TNFAIP3 (e.g., GenBank Accession No. NM_001270508.1), TOP1 (e.g., GenBank Accession No. NM_003286.3), TP53 (e.g., GenBank Accession No. NM_000546.5), TSC1 (e.g., GenBank Accession No. NM_001162427.1), TSC2 (e.g., GenBank Accession No. NM_001318832.1), TSHR (e.g., GenBank Accession No. NM_000369.2), VHL (e.g., GenBank Accession No. NM_000551.3), WAS (e.g., GenBank Accession No. NM_000377.2), WRN (e.g., GenBank Accession No. NM_000553.4), WT1 (e.g., GenBank Accession No. NM_000378.4), XPA (e.g., GenBank Accession No. NM_000380.3), XPC (e.g., GenBank Accession No. NM_004628.4), and/or XRCC1 (e.g., GenBank Accession No. NM_006297.2). It will be understood that the sequences provided above and elsewhere herein are exemplary, and serve to illustrate sequences suitable for some embodiments of the present disclosure. It will also be understood that, in some embodiments, the sequence encoding the gene product referred to herein is a genomic DNA sequence. The skilled artisan will be aware of additional suitable sequences beyond the exemplary, non-limiting sequences provided above, for each gene or gene product (e.g., transcript, mRNA, or protein) referred to herein, or will be able to ascertain such suitable sequences without more than routine effort based on the present disclosure and the knowledge in the art.

In some embodiments, the at least one mutation decreases the function of a protein encoded by the mutated sequence as compared to the function of the protein encoded by the wild-type sequence. In some embodiments, the at least one mutation is a loss-of-function mutation.

In some embodiments, the method further comprises detecting the at least one mutation in the subject.

In some embodiments, the detecting comprises subjecting a sample obtained from the subject to a sequence analysis assay.

In some embodiments, the inhibitor of EZH2 is

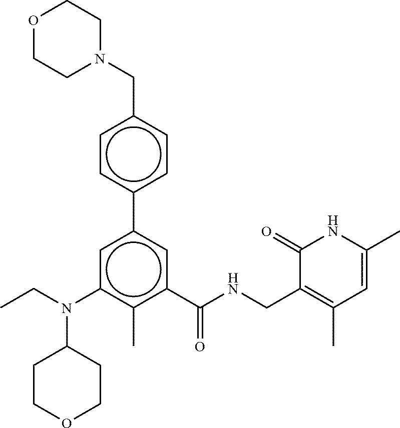

(tazemetostat), or a pharmaceutically-acceptable salt thereof.

In some embodiments, the inhibitor of EZH2 is administered orally.

In some embodiments, the inhibitor of EZH2 is formulated as a tablet.

In some embodiments, the therapeutically effective amount of the inhibitor of EZH2 is between 100 mg and 3200 mg per day. -In some embodiments, the therapeutically effective amount of the inhibitor of EZH2 is 100 mg, 200 mg, 400 mg, 600 mg, 800 mg, 1000 mg, 1200 mg, 1400 mg, 1600 mg or 3200 mg per day. In some embodiments, the therapeutically effective amount is 1600 mg per day. In some embodiments, the therapeutically effective amount of the inhibitor of is administered at 800 mg twice per day (BID).

In some embodiments, the subject or cancer expresses a wild type EZH2 protein and/or does not express a mutant EZH2 protein.

In some embodiments, the subject or cancer expresses a mutant EZH2 protein. In some embodiments, the mutant EZH2 protein comprises a substitution of any amino acid other than tyrosine (Y) for tyrosine (Y) at position 641 of SEQ ID NO: 1. In some embodiments, the mutant EZH2 protein comprises a substitution of any amino acid other than alanine (A) for alanine (A) at position 682 of SEQ ID NO: 1. In some embodiments, the mutant EZH2 protein comprises a substitution of any amino acid other than alanine (A) for alanine (A) at position 692 of SEQ ID NO: 1.

In some embodiments, the at least one mutation consists of a single mutation. In some embodiments, the at least one mutation comprises 2 mutations or more. In some embodiments, the at least one mutation comprises 3 mutations or more. In some embodiments, the at least one mutation comprises 4 mutations or more. In some embodiments, the at least one mutation comprises 5 mutations or more.

In some embodiments, the at least one mutation comprises 2 mutations, 3 mutations, 4 mutations, 5 mutations, 6 mutations, 7 mutations, 8 mutations, 9 mutations, 10 mutations, 11 mutations, 12 mutations, 13 mutations, 14 mutations, 15 mutations, 16 mutations, 17 mutations, 18 mutations, 19 mutations, or 20 mutations.

In some embodiments, the at least one mutation comprises at least one positive mutation (e.g., with or without a negative mutation). In some embodiments, the at least one mutation comprises at least one negative mutation (e.g., with or without a positive mutation). In some embodiments, the at least one mutation comprises both positive and negative mutations. The term “positive mutation”, as used herein, refers to a mutation that sensitizes a subject, a cancer, or malignant cell or population of cells, to EZH2 treatment, or, in some embodiments, that renders a subject, cancer, or malignant cell or population of cells, more sensitive to EZH2 treatment. The term “negative mutation”, as used herein, refers to a mutation that desensitizes a subject, a cancer, or malignant cell or population of cells, to EZH2 treatment, or, in some embodiments, that renders a subject, cancer, or malignant cell or population of cells, less sensitive to EZH2 treatment. In some embodiments, the disclosure provides a method of identifying molecular variants in tumor samples harvested from NHL patients treated with a compound of the disclosure. In some embodiments, the disclosure provides a method of identifying molecular variants in cell free circulating tumor DNA (ctDNA) harvested from NHL patients treated with a compound of the disclosure.

In some embodiments, the molecular variants identified therein may correlate with clinical response, minimal residual disease or emergence of resistance.

The summary above is meant to illustrate, in a non-limiting manner, some of the embodiments, advantages, features, and uses of the technology disclosed herein. Other embodiments, advantages, features, and uses of the technology disclosed herein will be apparent from the Detailed Description, the Drawings, the Examples, and the Claims.

### BRIEF DESCRIPTION OF THE DRAWINGS

The above and further features will be more clearly appreciated from the following detailed description when taken in conjunction with the accompanying drawings.

FIG. 1 shows an overview of the analytical and statistical methods used in this study.

FIGS. 2A-2D show somatic mutations, amplifications, and translocations detected in 92 NHL patients by best overall response. FIG. 2A shows all 92 NHL patients assessed. FIG. 2B shows NHL patients classified as responders (complete response (CR) or partial response (PR)). FIG. 2C shows FL patients that were classified as non-responders. FIG. 2D shows DLBCL patients that were classified as non-responders. NHL: Non-Hodgkin Lymphoma; CR: complete response; PR: partial response; SD: stable disease; PD: progressive disease; UK: unknown response. Patients with CR and PR were classified as responders. Patients with SD, PD, and UK were classified as non-responders.

FIG. 3 shows variants identified as significantly associated with tazemetostat response using gene level analysis of archive tumor tissue distributed by response-favoring mutant and response favoring wild-type gene (N=92).

FIG. 4 is a structure model of partial EZH2 protein based on the A chain of nuclear receptor binding SET domain protein 1 (NSD1). This model corresponds to amino acid residues 533-732 of EZH2 sequence of SEQ ID NO: 1.

### DETAILED DESCRIPTION

Tazemetostat demonstrates clinical activity as a monotherapy in patients with relapsed or refractory DLBCL (both GCB and non-GCB), follicular lymphoma (FL) and marginal zone lymphomas (MZL). Objective responses in tumors with either wild-type or mutation in EZH2 are durable as patients are ongoing at 7+ to 21+ months. Safety profile as monotherapy continues to be acceptable and favorable for combination development. Recommended phase II dose (RP2D) of 800 mg BID supported by safety, efficacy, PK and PD.

Baseline somatic mutation profiling revealed associations between objective response to tazemetostat and genetic alterations, e.g., mutations in genomic sequences encoding MYD88, STAT6A, SOCS1, MYC, TP53, HIST1H1E, and histone acetyltransferases, such as, for example CREBBP and EP300.

### EZH2

EZH2 is a histone methyltransferase that is the catalytic subunit of the PRC2 complex which catalyzes the mono- through tri-methylation of lysine 27 on histone H3 (H3-K27).

Point mutations of the EZH2 gene at a single amino acid residue (e.g., Tyr641, herein referred to as Y641) of EZH2 have been reported to be linked to subsets of human B-cell lymphoma. Morin et al. (2010) Nat Genet 42(2): 181-5. In particular, Morin et al. reported that somatic mutations of tyrosine 641 (Y641F, Y641H, Y641N, and Y641S) of EZH2 were associated with follicular lymphoma (FL) and the germinal center B cell-like (GCB) subtype of diffuse large B-cell lymphoma (DLBCL). The mutant allele is always found associated with a wild-type allele (heterozygous) in disease cells, and the mutations were reported to ablate the enzymatic activity of the PRC2 complex for methylating an unmodified peptide substrate.

The mutant EZH2 refers to a mutant EZH2 polypeptide or a nucleic acid sequence encoding a mutant EZH2 polypeptide. Preferably the mutant EZH2 comprises one or more mutations in its substrate pocket domain as defined in SEQ ID NO: 6. For example, the mutation may be a substitution, a point mutation, a nonsense mutation, a missense mutation, a deletion, or an insertion. Exemplary substitution amino acid mutation includes a substitution at amino acid position 677, 687, 674, 685, or 641 of SEQ ID NO: 1, such as, but is not limited to a substitution of glycine (G) for the wild type residue alanine (A) at amino acid position 677 of SEQ ID NO: 1 (A677G); a substitution of valine (V) for the wild type residue alanine (A) at amino acid position 687 of SEQ ID NO: 1 (A687V); a substitution of methionine (M) for the wild type residue valine (V) at amino acid position 674 of SEQ ID NO: 1 (V674M); a substitution of histidine (H) for the wild type residue arginine (R) at amino acid position 685 of SEQ ID NO: 1 (R685H); a substitution of cysteine (C) for the wild type residue arginine (R) at amino acid position 685 of SEQ ID NO: 1 (R685C); a substitution of phenylalanine (F) for the wild type residue tyrosine (Y) at amino acid position 641 of SEQ ID NO: 1 (Y641F); a substitution of histidine (H) for the wild type residue tyrosine (Y) at amino acid position 641 of SEQ ID NO: 1 (Y641H); a substitution of asparagine (N) for the wild type residue tyrosine (Y) at amino acid position 641 of SEQ ID NO: 1 (Y641N); a substitution of serine (S) for the wild type residue tyrosine (Y) at amino acid position 641 of SEQ ID NO: 1 (Y641S); or a substitution of cysteine (C) for the wild type residue tyrosine (Y) at amino acid position 641 of SEQ ID NO: 1 (Y641C).

The mutation may also include a substitution of serine (S) for the wild type residue asparagine (N) at amino acid position 322 of SEQ ID NO: 3 (N322S), a substitution of glutamine (Q) for the wild type residue arginine (R) at amino acid position 288 of SEQ ID NO: 3 (R288Q), a substitution of isoleucine (I) for the wild type residue threonine (T) at amino acid position 573 of SEQ ID NO: 3 (T573I), a substitution of glutamic acid (E) for the wild type residue aspartic acid (D) at amino acid position 664 of SEQ ID NO: 3 (D664E), a substitution of glutamine (Q) for the wild type residue arginine (R) at amino acid position 458 of SEQ ID NO: 5 (R458Q), a substitution of lysine (K) for the wild type residue glutamic acid (E) at amino acid position 249 of SEQ ID NO: 3 (E249K), a substitution of cysteine (C) for the wild type residue arginine (R) at amino acid position 684 of SEQ ID NO: 3 (R684C), a substitution of histidine (H) for the wild type residue arginine (R) at amino acid position 628 of SEQ ID NO: 21 (R628H), a substitution of histidine (H) for the wild type residue glutamine (Q) at amino acid position 501 of SEQ ID NO: 5 (Q501H), a substitution of asparagine (N) for the wild type residue aspartic acid (D) at amino acid position 192 of SEQ ID NO: 3 (D192N), a substitution of valine (V) for the wild type residue aspartic acid (D) at amino acid position 664 of SEQ ID NO: 3 (D664V), a substitution of leucine (L) for the wild type residue valine (V) at amino acid position 704 of SEQ ID NO: 3 (V704L), a substitution of serine (S) for the wild type residue proline (P) at amino acid position 132 of SEQ ID NO: 3 (P132S), a substitution of lysine (K) for the wild type residue glutamic acid (E) at amino acid position 669 of SEQ ID NO: 21 (E669K), a substitution of threonine (T) for the wild type residue alanine (A) at amino acid position 255 of SEQ ID NO: 3 (A255T), a substitution of valine (V) for the wild type residue glutamic acid (E) at amino acid position 726 of SEQ ID NO: 3 (E726V), a substitution of tyrosine (Y) for the wild type residue cysteine (C) at amino acid position 571 of SEQ ID NO: 3 (C571Y), a substitution of cysteine (C) for the wild type residue phenylalanine (F) at amino acid position 145 of SEQ ID NO: 3 (F145C), a substitution of threonine (T) for the wild type residue asparagine (N) at amino acid position 693 of SEQ ID NO: 3 (N693T), a substitution of serine (S) for the wild type residue phenylalanine (F) at amino acid position 145 of SEQ ID NO: 3 (F145S), a substitution of histidine (H) for the wild type residue glutamine (Q) at amino acid position 109 of SEQ ID NO: 21 (Q109H), a substitution of cysteine (C) for the wild type residue phenylalanine (F) at amino acid position 622 of SEQ ID NO: 21 (F622C), a substitution of arginine (R) for the wild type residue glycine (G) at amino acid position 135 of SEQ ID NO: 3 (G135R), a substitution of glutamine (Q) for the wild type residue arginine (R) at amino acid position 168 of SEQ ID NO: 5 (R168Q), a substitution of arginine (R) for the wild type residue glycine (G) at amino acid position 159 of SEQ ID NO: 3 (G159R), a substitution of cysteine (C) for the wild type residue arginine (R) at amino acid position 310 of SEQ ID NO: 5 (R310C), a substitution of histidine (H) for the wild type residue arginine (R) at amino acid position 561 of SEQ ID NO: 3 (R561H), a substitution of histidine (H) for the wild type residue arginine (R) at amino acid position 634 of SEQ ID NO: 21 (R634H), a substitution of arginine (R) for the wild type residue glycine (G) at amino acid position 660 of SEQ ID NO: 3 (G660R), a substitution of cysteine (C) for the wild type residue tyrosine (Y) at amino acid position 181 of SEQ ID NO: 3 (Y181C), a substitution of arginine (R) for the wild type residue histidine (H) at amino acid position 297 of SEQ ID NO: 3 (H297R), a substitution of serine (S) for the wild type residue cysteine (C) at amino acid position 612 of SEQ ID NO: 21 (C612S), a substitution of tyrosine (Y) for the wild type residue histidine (H) at amino acid position 694 of SEQ ID NO: 3 (H694Y), a substitution of alanine (A) for the wild type residue aspartic acid (D) at amino acid position 664 of SEQ ID NO: 3 (D664A), a substitution of threonine (T) for the wild type residue isoleucine (I) at amino acid position 150 of SEQ ID NO: 3 (I150T), a substitution of arginine (R) for the wild type residue isoleucine (I) at amino acid position 264 of SEQ ID NO: 3 (I264R), a substitution of leucine (L) for the wild type residue proline (P) at amino acid position 636 of SEQ ID NO: 3 (P636L), a substitution of threonine (T) for the wild type residue isoleucine (I) at amino acid position 713 of SEQ ID NO: 3 (I713T), a substitution of proline (P) for the wild type residue glutamine (Q) at amino acid position 501 of SEQ ID NO: 5 (Q501P), a substitution of glutamine (Q) for the wild type residue lysine (K) at amino acid position 243 of SEQ ID NO: 3 (K243Q), a substitution of aspartic acid (D) for the wild type residue glutamic acid (E) at amino acid position 130 of SEQ ID NO: 5 (E130D), a substitution of glycine (G) for the wild type residue arginine (R) at amino acid position 509 of SEQ ID NO: 3 (R509G), a substitution of histidine (H) for the wild type residue arginine (R) at amino acid position 566 of SEQ ID NO: 3 (R566H), a substitution of histidine (H) for the wild type residue aspartic acid (D) at amino acid position 677 of SEQ ID NO: 3 (D677H), a substitution of asparagine (N) for the wild type residue lysine (K) at amino acid position 466 of SEQ ID NO: 5 (K466N), a substitution of histidine (H) for the wild type residue arginine (R) at amino acid position 78 of SEQ ID NO: 3 (R78H), a substitution of methionine (M) for the wild type residue lysine (K) at amino acid position 1 of SEQ ID NO: 6 (K6M), a substitution of leucine (L) for the wild type residue serine (S) at amino acid position 538 of SEQ ID NO: 3 (S538L), a substitution of glutamine (Q) for the wild type residue leucine (L) at amino acid position 149 of SEQ ID NO: 3 (L149Q), a substitution of valine (V) for the wild type residue leucine (L) at amino acid position 252 of SEQ ID NO: 3 (L252V), a substitution of valine (V) for the wild type residue leucine (L) at amino acid position 674 of SEQ ID NO: 3 (L674V), a substitution of valine (V) for the wild type residue alanine (A) at amino acid position 656 of SEQ ID NO: 3 (A656V), a substitution of aspartic acid (D) for the wild type residue alanine (A) at amino acid position 731 of SEQ ID NO: 3 (Y731D), a substitution of threonine (T) for the wild type residue alanine (A) at amino acid position 345 of SEQ ID NO: 3 (A345T), a substitution of aspartic acid (D) for the wild type residue alanine (A) at amino acid position 244 of SEQ ID NO: 3 (Y244D), a substitution of tryptophan (W) for the wild type residue cysteine (C) at amino acid position 576 of SEQ ID NO: 3 (C576W), a substitution of lysine (K) for the wild type residue asparagine (N) at amino acid position 640 of SEQ ID NO: 3 (N640K), a substitution of lysine (K) for the wild type residue asparagine (N) at amino acid position 675 of SEQ ID NO: 3 (N675K), a substitution of tyrosine (Y) for the wild type residue aspartic acid (D) at amino acid position 579 of SEQ ID NO: 21 (D579Y), a substitution of isoleucine (I) for the wild type residue asparagine (N) at amino acid position 693 of SEQ ID NO: 3 (N693I), and a substitution of lysine (K) for the wild type residue asparagine (N) at amino acid position 693 of SEQ ID NO: 3 (N693K).

The mutation may be a frameshift at amino acid position 730, 391, 461, 441, 235, 254, 564, 662, 715, 405, 685, 64, 73, 656, 718, 374, 592, 505, 730, or 363 of SEQ ID NO: 3, 5 or 21 or the corresponding nucleotide position of the nucleic acid sequence encoding SEQ ID NO: 3, 5, or 21. The mutation of the EZH2 may also be an insertion of a glutamic acid (E) between amino acid positions 148 and 149 of SEQ ID NO: 3, 5 or 21. Another example of EZH2 mutation is a deletion of glutamic acid (E) and leucine (L) at amino acid positions 148 and 149 of SEQ ID NO: 3, 5 or 21. The mutant EZH2 may further comprise a nonsense mutation at amino acid position 733, 25, 317, 62, 553, 328, 58, 207, 123, 63, 137, or 60 of SEQ ID NO: 3, 5 or 21.

Human EZH2 nucleic acids and polypeptides have previously been described. See, e.g., Chen et al. (1996) Genomics 38:30-7 [746 amino acids]; Swiss-Prot Accession No. Q15910 [746 amino acids]; GenBank Accession Nos. NM_004456 and NP_004447 (isoform a [751 amino acids]); and GenBank Accession Nos. NM_152998 and NP_694543 (isoform b [707 amino acids]), each of which is incorporated herein by reference in its entirety.

Amino acid sequence of human EZH2 (Swiss-Prot
Accession No. Q15910) (SEQ ID NO: 1)
MGQTGKKSEKGPVCWRKRVKSEYMRLRQLKRFRRADEVKSMFSSNRQKIL
ERTEILNQEWKQRRIQPVHILTSVSSLRGTRECSVTSDLDFPTQVIPLKT
LNAVASVPIMYSWSPLQQNFMVEDETVLHNIPYMGDEVLDQDGTFIEELI
KNYDGKVHGDRECGFINDEIFVELVNALGQYNDDDDDDDGDDPEEREEKQ
KDLEDHRDDKESRPPRKFPSDKIFEAISSMFPDKGTAEELKEKYKELTEQ
QLPGALPPECTPNIDGPNAKSVQREQSLHSFHTLFCRRCFKYDCFLHPFH
ATPNTYKRKNTETALDNKPCGPQCYQHLEGAKEFAAALTAERIKTPPKRP
GGRRRGRLPNNSSRPSTPTINVLESKDTDSDREAGTETGGENNDKEEEEK
KDETSSSSEANSRCQTPIKMKPNIEPPENVEWSGAEASMFRVLIGTYYDN
FCAIARLIGTKTCRQVYEFRVKESSIIAPAPAEDVDTPPRKKKRKHRLWA
AHCRKIQLKKDGSSNHVYNYQPCDHPRQPCDSSCPCVIAQNFCEKFCQCS
SECQNRFPGCRCKAQCNTKQCPCYLAVRECDPDLCLTCGAADHWDSKNVS
CKNCSIQRGSKKHLLLAPSDVAGWGIFIKDPVQKNEFISEYCGEIISQDE
ADRRGKVYDKYMCSFLFNLNNDFVVDATRKGNKIRFANHSVNPNCYAKVM
MVNGDHRIGIFAKRAIQTGEELFFDYRYSQADALKYVGIEREMEIP

mRNA sequence of human EZH2, transcript variant 1
(GenBank Accession No. NM_004456) (SEQ ID NO: 2)
ggcggcgcttgattgggctgggggggccaaataaaagcgatggcgattgg
gctgccgcgtttggcgctcggtccggtcgcgtccgacacccggtgggact
cagaaggcagtggagccccggcggcggcggcggcggcgcgcgggggcgac
gcgcgggaacaacgcgagtcggcgcgcgggacgaagaataatcatgggcc
agactgggaagaaatctgagaagggaccagtttgttggcggaagcgtgta
aaatcagagtacatgcgactgagacagctcaagaggttcagacgagctga
tgaagtaaagagtatgtttagttccaatcgtcagaaaattttggaaagaa
cggaaatcttaaaccaagaatggaaacagcgaaggatacagcctgtgcac
atcctgacttctgtgagctcattgcgcgggactagggagtgttcggtgac
cagtgacttggattttccaacacaagtcatcccattaaagactctgaatg
cagttgcttcagtacccataatgtattcttggtctcccctacagcagaat
tttatggtggaagatgaaactgttttacataacattccttatatgggaga
tgaagttttagatcaggatggtactttcattgaagaactaataaaaaatt
atgatgggaaagtacacggggatagagaatgtgggtttataaatgatgaa
atttttgtggagttggtgaatgcccttggtcaatataatgatgatgacga
tgatgatgatggagacgatcctgaagaaagagaagaaaagcagaaagatc
tggaggatcaccgagatgataaagaaagccgcccacctcggaaatttcct
tctgataaaatttttgaagccatttcctcaatgtttccagataagggcac
agcagaagaactaaaggaaaaatataaagaactcaccgaacagcagctcc
caggcgcacttcctcctgaatgtacccccaacatagatggaccaaatgct
aaatctgttcagagagagcaaagcttacactcctttcatacgcttttctg
taggcgatgttttaaatatgactgcttcctacatcgtaagtgcaattatt
cttttcatgcaacacccaacacttataagcggaagaacacagaaacagct
ctaacaacaaaccttgtggaccacagtgttaccagcatttggagggagca
aaggagtttgctgctgctctcaccgctgagcggataaagaccccaccaaa
acgtccaggaggccgcagaagaggacggcttcccaataacagtagcaggc
ccagcacccccaccattaatgtgctggaatcaaaggatacagacagtgat
agggaagcagggactgaaacggggggagagaacaatgataaagaagaaga
agagaagaaagatgaaacttcgagctcctctgaagcaaattctcggtgtc
aaacaccaataaagatgaagccaaatattgaacctcctgagaatgtggag
tggagtggtgctgaagcctcaatgtttagagtcctcattggcacttacta
tgacaatttctgtgccattgctaggttaattgggaccaaaacatgtagac
aggtgtatgagtttagagtcaaagaatctagcatcatagctccagctccc
gctgaggatgtggatactcctccaaggaaaaagaagaggaaacaccggtt
gtgggctgcacactgcagaaagatacagctgaaaaaggacggctcctcta
accatgtttacaactatcaaccctgtgatcatccacggcagccttgtgac
agttcgtgcccttgtgtgatagcacaaaatttttgtgaaaagttttgtca
atgtagttcagagtgtcaaaaccgctttccgggatgccgctgcaaagcac
agtgcaacaccaagcagtgcccgtgctacctggctgtccgagagtgtgac
cctgacctctgtcttacttgtggagccgctgaccattgggacagtaaaaa
tgtgtcctgcaagaactgcagtattcagcggggctccaaaaagcatctat
tgctggcaccatctgacgtggcaggctgggggatttttatcaaagatcct
gtgcagaaaaatgaattcatctgagattatttctcaagatgaagctgaca
gaagagggaaagtgtatgataaatacatgtgcagctttctgttcaacttg
aacaatgattttgtggtggatgcaacccgcaagggtaacaaaattcgttt
tgcaaatcattcggtaaatccaaactgctatgcaaaagttatgatggtta
acggtgatcacaggataggtatttttgccaagagagccatccagactggc
gaagagctgttttttgattacagatacagccaggctgatgccctgaagta
tgtcggcatcgaaagagaaatggaaatcccttgacatctgctacctcctc
ccccctcctctgaaacagctgccttagcttcaggaacctcgagtactgtg
ggcaatttagaaaaagaacatgcagtttgaaattctgaatttgcaaagta
ctgtaagaataatttatagtaatgagtttaaaaatcaactttttattgcc
ttctcaccagctgcaaagtgttttgtaccagtgaatttttgcaataatgc
agtatggtacatttttcaactttgaataaagaatacttgaacttgtcctt
gttgaatc

Full amino acid of EZH2, isoform a (GenBank
Accession No. NP 004447) (SEQ ID NO: 3)
MGQTGKKSEKGPVCWRKRVKSEYMRLRQLKRFRRADEVKSMFSSNRQKIL
ERTEILNQEWKQRRIQPVHILTSVSSLRGTRECSVTSDLDFPTQVIPLKT
LNAVASVPIMYSWSPLQQNFMVEDETVLHNIPYMGDEVLDQDGTFIEELI
KNYDGKVHGDRECGFINDEIFVELVNALGQYNDDDDDDDGDDPEEREEKQ
KDLEDHRDDKESRPPRKFPSDKIFEAISSMFPDKGTAEELKEKYKELTEQ
QLPGALPPECTPNIDGPNAKSVQREQSLHSFHTLFCRRCFKYDCFLHRKC
NYSFHATPNTYKRKNTETALDNKPCGPQCYQHLEGAKEFAAALTAERIKT
PPKRPGGRRRGRLPNNSSRPSTPTINVLESKDTDSDREAGTETGGENNDK
EEEEKKDETSSSSEANSRCQTPIKMKPNIEPPENVEWSGAEASMFRVLIG
TYYDNFCAIARLIGTKTCRQVYEFRVKESSIIAPAPAEDVDTPPRKKKRK
HRLWAAHCRKIQLKKDGSSNHVYNYQPCDHPRQPCDSSCPCVIAQNFCEK
FCQCSSECQNRFPGCRCKAQCNTKQCPCYLAVRECDPDLCLTCGAADHWD
SKNVSCKNCSIQRGSKKHLLLAPSDVAGWGIFIKDPVQKNEFISEYCGEI
ISQDEADRRGKVYDKYMCSFLFNLNNDFVVDATRKGNKIRFANHSVNPNC
YAKVMMVNGDHRIGIFAKRAIQTGEELFFDYRYSQADALKYVGIEREMEI
P

mRNA sequence of human EZH2, transcript variant 2
(GenBank Accession No. NM_152998) (SEQ ID NO: 4)
ggcggcgcttgattgggctgggggggccaaataaaagcgatggcgattgg
gctgccgcgtttggcgctcggtccggtcgcgtccgacacccggtgggact
cagaaggcagtggagccccggcggcggcggcggcggcgcgcgggggcgac
gcgcgggaacaacgcgagtcggcgcgcgggacgaagaataatcatgggcc
agactgggaagaaatctgagaagggaccagtttgttggcggaagcgtgta
aaatcagagtacatgcgactgagacagctcaagaggttcagacgagctga
tgaagtaaagagtatgtttagttccaatcgtcagaaaattttggaaagaa
cggaaatcttaaaccaagaatggaaacagcgaaggatacagcctgtgcac
atcctgacttctgtgagctcattgcgcgggactagggaggtggaagatga
aactgttttacataacattccttatatgggagatgaagttttagatcagg
atggtactttcattgaagaactaataaaaaattatgatgggaaagtacac
ggggatagagaatgtgggtttataaatgatgaaatttttgtggagttggt
gaatgcccttggtcaatataatgatgatgacgatgatgatgatggagacg
atcctgaagaaagagaagaaaagcagaaagatctggaggatcaccgagat
gataaagaaagccgcccacctcggaaatttccttctgataaaatttttga
agccatttcctcaatgtttccagataagggcacagcagaagaactaaagg
aaaaatataaagaactcaccgaacagcagctcccaggcgcacttcctcct
gaatgtacccccaacatagatggaccaaatgctaaatctgttcagagaga
gcaaagcttacactcctttcatacgcttttctgtaggcgatgttttaaat
atgactgcttcctacatccttttcatgcaacacccaacacttataagcgg
aagaacacagaaacagctctagacaacaaaccttgtggaccacagtgtta
ccagcatttggagggagcaaaggagtttgctgctgctctcaccgctgagc
ggataaagaccccaccaaaacgtccaggaggccgcagaagaggacggctt
cccaataacagtagcaggcccagcacccccaccattaatgtgctggaatc
aaaggatacagacagtgatagggaagcagggactgaaacggggggagaga
acaatgataaagaagaagaagagaagaaagatgaaacttcgagctcctct
gaagcaaattctcggtgtcaaacaccaataaagatgaagccaaatattga
acctcctgagaatgtggagtggagtggtgctgaagcctcaatgtttagag
tcctcattggcacttactatgacaatttctgtgccattgctaggttaatt
gggaccaaaacatgtagacaggtgtatgagtttagagtcaaagaatctag
catcatagctccagctcccgctgaggatgtggatactcctccaaggaaaa
agaagaggaaacaccggttgtgggctgcacactgcagaaagatacagctg
aaaaaggacggctcctctaaccatgtttacaactatcaaccctgtgatca
tccacggcagccttgtgacagttcgtgcccttgtgtgatagcacaaaatt
tttgtgaaaagttttgtcaatgtagttcagagtgtcaaaaccgctttccg
ggatgccgctgcaaagcacagtgcaacaccaagcagtgcccgtgctacct
ggctgtccgagagtgtgaccctgacctctgtcttacttgtggagccgctg
accattgggacagtaaaaatgtgtcctgcaagaactgcagtattcagcgg
ggctccaaaaagcatctattgctggcaccatctgacgtggcaggctgggg
gatttttatcaaagatcctgtgcagaaaaatgaattcatctcagaatact
gtggagagattatttctcaagatgaagctgacagaagagggaaagtgtat
gataaatacatgtgcagctttctgttcaacttgaacaatgattttgtggt
ggatgcaacccgcaagggtaacaaaattcgttttgcaaatcattcggtaa
atccaaactgctatgcaaaagttatgatggttaacggtgatcacaggata
ggtatttttgccaagagagccatccagactggcgaagagctgttttttga
ttacagatacagccaggctgatgccctgaagtatgtcggcatcgaaagag
aaatggaaatcccttgacatctgctacctcctcccccctcctctgaaaca
gctgccttagcttcaggaacctcgagtactgtgggcaatttagaaaaaga
acatgcagtttgaaattctgaatttgcaaagtactgtaagaataatttat
agtaatgagtttaaaaatcaactttttattgccttctcaccagctgcaaa
gtgttttgtaccagtgaatttttgcaataatgcagtatggtacatttttc
aactttgaataaagaatacttgaacttgtccttgttgaatc

Full amino acid of EZH2, isoform b (GenBank
Accession No. NP 694543) (SEQ ID NO: 5)
MGQTGKKSEKGPVCWRKRVKSEYMRLRQLKRFRRADEVKSMFSSNRQKIL
ERTEILNQEWKQRRIQPVHILTSVSSLRGTREVEDETVLHNIPYMGDEVL
DQDGTFIEELIKNYDGKVHGDRECGFINDEIFVELVNALGQYNDDDDDDD
GDDPEEREEKQKDLEDHRDDKESRPPRKFPSDKIFEAISSMFPDKGTAEE
LKEKYKELTEQQLPGALPPECTPNIDGPNAKSVQREQSLHSFHTLFCRRC
FKYDCFLHPFHATPNTYKRKNTETALNKPCGPQCYQHLEGAKEFAAALTA
ERIKTPPKRPGGRRRGRLPNNSSRPSTPTINVLESKDTDSDREAGTETGG
ENNDKEEEEKKDETSSSSEANSRCQTPIKMKPNIEPPENVEWSGAEASMF
RVLIGTYYDNFCAIARLIGTKTCRQVYEFRVKESSIIAPAPAEDVDTPPR
KKKRKHRLWAAHCRKIQLKKDGSSNHVYNYQPCDHPRQPCDSSCPCVIAQ
NFCEKFCQCSSECQNRFPGCRCKAQCNTKQCPCYLAVRECDPDLCLTCGA
ADHWDSKNVSCKNCSIQRGSKKHLLLAPSDVAGWGIFIKDPVQKNEFISE
YCGEIISQDEADRRGKVYDKYMCSFLFNLNNDFVVDATRKGNKIR
FANHSVNPNCYAKVMMVNGDHRIGIFAKRAIQTGEELFFDYRYSQADALK
YVGIEREMEIP

Full amino acid of EZH2, isoform e (GenBank
Accession No. NP_001190178.1) (SEQ ID NO: 21)
MGQTGKKSEKGPVCWRKRVKSEYMRLRQLKRFRRADEVKSMFSSNRQKIL
ERTEILNQEWKQRRIQPVHILTSCSVTSDLDFPTQVIPLKTLNAVASVPI
MYSWSPLQQNFMVEDETVLHNIPYMGDEVLDQDGTFIEELIKNYDGKVHG
DRECGFINDEIFVELVNALGQYNDDDDDDDGDDPEEREEKQKDLEDHRDD
KESRPPRKFPSDKIFEAISSMFPDKGTAEELKEKYKELTEQQLPGALPPE
CTPNIDGPNAKSVQREQSLHSFHTLFCRRCFKYDCFLHPFHATPNTYKRK
NTETALDNKPCGPQCYQHLEGAKEFAAALTAERIKTPPKRPGGRRRGRLP
NNSSRPSTPTINVLESKDTDSDREAGTETGGENNDKEEEEKKDETSSSSE
ANSRCQTPIKMKPNIEPPENVEWSGAEASMFRVLIGTYYDNFCAIARLIG
TKTCRQVYEFRVKESSIIAPAPAEDVDTPPRKKKRKHRLWAAHCRKIQLK
KGQNRFPGCRCKAQCNTKQCPCYLAVRECDPDLCLTCGAADHWDSKNVSC
KNCSIQRGSKKHLLLAPSDVAGWGIFIKDPVQKNEFISEYCGEIISQDEA
DRRGKVYDKYMCSFLFNLNNDFVVDATRKGNKIRFANHSVNPNCYAKVMM
VNGDHRIGIFAKRAIQTGEELFFDYRYSQADALKYVGIEREMEIP

Homo sapiens enhancer of zeste homolog 2
(Drosophila) (EZH2), transcript variant 5, mRNA
(GenBank Accession No. NM 001203249.1) (SEQ ID
NO: 22)
GACGACGTTCGCGGCGGGGAACTCGGAGTAGCTTCGCCTCTGACGTTTCC
CCACGACGCACCCCGAAATCCCCCTGAGCTCCGGCGGTCGCGGGCTGCCC
TCGCCGCCTGGTCTGGCTTTATGCTAAGTTTGAGGGAAGAGTCGAGCTGC
TCTGCTCTCTATTGATTGTGTTTCTGGAGGGCGTCCTGTTGAATTCCCAC
TTCATTGTGTACATCCCCTTCCGTTCCCCCCAAAAATCTGTGCCACAGGG
TTACTTTTTGAAAGCGGGAGGAATCGAGAAGCACGATCTTTTGGAAAACT
TGGTGAACGCCTAAATAATCATGGGCCAGACTGGGAAGAAATCTGAGAAG
GGACCAGTTTGTTGGCGGAAGCGTGTAAAATCAGAGTACATGCGACTGAG
ACAGCTCAAGAGGTTCAGACGAGCTGATGAAGTAAAGAGTATGTTTAGTT
CCAATCGTCAGAAAATTTTGGAAAGAACGGAAATCTTAAACCAAGAATGG
AAACAGCGAAGGATACAGCCTGTGCACATCCTGACTTCTTGTTCGGTGAC
CAGTGACTTGGATTTTCCAACACAAGTCATCCCATTAAAGACTCTGAATG
CAGTTGCTTCAGTACCCATAATGTATTCTTGGTCTCCCCTACAGCAGAAT
TTTATGGTGGAAGATGAAACTGTTTTACATAACATTCCTTATATGGGAGA
TGAAGTTTTAGATCAGGATGGTACTTTCATTGAAGAACTAATAAAAAATT
ATGATGGGAAAGTACACGGGGATAGAGAATGTGGGTTTATAAATGATGAA
ATTTTTGTGGAGTTGGTGAATGCCCTTGGTCAATATAATGATGATGACGA
TGATGATGATGGAGACGATCCTGAAGAAAGAGAAGAAAAGCAGAAAGATC
TGGAGGATCACCGAGATGATAAAGAAAGCCGCCCACCTCGGAAATTTCCT
TCTGATAAAATTTTTGAAGCCATTTCCTCAATGTTTCCAGATAAGGGCAC
AGCAGAAGAACTAAAGGAAAAATATAAAGAACTCACCGAACAGCAGCTCC
CAGGCGCACTTCCTCCTGAATGTACCCCCAACATAGATGGACCAAATGCT
AAATCTGTTCAGAGAGAGCAAAGCTTACACTCCTTTCATACGCTTTTCTG
TAGGCGATGTTTTAAATATGACTGCTTCCTACATCCTTTTCATGCAACAC
CCAACACTTATAAGCGGAAGAACACAGAAACAGCTCTAGACAACAAACCT
TGTGGACCACAGTGTTACCAGCATTTGGAGGGAGCAAAGGAGTTTGCTGC
TGCTCTCACCGCTGAGCGGATAAAGACCCCACCAAAACGTCCAGGAGGCC
GCAGAAGAGGACGGCTTCCCAATAACAGTAGCAGGCCCAGCACCCCCACC
ATTAATGTGCTGGAATCAAAGGATACAGACAGTGATAGGGAAGCAGGGAC
TGAAACGGGGGGAGAGAACAATGATAAAGAAGAAGAAGAGAAGAAAGATG
AAACTTCGAGCTCCTCTGAAGCAAATTCTCGGTGTCAAACACCAATAAAG
ATGAAGCCAAATATTGAACCTCCTGAGAATGTGGAGTGGAGTGGTGCTGA
AGCCTCAATGTTTAGAGTCCTCATTGGCACTTACTATGACAATTTCTGTG
CCATTGCTAGGTTAATTGGGACCAAAACATGTAGACAGGTGTATGAGTTT
AGAGTCAAAGAATCTAGCATCATAGCTCCAGCTCCCGCTGAGGATGTGGA
TACTCCTCCAAGGAAAAAGAAGAGGAAACACCGGTTGTGGGCTGCACACT
GCAGAAAGATACAGCTGAAAAAGGGTCAAAACCGCTTTCCGGGATGCCGC
TGCAAAGCACAGTGCAACACCAAGCAGTGCCCGTGCTACCTGGCTGTCCG
AGAGTGTGACCCTGACCTCTGTCTTACTTGTGGAGCCGCTGACCATTGGG
ACAGTAAAAATGTGTCCTGCAAGAACTGCAGTATTCAGCGGGGCTCCAAA
AAGCATCTATTGCTGGCACCATCTGACGTGGCAGGCTGGGGGATTTTTAT
CAAAGATCCTGTGCAGAAAAATGAATTCATCTCAGAATACTGTGGAGAGA
TTATTTCTCAAGATGAAGCTGACAGAAGAGGGAAAGTGTATGATAAATAC
ATGTGCAGCTTTCTGTTCAACTTGAACAATGATTTTGTGGTGGATGCAAC
CCGCAAGGGTAACAAAATTCGTTTTGCAAATCATTCGGTAAATCCAAACT
GCTATGCAAAAGTTATGATGGTTAACGGTGATCACAGGATAGGTATTTTT
GCCAAGAGAGCCATCCAGACTGGCGAAGAGCTGTTTTTTGATTACAGATA
CAGCCAGGCTGATGCCCTGAAGTATGTCGGCATCGAAAGAGAAATGGAAA
TCCCTTGACATCTGCTACCTCCTCCCCCCTCCTCTGAAACAGCTGCCTTA
GCTTCAGGAACCTCGAGTACTGTGGGCAATTTAGAAAAAGAACATGCAGT
TTGAAATTCTGAATTTGCAAAGTACTGTAAGAATAATTTATAGTAATGAG
TTTAAAAATCAACTTTTTATTGCCTTCTCACCAGCTGCAAAGTGTTTTGT
ACCAGTGAATTTTTGCAATAATGCAGTATGGTACATTTTTCAACTTTGAA
TAAAGAATACTTGAACTTGTCCTTGTTGAATC

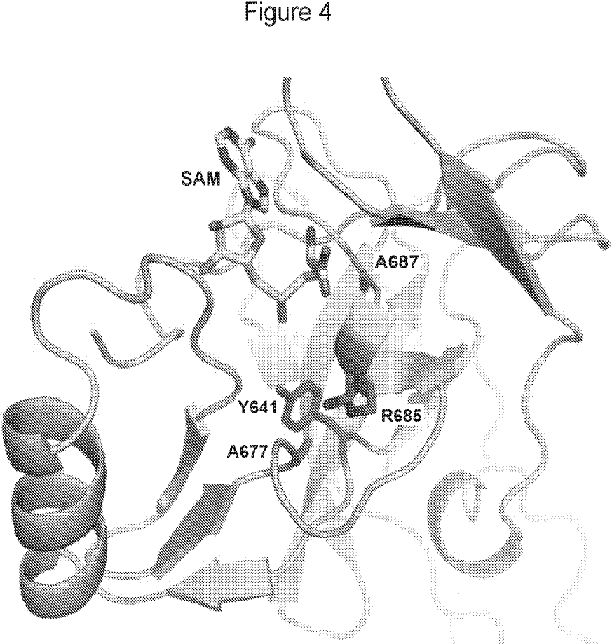

A structure model of partial EZH2 protein based on the A chain of nuclear receptor binding SET domain protein 1 (NSD1) is provided in FIG. 4. This model corresponds to amino acid residues 533-732 of EZH2 sequence of SEQ ID NO: 1.

The corresponding amino acid sequence of this structure model is provided below. The residues in the substrate pocket domain are underlined. The residues in the SET domain are shown italic.

SCPCVIAQNFCEKFCQCSSECQNRFPGCRCKAQCNTKQCPCYLAVRECDP
DLCLTCGAADHWDSKNVSCKNCSIQRGSKKHLLLAPSDIlAGWGIFIKDP
VQKNEFISEY⁶⁴¹CGEIISQDEADRRGKVYDKYMCSFLFNLNNDFV⁶⁷⁴V
DA⁶⁷⁷TRKGNKIR⁶⁸⁵FA⁶⁸⁷NHSVNPNCYAKVMMVNGDHRIGIFAKRAI
QTGEELFFDYRYSQAD

(SEQ ID NO: 6).

The catalytic site of EZH2 is believed to reside in a conserved domain of the protein known as the SET domain. The amino acid sequence of the SET domain of EZH2 is provided by the following partial sequence spanning amino acid residues 613-726 of Swiss-Prot Accession No. Q15910 (SEQ ID NO: 1):

HLLLAPSDVAGWGIFIKDPVQKNEFISEYCGEIISQDEADRRGKVYDKYM
CSFLFNLNNDFVVDATRKGNKIRFANHSVNPNCYAKVMMVNGDHRIGIFA
KRAIQTGEELFFDY

(SEQ ID NO: 7).

The tyrosine (Y) residue shown underlined in SEQ ID NO: 7 is Tyr641 (Y641) in Swiss-Prot Accession No. Q15910 (SEQ ID NO: 1).

The SET domain of GenBank Accession No. NP_004447 (SEQ ID NO: 3) spans amino acid residues 618-731 and is identical to SEQ ID NO:6. The tyrosine residue corresponding to Y641 in Swiss-Prot Accession No. Q15910 shown underlined in SEQ ID NO: 7 is Tyr646 (Y646) in GenBank Accession No. NP_004447 (SEQ ID NO: 3).

The SET domain of GenBank Accession No. NP_694543 (SEQ ID NO: 5) spans amino acid residues 574-687 and is identical to SEQ ID NO: 7. The tyrosine residue corresponding to Y641 in Swiss-Prot Accession No. Q15910 shown underlined in SEQ ID NO: 7 is Tyr602 (Y602) in GenBank Accession No. NP_694543 (SEQ ID NO: 5).

The nucleotide sequence encoding the SET domain of GenBank Accession No. NP_004447 is

catctattgctggcaccatctgacgtggcaggctgggggatttttatcaa
agatcctgtgcagaaaaatgattcatctcagaatactgtggagagattat
ttctcaagatgaagctgacagaagagggaaagtgtatgataaatacatgt
gcagctttctgttcaacttgaacaatgattttgtggtggatgcaacccgc
aagggtaacaaaattcgttttgcaaatcattcggtaaatccaaactgcta
tgcaaaagttatgatggttaacggtgatcacaggataggtatttttgcca
agagagccatccagactggcgaagagctgttttttgattac

(SEQ ID NO: 8), where the codon encoding Y641 is shown underlined.

For purposes of this application, amino acid residue Y641 of human EZH2 is to be understood to refer to the tyrosine residue that is or corresponds to Y641 in Swiss-Prot Accession No. Q15910.

Full amino acid sequence of Y641 mutant EZH2
(SEQ ID NO: 9)
MGQTGKKSEKGPVCWRKRVKSEYMRLRQLKRFRRADEVKSMFSSNRQKIL
ERTEILNQEWKQRRIQPVHILTSVSSLRGTRECSVTSDLDFPTQVIPLKT
LNAVASVPIMYSWSPLQQNFMVEDETVLHNIPYMGDEVLDQDGTFIEELI
KNYDGKVHGDRECGFINDEIFVELVNALGQYNDDDDDDDGDDPEEREEKQ
KDLEDHRDDKESRPPRKFPSDKIFEAISSMFPDKGTAEELKEKYKELTEQ
QLPGALPPECTPNIDGPNAKSVQREQSLHSFHTLFCRRCFKYDCFLHPFH
ATPNTYKRKNTETALDNKPCGPQCYQHLEGAKEFAAALTAERIKTPPKRP
GGRRRGRLPNNSSRPSTPTINVLESKDTDSDREAGTETGGENNDKEEEEK
KDETSSSSEANSRCQTPIKMKPNIEPPENVEWSGAEASMFRVLIGTYYDN
FCAIARLIGTKTCRQVYEFRVKESSIIAPAPAEDVDTPPRKKKRKHRLWA
AHCRKIQLKKDGSSNHVYNYQPCDHPRQPCDSSCPCVIAQNFCEKFCQCS
SECQNRFPGCRCKAQCNTKQCPCYLAVRECDPDLCLTCGAADHWDSKNVS
CKNCSIQRGSKKHLLLAPSDVAGWGIFIKDPVQKNEFISEXCGEIISQDE
ADRRGKVYDKYMCSFLFNLNNDFVVDATRKGNKIRFANHSVNPNCYAKVM
MVNGDHRIGIFAKRAIQTGEELFFDYRYSQADALKYVGIEREMEIP
Wherein x can be any amino acid
residue other than tyrosine (Y)

A Y641 mutant of human EZH2, and, equivalently, a Y641 mutant of EZH2, is to be understood to refer to a human EZH2 in which the amino acid residue corresponding to Y641 of wild-type human EZH2 is substituted by an amino acid residue other than tyrosine.

In one embodiment the amino acid sequence of a Y641 mutant of EZH2 differs from the amino acid sequence of wild-type human EZH2 only by substitution of a single amino acid residue corresponding to Y641 of wild-type human EZH2 by an amino acid residue other than tyrosine.

In one embodiment the amino acid sequence of a Y641 mutant of EZH2 differs from the amino acid sequence of wild-type human EZH2 only by substitution of phenylalanine (F) for the single amino acid residue corresponding to Y641 of wild-type human EZH2. The Y641 mutant of EZH2 according to this embodiment is referred to herein as a Y641F mutant or, equivalently, Y641F.

Y641F (SEQ ID NO: 10)
MGQTGKKSEKGPVCWRKRVKSEYMRLRQLKRFRRADEVKSMFSSNRQKIL
ERTEILNQEWKQRRIQPVHILTSVSSLRGTRECSVTSDLDFPTQVIPLKT
LNAVASVPIMYSWSPLQQNFMVEDETVLHNIPYMGDEVLDQDGTFIEELI
KNYDGKVHGDRECGFINDEIFVELVNALGQYNDDDDDDDGDDPEEREEKQ
KDLEDHRDDKESRPPRKFPSDKIFEAISSMFPDKGTAEELKEKYKELTEQ
QLPGALPPECTPNIDGPNAKSVQREQSLHSFHTLFCRRCFKYDCFLHPFH
ATPNTYKRKNTETALDNKPCGPQCYQHLEGAKEFAAALTAERIKTPPKRP
GGRRRGRLPNNSSRPSTPTINVLESKDTDSDREAGTETGGENNDKEEEEK
KDETSSSSEANSRCQTPIKMKPNIEPPENVEWSGAEASMFRVLIGTYYDN
FCAIARLIGTKTCRQVYEFRVKESSIIAPAPAEDVDTPPRKKKRKHRLWA
AHCRKIQLKKDGSSNHVYNYQPCDHPRQPCDSSCPCVIAQNFCEKFCQCS
SECQNRFPGCRCKAQCNTKQCPCYLAVRECDPDLCLTCGAADHWDSKNVS
CKNCSIQRGSKKHLLLAPSDVAGWGIFIKDPVQKNEFISEFCGEIISQDE
ADRRGKVYDKYMCSFLFNLNNDFVVDATRKGNKIRFANHSVNPNCYAKVM
MVNGDHRIGIFAKRAIQTGEELFFDYRYSQADALKYVGIEREMEIP

In one embodiment the amino acid sequence of a Y641 mutant of EZH2 differs from the amino acid sequence of wild-type human EZH2 only by substitution of histidine (H) for the single amino acid residue corresponding to Y641 of wild-type human EZH2. The Y641 mutant of EZH2 according to this embodiment is referred to herein as a Y641H mutant or, equivalently, Y641H.

Y641H (SEQ ID NO: 11)
MGQTGKKSEKGPVCWRKRVKSEYMRLRQLKRFRRADEVKSMFSSNRQKIL
ERTEILNQEWKQRRIQPVHILTSVSSLRGTRECSVTSDLDFPTQVIPLKT
LNAVASVPIMYSWSPLQQNFMVEDETVLHNIPYMGDEVLDQDGTFIEELI
KNYDGKVHGDRECGFINDEIFVELVNALGQYNDDDDDDDGDDPEEREEKQ
KDLEDHRDDKESRPPRKFPSDKIFEAISSMFPDKGTAEELKEKYKELTEQ
QLPGALPPECTPNIDGPNAKSVQREQSLHSFHTLFCRRCFKYDCFLHPFH
ATPNTYKRKNTETALDNKPCGPQCYQHLEGAKEFAAALTAERIKTPPKRP
GGRRRGRLPNNSSRPSTPTINVLESKDTDSDREAGTETGGENNDKEEEEK
KDETSSSSEANSRCQTPIKMKPNIEPPENVEWSGAEASMFRVLIGTYYDN
FCAIARLIGTKTCRQVYEFRVKESSIIAPAPAEDVDTPPRKKKRKHRLWA
AHCRKIQLKKDGSSNHVYNYQPCDHPRQPCDSSCPCVIAQNFCEKFCQCS
SECQNRFPGCRCKAQCNTKQCPCYLAVRECDPDLCLTCGAADHWDSKNVS
CKNCSIQRGSKKHLLLAPSDVAGWGIFIKDPVQKNEFISEHCGEIISQDE
ADRRGKVYDKYMCSFLFNLNNDFVVDATRKGNKIRFANHSVNPNCYAKVM
MVNGDHRIGIFAKRAIQTGEELFFDYRYSQADALKYVGIEREMEIP

In one embodiment the amino acid sequence of a Y641 mutant of EZH2 differs from the amino acid sequence of wild-type human EZH2 only by substitution of asparagine (N) for the single amino acid residue corresponding to Y641 of wild-type human EZH2. The Y641 mutant of EZH2 according to this embodiment is referred to herein as a Y641N mutant or, equivalently, Y641N.

Y641N (SEQ ID NO: 12)
MGQTGKKSEKGPVCWRKRVKSEYMRLRQLKRFRRADEVKSMFSSNRQKIL
ERTEILNQEWKQRRIQPVHILTSVSSLRGTRECSVTSDLDFPTQVIPLKT
LNAVASVPIMYSWSPLQQNFMVEDETVLHNIPYMGDEVLDQDGTFIEELI
KNYDGKVHGDRECGFINDEIFVELVNALGQYNDDDDDDDGDDPEEREEKQ
KDLEDHRDDKESRPPRKFPSDKIFEAISSMFPDKGTAEELKEKYKELTEQ
QLPGALPPECTPNIDGPNAKSVQREQSLHSFHTLFCRRCFKYDCFLHPFH
ATPNTYKRKNTETALDNKPCGPQCYQHLEGAKEFAAALTAERIKTPPKRP
GGRRRGRLPNNSSRPSTPTINVLESKDTDSDREAGTETGGENNDKEEEEK
KDETSSSSEANSRCQTPIKMKPNIEPPENVEWSGAEASMFRVLIGTYYDN
FCAIARLIGTKTCRQVYEFRVKESSIIAPAPAEDVDTPPRKKKRKHRLWA
AHCRKIQLKKDGSSNHVYNYQPCDHPRQPCDSSCPCVIAQNFCEKFCQCS
SECQNRFPGCRCKAQCNTKQCPCYLAVRECDPDLCLTCGAADHWDSKNVS
CKNCSIQRGSKKHLLLAPSDVAGWGIFIKDPVQKNEFISENCGEIISQDE
ADRRGKVYDKYMCSFLFNLNNDFVVDATRKGNKIRFANHSVNPNCYAKVM
MVNGDHRIGIFAKRAIQTGEELFFDYRYSQADALKYVGIEREMEIP

In one embodiment the amino acid sequence of a Y641 mutant of EZH2 differs from the amino acid sequence of wild-type human EZH2 only by substitution of serine (S) for the single amino acid residue corresponding to Y641 of wild-type human EZH2. The Y641 mutant of EZH2 according to this embodiment is referred to herein as a Y641S mutant or, equivalently, Y641S.

Y641S (SEQ ID NO: 13)
MGQTGKKSEKGPVCWRKRVKSEYMRLRQLKRFRRADEVKSMFSSNRQKIL
ERTEILNQEWKQRRIQPVHILTSVSSLRGTRECSVTSDLDFPTQVIPLKT
LNAVASVPIMYSWSPLQQNFMVEDETVLHNIPYMGDEVLDQDGTFIEELI
KNYDGKVHGDRECGFINDEIFVELVNALGQYNDDDDDDDGDDPEEREEKQ
KDLEDHRDDKESRPPRKFPSDKIFEAISSMFPDKGTAEELKEKYKELTEQ
QLPGALPPECTPNIDGPNAKSVQREQSLHSFHTLFCRRCFKYDCFLHPFH
ATPNTYKRKNTETALDNKPCGPQCYQHLEGAKEFAAALTAERIKTPPKRP
GGRRRGRLPNNSSRPSTPTINVLESKDTDSDREAGTETGGENNDKEEEEK
KDETSSSSEANSRCQTPIKMKPNIEPPENVEWSGAEASMFRVLIGTYYDN
FCAIARLIGTKTCRQVYEFRVKESSIIAPAPAEDVDTPPRKKKRKHRLWA
AHCRKIQLKKDGSSNHVYNYQPCDHPRQPCDSSCPCVIAQNFCEKFCQCS
SECQNRFPGCRCKAQCNTKQCPCYLAVRECDPDLCLTCGAADHWDSKNVS
CKNCSIQRGSKKHLLLAPSDVAGWGIFIKDPVQKNEFISESCGEIISQDE
ADRRGKVYDKYMCSFLFNLNNDFVVDATRKGNKIRFANHSVNPNCYAKVM
MVNGDHRIGIFAKRAIQTGEELFFDYRYSQADALKYVGIEREMEIP

In one embodiment the amino acid sequence of a Y641 mutant of EZH2 differs from the amino acid sequence of wild-type human EZH2 only by substitution of cysteine (C) for the single amino acid residue corresponding to Y641 of wild-type human EZH2. The Y641 mutant of EZH2 according to this embodiment is referred to herein as a Y641C mutant or, equivalently, Y641C.

Y641C (SEQ ID NO: 14)
MGQTGKKSEKGPVCWRKRVKSEYMRLRQLKRFRRADEVKSMFSSNRQKIL
ERTEILNQEWKQRRIQPVHILTSVSSLRGTRECSVTSDLDFPTQVIPLKT
LNAVASVPIMYSWSPLQQNFMVEDETVLHNIPYMGDEVLDQDGTFIEELI
KNYDGKVHGDRECGFINDEIFVELVNALGQYNDDDDDDDGDDPEEREEKQ
KDLEDHRDDKESRPPRKFPSDKIFEAISSMFPDKGTAEELKEKYKELTEQ
QLPGALPPECTPNIDGPNAKSVQREQSLHSFHTLFCRRCFKYDCFLHPFH
ATPNTYKRKNTETALDNKPCGPQCYQHLEGAKEFAAALTAERIKTPPKRP
GGRRRGRLPNNSSRPSTPTINVLESKDTDSDREAGTETGGENNDKEEEEK
KDETSSSSEANSRCQTPIKMKPNIEPPENVEWSGAEASMFRVLIGTYYDN
FCAIARLIGTKTCRQVYEFRVKESSIIAPAPAEDVDTPPRKKKRKHRLWA
AHCRKIQLKKDGSSNHVYNYQPCDHPRQPCDSSCPCVIAQNFCEKFCQCS
SECQNRFPGCRCKAQCNTKQCPCYLAVRECDPDLCLTCGAADHWDSKNVS
CKNCSIQRGSKKHLLLAPSDVAGWGIFIKDPVQKNEFISECCGEIISQDE
ADRRGKVYDKYMCSFLFNLNNDFVVDATRKGNKIRFANHSVNPNCYAKVM
MVNGDHRIGIFAKRAIQTGEELFFDYRYSQADALKYVGIEREMEIP

In one embodiment the amino acid sequence of a A677 mutant of EZH2 differs from the amino acid sequence of wild-type human EZH2 only by substitution of a non-alanine amino acid, preferably glycine (G) for the single amino acid residue corresponding to A677 of wild-type human EZH2. The A677 mutant of EZH2 according to this embodiment is referred to herein as an A677 mutant, and preferably an A677G mutant or, equivalently, A677G.

A677 (SEQ ID NO: 15)
MGQTGKKSEKGPVCWRKRVKSEYMRLRQLKRFRRADEVKSMFSSNRQKIL
ERTEILNQEWKQRRIQPVHILTSVSSLRGTRECSVTSDLDFPTQVIPLKT
LNAVASVPIMYSWSPLQQNFMVEDETVLHNIPYMGDEVLDQDGTFIEELI
KNYDGKVHGDRECGFINDEIFVELVNALGQYNDDDDDDDGDDPEEREEKQ
KDLEDHRDDKESRPPRKFPSDKIFEAISSMFPDKGTAEELKEKYKELTEQ
QLPGALPPECTPNIDGPNAKSVQREQSLHSFHTLFCRRCFKYDCFLHPFH
ATPNTYKRKNTETALDNKPCGPQCYQHLEGAKEFAAALTAERIKTPPKRP
GGRRRGRLPNNSSRPSTPTINVLESKDTDSDREAGTETGGENNDKEEEEK
KDETSSSSEANSRCQTPIKMKPNIEPPENVEWSGAEASMFRVLIGTYYDN
FCAIARLIGTKTCRQVYEFRVKESSIIAPAPAEDVDTPPRKKKRKHRLWA
AHCRKIQLKKDGSSNHVYNYQPCDHPRQPCDSSCPCVIAQNFCEKFCQCS
SECQNRFPGCRCKAQCNTKQCPCYLAVRECDPDLCLTCGAADHWDSKNVS
CKNCSIQRGSKKHLLLAPSDVAGWGIFIKDPVQKNEFISEYCGEIISQDE
ADRRGKVYDKYMCSFLFNLNNDFVVDXTRKGNKIRFANHSVNPNCYAKVM
MVNGDHRIGIFAKRAIQTGEELFFDYRYSQADALKYVGIEREMEIP
Wherein X is preferably a glycine (G).

In one embodiment the amino acid sequence of a A687 mutant of EZH2 differs from the amino acid sequence of wild-type human EZH2 only by substitution of a non-alanine amino acid, preferably valine (V) for the single amino acid residue corresponding to A687 of wild-type human EZH2. The A687 mutant of EZH2 according to this embodiment is referred to herein as an A687 mutant and preferably an A687V mutant or, equivalently, A687V.

A687 (SEQ ID NO: 16)
MGQTGKKSEKGPVCWRKRVKSEYMRLRQLKRFRRADEVKSMFSSNRQKIL
ERTEILNQEWKQRRIQPVHILTSVSSLRGTRECSVTSDLDFPTQVIPLKT
LNAVASVPIMYSWSPLQQNFMVEDETVLHNIPYMGDEVLDQDGTFIEELI
KNYDGKVHGDRECGFINDEIFVELVNALGQYNDDDDDDDGDDPEEREEKQ
KDLEDHRDDKESRPPRKFPSDKIFEAISSMFPDKGTAEELKEKYKELTEQ
QLPGALPPECTPNIDGPNAKSVQREQSLHSFHTLFCRRCFKYDCFLHPFH
ATPNTYKRKNTETALDNKPCGPQCYQHLEGAKEFAAALTAERIKTPPKRP
GGRRRGRLPNNSSRPSTPTINVLESKDTDSDREAGTETGGENNDKEEEEK
KDETSSSSEANSRCQTPIKMKPNIEPPENVEWSGAEASMFRVLIGTYYDN
FCAIARLIGTKTCRQVYEFRVKESSIIAPAPAEDVDTPPRKKKRKHRLWA
AHCRKIQLKKDGSSNHVYNYQPCDHPRQPCDSSCPCVIAQNFCEKFCQCS
SECQNRFPGCRCKAQCNTKQCPCYLAVRECDPDLCLTCGAADHWDSKNVS
CKNCSIQRGSKKHLLLAPSDVAGWGIFIKDPVQKNEFISEYCGEIISQDE
ADRRGKVYDKYMCSFLFNLNNDFVVDATRKGNKIRFXNHSVNPNCYAKVM
MVNGDHRIGIFAKRAIQTGEELFFDYRYSQADALKYVGIEREMEIP
Wherein X is preferably a valine (V).

In one embodiment the amino acid sequence of a R685 mutant of EZH2 differs from the amino acid sequence of wild-type human EZH2 only by substitution of a non-arginine amino acid, preferably histidine (H) or cysteine (C) for the single amino acid residue corresponding to R685 of wild-type human EZH2. The R685 mutant of EZH2 according to this embodiment is referred to herein as an R685 mutant and preferably an R685C mutant or an R685H mutant or, equivalently, R685H or R685C.

A685 (SEQ ID NO: 17)
MGQTGKKSEKGPVCWRKRVKSEYMRLRQLKRFRRADEVKSMFSSNRQKIL
ERTEILNQEWKQRRIQPVHILTSVSSLRGTRECSVTSDLDFPTQVIPLKT
LNAVASVPIMYSWSPLQQNFMVEDETVLHNIPYMGDEVLDQDGTFIEELI
KNYDGKVHGDRECGFINDEIFVELVNALGQYNDDDDDDDGDDPEEREEKQ
KDLEDHRDDKESRPPRKFPSDKIFEAISSMFPDKGTAEELKEKYKELTEQ
QLPGALPPECTPNIDGPNAKSVQREQSLHSFHTLFCRRCFKYDCFLHPFH
ATPNTYKRKNTETALDNKPCGPQCYQHLEGAKEFAAALTAERIKTPPKRP
GGRRRGRLPNNSSRPSTPTINVLESKDTDSDREAGTETGGENNDKEEEEK
KDETSSSSEANSRCQTPIKMKPNIEPPENVEWSGAEASMFRVLIGTYYDN
FCAIARLIGTKTCRQVYEFRVKESSIIAPAPAEDVDTPPRKKKRKHRLWA
AHCRKIQLKKDGSSNHVYNYQPCDHPRQPCDSSCPCVIAQNFCEKFCQCS
SECQNRFPGCRCKAQCNTKQCPCYLAVRECDPDLCLTCGAADHWDSKNVS
CKNCSIQRGSKKHLLLAPSDVAGWGIFIKDPVQKNEFISEYCGEIISQDE
ADRRGKVYDKYMCSFLFNLNNDFVVDATRKGNKIXFANHSVNPNCYAKVM
MVNGDHRIGIFAKRAIQTGEELFFDYRYSQADALKYVGIEREMEIP
Wherein X is preferably a cysteine (C)
or a histidine (H).

In one embodiment the amino acid sequence of a mutant of EZH2 differs from the amino acid sequence of wild-type human EZH2 in one or more amino acid residues in its substrate pocket domain as defined in SEQ ID NO: 6. The mutant of EZH2 according to this embodiment is referred to herein as an EZH2 mutant.

Mutant EZH2 comprising one or more mutations in
the substrate pocket domain (SEQ ID NO: 18)
MGQTGKKSEKGPVCWRKRVKSEYMRLRQLKRFRRADEVKSMFSSNRQKIL
ERTEILNQEWKQRRIQPVHILTSVSSLRGTRECSVTSDLDFPTQVIPLKT
LNAVASVPIMYSWSPLQQNFMVEDETVLHNIPYMGDEVLDQDGTFIEELI
KNYDGKVHGDRECGFINDEIFVELVNALGQYNDDDDDDDGDDPEEREEKQ
KDLEDHRDDKESRPPRKFPSDKIFEAISSMFPDKGTAEELKEKYKELTEQ
QLPGALPPECTPNIDGPNAKSVQREQSLHSFHTLFCRRCFKYDCFLHPFH
ATPNTYKRKNTETALDNKPCGPQCYQHLEGAKEFAAALTAERIKTPPKRP
GGRRRGRLPNNSSRPSTPTINVLESKDTDSDREAGTETGGENNDKEEEEK
KDETSSSSEANSRCQTPIKMKPNIEPPENVEWSGAEASMFRVLIGTYYDN
FCAIARLIGTKTCRQVYEFRVKESSIIAPAPAEDVDTPPRKKKRKHRLWA
AHCRKIQLKKDGSSNHVYNYQPCDHPRQPCDSSCPCVIAQNFCEKFCQCS
SECQNRFPGCRCKAQCNTKQCPCYLAVRECDPDLCLTCGAADHWDSKNVS
CKNCSIQRGSKKHLLLAPSDVAGWGIFIKDPVQKNEFISEXCGEIISQDE
ADRRGKVYDKYMXXXLXNLNNDFXXDXTRKGNKXXXXHSVNPNCYAKVMM
VNGDHRXGIFAKRAIQTGEELFXDXRYSXADALKYVGIEREMEIP
Wherein X can be any amino acid except
the corresponding wild type residue.

### Histone Acetyltransferases

Histone acetyltransferase (HAT) enzymes of the disclosure activate gene transcription by transferring an acetyl group from acetyl CoA to form ε-N-acetyllysine, which serves to modify histones and increase transcription by, for example, generating or exposing binding sites for protein-protein interaction domains.

HAT enzymes of the disclosure include, but are not limited to, those enzymes of the p300/CBP family.

In certain embodiments, a mutation of the disclosure may occur in a sequence encoding the p300 HAT, including the nucleotide sequence of the EP300 gene, encoding p300 (below, corresponding to GenBank Accession No. NM_001429.3, defined as Homo sapiens E1A binding protein p300 (EP300), mRNA; and identified as SEQ ID NO: 19).

1 GCCGAGGAGG AAGAGGTTGA TGGCGGCGGC GGAGCTCCGA GAGACCTCGG CTGGGCAGGG
61 GCCGGCCGTG GCGGGCCGGG GACTGCGCCT CTAGAGCCGC GAGTTCTCGG GAATTCGCCG
121 CAGCGGACGC GCTCGGCGAA TTTGTGCTCT TGTGCCCTCC TCCGGGCTTG GGCCCAGGCC
181 CGGCCCCTCG CACTTGCCCT TACCTTTTCT ATCGAGTCCG CATCCCTCTC CAGCCACTGC
241 GACCCGGCGA AGAGAAAAAG GAACTTCCCC CACCCCCTCG GGTGCCGTCG GAGCCCCCCA
301 GCCCACCCCT GGGTGCGGCG CGGGGACCCC GGGCCGAAGA AGAGATTTCC TGAGGATTCT
361 GGTTTTCCTC GCTTGTATCT CCGAAAGAAT TAAAAATGGC CGAGAATGTG GTGGAACCGG
421 GGCCGCCTTC AGCCAAGCGG CCTAAACTCT CATCTCCGGC CCTCTCGGCG TCCGCCAGCG
481 ATGGCACAGA TTTTGGCTCT CTATTTGACT TGGAGCACGA CTTACCAGAT GAATTAATCA
541 ACTCTACAGA ATTGGGACTA ACCAATGGTG GTGATATTAA TCAGCTTCAG ACAAGTCTTG
601 GCATGGTACA AGATGCAGCT TCTAAACATA AACAGCTGTC AGAATTGCTG CGATCTGGTA
661 GTTCCCCTAA CCTCAATATG GGAGTTGGTG GCCCAGGTCA AGTCATGGCC AGCCAGGCCC
721 AACAGAGCAG TCCTGGATTA GGTTTGATAA ATAGCATGGT CAAAAGCCCA ATGACACAGG
781 CAGGCTTGAC TTCTCCCAAC ATGGGGATGG GCACTAGTGG ACCAAATCAG GGTCCTACGC
841 AGTCAACAGG TATGATGAAC AGTCCAGTAA ATCAGCCTGC CATGGGAATG AACACAGGGA
901 TGAATGCGGG CATGAATCCT GGAATGTTGG CTGCAGGCAA TGGACAAGGG ATAATGCCTA
961 ATCAAGTCAT GAACGGTTCA ATTGGAGCAG GCCGAGGGCG ACAGAATATG CAGTACCCAA
1021 ACCCAGGCAT GGGAAGTGCT GGCAACTTAC TGACTGAGCC TCTTCAGCAG GGCTCTCCCC
1081 AGATGGGAGG ACAAACAGGA TTGAGAGGCC CCCAGCCTCT TAAGATGGGA ATGATGAACA
1141 ACCCCAATCC TTATGGTTCA CCATATACTC AGAATCCTGG ACAGCAGATT GGAGCCAGTG
1201 GCCTTGGTCT CCAGATTCAG ACAAAAACTG TACTATCAAA TAACTTATCT CCATTTGCTA
1261 TGGACAAAAA GGCACTTCCT GGTGGAGGAA TGCCCAACAT GGGTCAACAG CCAGCCCCGC
1321 AGGTCCAGCA GCCAGGCCTG GTGACTCCAG TTGCCCAAGG GATGGGTTCT GGAGCACATA
1381 CAGCTGATCC AGAGAAGCGC AAGCTCATCC AGCAGCAGCT TGTTCTCCTT TTGCATGCTC
1441 ACAAGTGCCA GCGCCGGGAA CAGGCCAATG GGGAAGTGAG GCAGTGCAAC CTTCCCCACT
1501 GTCGCACAAT GAAGAATGTC CTAAACCACA TGACACACTG CCAGTCAGGC AAGTCTTGCC
1561 AAGTGGCACA CTGTGCATCT TCTCGACAAA TCATTTCACA CTGGAAGAAT TGTACAAGAC
1621 ATGATTGTCC TGTGTGTCTC CCCCTCAAAA ATGCTGGTGA TAAGAGAAAT CAACAGCCAA
1681 TTTTGACTGG AGCACCCGTT GGACTTGGAA ATCCTAGCTC TCTAGGGGTG GGTCAACAGT
1741 CTGCCCCCAA CCTAAGCACT GTTAGTCAGA TTGATCCCAG CTCCATAGAA AGAGCCTATG
1801 CAGCTCTTGG ACTACCCTAT CAAGTAAATC AGATGCCGAC ACAACCCCAG GTGCAAGCAA
1861 AGAACCAGCA GAATCAGCAG CCTGGGCAGT CTCCCCAAGG CATGCGGCCC ATGAGCAACA
1921 TGAGTGCTAG TCCTATGGGA GTAAATGGAG GTGTAGGAGT TCAAACGCCG AGTCTTCTTT
1981 CTGACTCAAT GTTGCATTCA GCCATAAATT CTCAAAACCC AATGATGAGT GAAAATGCCA
2041 GTGTGCCCTC CCTGGGTCCT ATGCCAACAG CAGCTCAACC ATCCACTACT GGAATTCGGA
2101 AACAGTGGCA CGAAGATATT ACTCAGGATC TTCGAAATCA TCTTGTTCAC AAACTCGTCC
2161 AAGCCATATT TCCTACGCCG GATCCTGCTG CTTTAAAAGA CAGACGGATG GAAAACCTAG
2221 TTGCATATGC TCGGAAAGTT GAAGGGGACA TGTATGAATC TGCAAACAAT CGAGCGGAAT
2281 ACTACCACCT TCTAGCTGAG AAAATCTATA AGATCCAGAA AGAACTAGAA GAAAAACGAA
2341 GGACCAGACT ACAGAAGCAG AACATGCTAC CAAATGCTGC AGGCATGGTT CCAGTTTCCA
2401 TGAATCCAGG GCCTAACATG GGACAGCCGC AACCAGGAAT GACTTCTAAT GGCCCTCTAC
2461 CTGACCCAAG TATGATCCGT GGCAGTGTGC CAAACCAGAT GATGCCTCGA ATAACTCCAC
2521 AATCTGGTTT GAATCAATTT GGCCAGATGA GCATGGCCCA GCCCCCTATT GTACCCCGGC
2851 AAACCCCTCC TCTTCAGCAC CATGGACAGT TGGCTCAACC TGGAGCTCTC AACCCGCCTA
2641 TGGGCTATGG GCCTCGTATG CAACAGCCTT CCAACCAGGG CCAGTTCCTT CCTCAGACTC
2701 AGTTCCCATC ACAGGGAATG AATGTAACAA ATATCCCTTT GGCTCCGTCC AGCGGTCAAG
2761 CTCCAGTGTC TCAAGCACAA ATGTCTAGTT CTTCCTGCCC GGTGAACTCT CCTATAATGC
2821 CTCCAGGGTC TCAGGGGAGC CACATTCACT GTCCCCAGCT TCCTCAACCA GCTCTTCATC
2881 AGAATTCACC CTCGCCTGTA CCTAGTCGTA CCCCCACCCC TCACCATACT CCCCCAAGCA
2941 TAGGGGCTCA GCAGCCACCA GCAACAACAA TTCCAGCCCC TGTTCCTACA CCTCCTGCCA
3001 TGCCACCTGG GCCACAGTCC CAGGCTCTAC ATCCCCCTCC AAGGCAGACA CCTACACCAC
3061 CAACAACACA ACTTCCCCAA CAAGTGCAGC CTTCACTTCC TGCTGCACCT TCTGCTGACC
3121 AGCCCCAGCA GCAGCCTCGC TCACAGCAGA GCACAGCAGC GTCTGTTCCT ACCCCAACAG
3181 CACCGCTGCT TCCTCCGCAG CCTGCAACTC CACTTTCCCA GCCAGCTGTA AGCATTGAAG
3241 GACAGGTATC AAATCCTCCA TCTACTAGTA GCACAGAAGT GAATTCTCAG GCCATTGCTG
3301 AGAAGCAGCC TTCCCAGGAA GTGAAGATGG AGGCCAAAAT GGAAGTGGAT CAACCAGAAC
3361 CAGCAGATAC TCAGCCGGAG GATATTTCAG AGTCTAAAGT GGAAGACTGT AAAATGGAAT
3421 CTACCGAAAC AGAAGAGAGA AGCACTGAGT TAAAAACTGA AATAAAAGAG GAGGAAGACC
3481 AGCCAAGTAC TTCAGCTACC CAGTCATCTC CGGCTCCAGG ACAGTCAAAG AAAAAGATTT
3541 TCAAACCAGA AGAACTACGA CAGGCACTGA TGCCAACTTT GGAGGCACTT TACCGTCAGG
3601 ATCCAGAATC CCTTCCCTTT CGTCAACCTG TGGACCCTCA GCTTTTAGGA ATCCCTGATT
3661 ACTTTGATAT TGTGAAGAGC CCCATGGATC TTTCTACCAT TAAGAGGAAG TTAGACACTG
3721 GACAGTATCA GGAGCCCTGG CAGTATGTCG ATGATATTTG GCTTATGTTC AATAATGCCT
3781 GGTTATATAA CCGGAAAACA TCACGGGTAT ACAAATACTG CTCCAAGCTC TCTGAGGTCT
3841 TTGAACAAGA AATTGACCCA GTGATGCAAA GCCTTGGATA CTGTTGTGGC AGAAAGTTGG
3901 AGTTCTCTCC ACAGACACTG TGTTGCTACG GCAAACAGTT GTGCACAATA CCTCGTGATG
3961 CCACTTATTA CAGTTACCAG AACAGGTATC ATTTCTGTGA GAAGTGTTTC AATGAGATCC
4021 AAGGGGAGAG CGTTTCTTTG GGGGATGACC CTTCCCAGCC TCAAACTACA ATAAATAAAG
4081 AACAATTTTC CAAGAGAAAA AATGACACAC TGGATCCTGA ACTGTTTGTT GAATGTACAG
4141 AGTGCGGAAG AAAGATGCAT CAGATCTGTG TCCTTCACCA TGAGATCATC TGGCCTGCTG
4201 GATTCGTCTG TGATGGCTGT TTAAAGAAAA GTGCACGAAC TAGGAAAGAA AATAAGTTTT
4261 CTGCTAAAAG GTTGCCATCT ACCAGACTTG GCACCTTTCT AGAGAATCGT GTGAATGACT
4321 TTCTGAGGCG ACAGAATCAC CCTGAGTCAG GAGAGGTCAC TGTTAGAGTA GTTCATGCTT
4381 CTGACAAAAC CGTGGAAGTA AAACCAGGCA TGAAAGCAAG GTTTGTGGAC AGTGGAGAGA
4441 TGGCAGAATC CTTTCCATAC CGAACCAAAG CCCTCTTTGC CTTTGAAGAA ATTGATGGTG
4501 TTGACCTGTG CTTCTTTGGC ATGCATGTTC AAGAGTATGG CTCTGACTGC CCTCCACCCA
4561 ACCAGAGGAG AGTATACATA TCTTACCTCG ATAGTGTTCA TTTCTTCCGT CCTAAATGCT
4621 TGAGGACTGC AGTCTATCAT GAAATCCTAA TTGGATATTT AGAATATGTC AAGAAATTAG
4681 GTTACACAAC AGGGCATATT TGGGCATGTC CACCAAGTGA GGGAGATGAT TATATCTTCC
4741 ATTGCCATCC TCCTGACCAG AAGATACCCA AGCCCAAGCG ACTGCAGGAA TGGTACAAAA
4801 AAATGCTTGA CAAGGCTGTA TCAGAGCGTA TTGTCCATGA CTACAAGGAT ATTTTTAAAC
4861 AAGCTACTGA AGATAGATTA ACAAGTGCAA AGGAATTGCC TTATTTCGAG GGTGATTTCT
4921 GGCCCAATGT TCTGGAAGAA AGCATTAAGG AACTGGAACA GGAGGAAGAA GAGAGAAAAC
4981 GAGAGGAAAA CACCAGCAAT GAAAGCACAG ATGTGACCAA GGGAGACAGC AAAAATGCTA
5041 AAAAGAAGAA TAATAAGAAA ACCAGCAAAA ATAAGAGCAG CCTGAGTAGG GGCAACAAGA
5101 AGAAACCCGG GATGCCCAAT GTATCTAACG ACCTCTCACA GAAACTATAT GCCACCATGG
5161 AGAAGCATAA AGAGGTCTTC TTTGTGATCC GCCTCATTGC TGGCCCTGCT GCCAACTCCC
5221 TGCCTCCCAT TGTTGATCCT GATCCTCTCA TCCCCTGCGA TCTGATGGAT GGTCGGGATG
5281 CGTTTCTCAC GCTGGCAAGG GACAAGCACC TGGAGTTCTC TTCACTCCGA AGAGCCCAGT
5341 GGTCCACCAT GTGCATGCTG GTGGAGCTGC ACACGCAGAG CCAGGACCGC TTTGTCTACA
5401 CCTGCAATGA ATGCAAGCAC CATGTGGAGA CACGCTGGCA CTGTACTGTC TGTGAGGATT
5461 ATGACTTGTG TATCACCTGC TATAACACTA AAAACCATGA CCACAAAATG GAGAAACTAG
5521 GCCTTGGCTT AGATGATGAG AGCAACAACC AGCAGGCTGC AGCCACCCAG AGCCCAGGCG
5581 ATTCTCGCCG CCTGAGTATC CAGCGCTGCA TCCAGTCTCT GGTCCATGCT TGCCAGTGTC
5641 GGAATGCCAA TTGCTCACTG CCATCCTGCC AGAAGATGAA GCGGGTTGTG CAGCATACCA
5701 AGGGTTGCAA ACGGAAAACC AATGGCGGGT GCCCCATCTG CAAGCAGCTC ATTGCCCTCT
5761 GCTGCTACCA TGCCAAGCAC TGCCAGGAGA ACAAATGCCC GGTGCCGTTC TGCCTAAACA
5821 TCAAGCAGAA GCTCCGGCAG CAACAGCTGC AGCACCGACT ACAGCAGGCC CAAATGCTTC
5881 GCAGGAGGAT GGCCAGCATG CAGCGGACTG GTGTGGTTGG GCAGCAACAG GGCCTCCCTT
5941 CCCCCACTCC TGCCACTCCA ACGACACCAA CTGGCCAACA GCCAACCACC CCGCAGACGC
6001 CCCAGCCCAC TTCTCAGCCT CAGCCTACCC CTCCCAATAG CATGCCACCC TACTTGCCCA
6061 GGACTCAAGC TGCTGGCCCT GTGTCCCAGG GTAAGGCAGC AGGCCAGGTG ACCCCTCCAA
6121 CCCCTCCTCA GACTGCTCAG CCACCCCTTC CAGGGCCCCC ACCTGCAGCA GTGGAAATGG
6181 CAATGCAGAT TCAGAGAGCA GCGGAGACGC AGCGCCAGAT GGCCCACGTG CAAATTTTTC
6241 AAAGGCCAAT CCAACACCAG ATGCCCCCGA TGACTCCCAT GGCCCCCATG GGTATGAACC
6301 CACCTCCCAT GACCAGAGGT CCCAGTGGGC ATTTGGAGCC AGGGATGGGA CCGACAGGGA
6361 TGCAGCAACA GCCACCCTGG AGCCAAGGAG GATTGCCTCA GCCCCAGCAA CTACAGTCTG
6421 GGATGCCAAG GCCAGCCATG ATGTCAGTGG CCCAGCATGG TCAACCTTTG AACATGGCTC
6481 CACAACCAGG ATTGGGCCAG GTAGGTATCA GCCCACTCAA ACCAGGCACT GTGTCTCAAC
6541 AAGCCTTACA AAACCTTTTG CGGACTCTCA GGTCTCCCAG CTCTCCCCTG CAGCAGCAAC
6601 AGGTGCTTAG TATCCTTCAC GCCAACCCCC AGCTGTTGGC TGCATTCATC AAGCAGCGGG
6661 CTGCCAAGTA TGCCAACTCT AATCCACAAC CCATCCCTGG GCAGCCTGGC ATGCCCCAGG
6721 GGCAGCCAGG GCTACAGCCA CCTACCATGC CAGGTCAGCA GGGGGTCCAC TCCAATCCAG
6781 CCATGCAGAA CATGAATCCA ATGCAGGCGG GCGTTCAGAG GGCTGGCCTG CCCCAGCAGC
6841 AACCACAGCA GCAACTCCAG CCACCCATGG GAGGGATGAG CCCCCAGGCT CAGCAGATGA
6901 ACATGAACCA CAACACCATG CCTTCACAAT TCCGAGACAT CTTGAGACGA CAGCAAATGA
6961 TGCAACAGCA GCAGCAACAG GGAGCAGGGC CAGGAATAGG CCCTGGAATG GCCAACCATA
7021 ACCAGTTCCA GCAACCCCAA GGAGTTGGCT ACCCACCACA GCAGCAGCAG CGGATGCAGC
7081 ATCACATGCA ACAGATGCAA CAAGGAAATA TGGGACAGAT AGGCCAGCTT CCCCAGGCCT
7141 TGGGAGCAGA GGCAGGTGCC AGTCTACAGG CCTATCAGCA GCGACTCCTT CAGCAACAGA
7201 TGGGGTCCCC TGTTCAGCCC AACCCCATGA GCCCCCAGCA GCATATGCTC CCAAATCAGG
7261 CCCAGTCCCC ACACCTACAA GGCCAGCAGA TCCCTAATTC TCTCTCCAAT CAAGTGCGCT
7321 CTCCCCAGCC TGTCCCTTCT CCACGGCCAC AGTCCCAGCC CCCCCACTCC AGTCCTTCCC
7381 CAAGGATGCA GCCTCAGCCT TCTCCACACC ACGTTTCCCC ACAGACAAGT TCCCCACATC
7441 CTGGACTGGT AGCTGCCCAG GCCAACCCCA TGGAACAAGG GCATTTTGCC AGCCCGGACC
7501 AGAATTCAAT GCTTTCTCAG CTTGCTAGCA ATCCAGGCAT GGCAAACCTC CATGGTGCAA
7561 GCGCCACGGA CCTGGGACTC AGCACCGATA ACTCAGACTT GAATTCAAAC CTCTCACAGA
7621 GTACACTAGA CATACACTAG AGACACCTTG TAGTATTTTG GGAGCAAAAA AATTATTTTC
7681 TCTTAACAAG ACTTTTTGTA CTGAAAACAA TTTTTTTGAA TCTTTCGTAG CCTAAAAGAC
7741 AATTTTCCTT GGAACACATA AGAACTGTGC AGTAGCCGTT TGTGGTTTAA AGCAAACATG
7801 CAAGATGAAC CTGAGGGATG ATAGAATACA AAGAATATAT TTTTGTTATG GCTGGTTACC
7861 ACCAGCCTTT CTTCCCCTTT GTGTGTGTGG TTCAAGTGTG CACTGGGAGG AGGCTGAGGC
7921 CTGTGAAGCC AAACAATATG CTCCTGCCTT GCACCTCCAA TAGGTTTTAT TATTTTTTTT
7981 AAATTAATGA ACATATGTAA TATTAATAGT TATTATTTAC TGGTGCAGAT GGTTGACATT
8041 TTTCCCTATT TTCCTCACTT TATGGAAGAG TTAAAACATT TCTAAACCAG AGGACAAAAG
8101 GGGTTAATGT TACTTTAAAA TTACATTCTA TATATATATA AATATATATA AATATATATT
8161 AAAATACCAG TTTTTTTTCT CTGGGTGCAA AGATGTTCAT TCTTTTAAAA AATGTTTAAA
8221 AAAAAAAAAA AACTGCCTTT CTTCCCCTCA AGTCAACTTT TGTGCTCCAG AAAATTTTCT
8281 ATTCTGTAAG TCTGAGCGTA AAACTTCAAG TATTAAAATA ATTTGTACAT GTAGAGAGAA
8341 AAATGACTTT TTCAAAAATA TACAGGGGCA GCTGCCAAAT TGATGTATTA TATATTGTGG
8401 TTTCTGTTTC TTGAAAGAAT TTTTTTCGTT ATTTTTACAT CTAACAAAGT AAAAAAATTA
8461 AAAAGAGGGT AAGAAACGAT TCCGGTGGGA TGATTTTAAC ATGCAAAATG TCCCTGGGGG
8521 TTTCTTCTTT GCTTGCTTTC TTCCTCCTTA CCCTACCCCC CACTCACACA CACACACACA
8581 CACACACACA CACACACACA CACACACTTT CTATAAAACT TGAAAATAGC AAAAACCCTC
8641 AACTGTTGTA AATCATGCAA TTAAAGTTGA TTACTTATAA ATATGAACTT TGGATCACTG
8701 TATAGACTGT TAAATTTGAT TTCTTATTAC CTATTGTTAA ATAAACTGTG TGAGACAGAC
8761 A

In certain embodiments, a mutation of the disclosure may occur in a sequence encoding the p300 HAT, including the amino acid sequence of the p300 protein (below, corresponding to GenBank Accession No. NP_001420.2, defined as Homo sapiens E1A-binding protein, 300 kD; E1A-associated protein p300; p300 HAT; and identified as SEQ ID NO: 20).

1 MAENVVEPGP PSAKRPKLSS PALSASASDG TDFGSLFDLE HDLPDELINS TELGLTNGGD
61 INQLQTSLGM VQDAASKHKQ LSELLRSGSS PNLNMGVGGP GQVMASQAQQ SSPGLGLINS
121 MVKSPMTQAG LTSPNMGMGT SGPNQGPTQS TGMMNSPVNQ PAMGMNTGMN AGMNPGMLAA
181 GNGQGIMPNQ VMNGSIGAGR GRQNMQYPNP GMGSAGNLLT EPLQQGSPQM GGQTGLRGPQ
241 PLKMGMMNNP NPYGSPYTQN PGQQIGASGL GLQIQTKTVL SNNLSPFAMD KKAVPGGGMP
301 NMGQQPAPQV QQPGLVTPVA QGMGSGAHTA DPEKRKLIQQ QLVLLLHAHK CQRREQANGE
361 VRQCNLPHCR TMKNVLNHMT HCQSGKSCQV AHCASSRQII SHWKNCTRHD CPVCLPLKNA
421 GDKRNQQPIL TGAPVGLGNP SSLGVGQQSA PNLSTVSQID PSSIERAYAA LGLPYQVNQM
481 PTQPQVQAKN QQNQQPGQSP QGMRPMSNMS ASPMGVNGGV GVQTPSLLSD SMLHSAINSQ
541 NPMMSENASV PSLGPMPTAA QPSTTGIRKQ WHEDITQDLR NHLVHKLVQA IFPTPDPAAL
601 KDRRMENLVA YARKVEGDMY ESANNRAEYY HLLAEKIYKI QKELEEKRRT RLQKQNMLPN
661 AAGMVPVSMN PGPNMGQPQP GMTSNGPLPD PSMIRGSVPN QMMPRITPQS GLNQFGQMSM
721 AQPPIVPRQT PPLQHHGQLA QPGALNPPMG YGPRMQQPSN QGQFLPQTQF PSQGMNVTNI
781 PLAPSSGQAP VSQAQMSSSS CPVNSPIMPP GSQGSHIHCP QLPQPALHQN SPSPVPSRTP
841 TPHHTPPSIG AQQPPATTIP APVPTPPAMP PGPQSQALHP PPRQTPTPPT TQLPQQVQPS
901 LPAAPSADQP QQQPRSQQST AASVPTPTAP LLPPQPATPL SQPAVSIEGQ VSNPPSTSST
961 EVNSQAIAEK QPSQEVKMEA KMEVDQPEPA DTQPEDISES KVEDCKMEST ETEERSTELK
1021 TEIKEEEDQP STSATQSSPA PGQSKKKIFK PEELRQALMP TLEALYRQDP ESLPFRQPVD
1081 PQLLGIPDYF DIVKSPMDLS TIKRKLDTGQ YQEPWQYVDD IWLMFNNAWL YNRKTSRVYK
1141 YCSKLSEVFE QEIDPVMQSL GYCCGRKLEF SPQTLCCYGK QLCTIPRDAT YYSYQNRYHF
1201 CEKCFNEIQG ESVSLGDDPS QPQTTINKEQ FSKRKNDTLD PELFVECTEC GRKMHQICVL
1261 HHEIIWPAGF VCDGCLKKSA RTRKENKFSA KRLPSTRLGT FLENRVNDFL RRQNHPESGE
1321 VTVRVVHASD KTVEVKPGMK ARFVDSGEMA ESFPYRTKAL FAFEEIDGVD LCFFGMHVQE
1381 YGSDCPPPNQ RRVYISYLDS VHFFRPKCLR TAVYHEILIG YLEYVKKLGY TTGHIWACPP
1441 SEGDDYIFHC HPPDQKIPKP KRLQEWYKKM LDKAVSERIV HDYKDIFKQA TEDRLTSAKE
1501 LPYFEGDFWP NVLEESIKEL EQEEEERKRE ENTSNESTDV TKGDSKNAKK KNNKKTSKNK
1561 SSLSRGNKKK PGMPNVSNDL SQKLYATMEK HKEVFFVIRL IAGPAANSLP PIVDPDPLIP
1621 CDLMDGRDAF LTLARDKHLE FSSLRRAQWS TMCMLVELHT QSQDRFVYTC NECKHHVETR
1681 WHCTVCEDYD LCITCYNTKN HDHKMEKLGL GLDDESNNQQ AAATQSPGDS RRLSIQRCIQ
1741 SLVHACQCRN ANCSLPSCQK MKRVVQHTKG CKRKTNGGCP ICKQLIALCC YHAKHCQENK
1801 CPVPFCLNIK QKLRQQQLQH RLQQAQMLRR RMASMQRTGV VGQQQGLPSP TPATPTTPTG
1861 QQPTTPQTPQ PTSQPQPTPP NSMPPYLPRT QAAGPVSQGK AAGQVTPPTP PQTAQPPLPG
1921 PPPAAVEMAM QIQRAAETQR QMAHVQIFQR PIQHQMPPMT PMAPMGMNPP PMTRGPSGHL
1981 EPGMGPTGMQ QQPPWSQGGL PQPQQLQSGM PRPAMMSVAQ HGQPLNMAPQ PGLGQVGISP
2041 LKPGTVSQQA LQNLLRTLRS PSSPLQQQQV LSILHANPQL LAAFIKQRAA KYANSNPQPI
2101 PGQPGMPQGQ PGLQPPTMPG QQGVHSNPAM QNMNPMQAGV QRAGLPQQQP QQQLQPPMGG
2161 MSPQAQQMNM NHNTMPSQFR DILRRQQMMQ QQQQQGAGPG IGPGMANHNQ FQQPQGVGYP
2221 PQQQQRMQHH MQQMQQGNMG QIGQLPQALG AEAGASLQAY QQRLLQQQMG SPVQPNPMSP
2281 QQHMLPNQAQ SPHLQGQQIP NSLSNQVRSP QPVPSPRPQS QPPHSSPSPR MQPQPSPHHV
2341 SPQTSSPHPG LVAAQANPME QGHFASPDQN SMLSQLASNP GMANLHGASA TDLGLSTDNS
2401 DLNSNLSQST LDIH

In certain embodiments, a mutation of the disclosure may occur in a sequence encoding the CREB Binding Protein (CREBBP) HAT, including the nucleotide sequence encoding CREBBP (below, corresponding to GenBank Accession No. NM_004380, defined as Homo sapiens CREB binding protein (CREBBP), transcript variant 1, mRNA; and identified as SEQ ID NO: 23).

1 CTGCGGGGCG CTGTTGCTGT GGCTGAGATT TGGCCGCCGC CTCCCCCACC CGGCCTGCGC
61 CCTCCCTCTC CCTCGGCGCC CGCCCGCCCG CTCGCGGCCC GCGCTCGCTC CTCTCCCTCG
121 CAGCCGGCAG GGCCCCCGAC CCCCGTCCGG GCCCTCGCCG GCCCGGCCGC CCGTGCCCGG
181 GGCTGTTTTC GCGAGCAGGT GAAAATGGCT GAGAACTTGC TGGACGGACC GCCCAACCCC
241 AAAAGAGCCA AACTCAGCTC GCCCGGTTTC TCGGCGAATG ACAGCACAGA TTTTGGATCA
301 TTGTTTGACT TGGAAAATGA TCTTCCTGAT GAGCTGATAC CCAATGGAGG AGAATTAGGC
361 CTTTTAAACA GTGGGAACCT TGTTCCAGAT GCTGCTTCCA AACATAAACA ACTGTCGGAG
421 CTTCTACGAG GAGGCAGCGG CTCTAGTATC AACCCAGGAA TAGGAAATGT GAGCGCCAGC
481 AGCCCCGTGC AGCAGGGCCT GGGTGGCCAG GCTCAAGGGC AGCCGAACAG TGCTAACATG
541 GCCAGCCTCA GTGCCATGGG CAAGAGCCCT CTGAGCCAGG GAGATTCTTC AGCCCCCAGC
601 CTGCCTAAAC AGGCAGCCAG CACCTCTGGG CCCACCCCCG CTGCCTCCCA AGCACTGAAT
661 CCGCAAGCAC AAAAGCAAGT GGGGCTGGCG ACTAGCAGCC CTGCCACGTC ACAGACTGGA
721 CCTGGTATCT GCATGAATGC TAACTTTAAC CAGACCCACC CAGGCCTCCT CAATAGTAAC
781 TCTGGCCATA GCTTAATTAA TCAGGCTTCA CAAGGGCAGG CGCAAGTCAT GAATGGATCT
841 CTTGGGGCTG CTGGCAGAGG AAGGGGAGCT GGAATGCCGT ACCCTACTCC AGCCATGCAG
901 GGCGCCTCGA GCAGCGTGCT GGCTGAGACC CTAACGCAGG TTTCCCCGCA AATGACTGGT
961 CACGCGGGAC TGAACACCGC ACAGGCAGGA GGCATGGCCA AGATGGGAAT AACTGGGAAC
1021 ACAAGTCCAT TTGGACAGCC CTTTAGTCAA GCTGGAGGGC AGCCAATGGG AGCCAATGGG
1081 GTGAACCCCC AGTTAGCCAG CAAACAGAGC ATGGTCAACA GTTTGCCCAC CTTCCCTACA
1141 GATATCAAGA ATACTTCAGT CACCAACGTG CCAAATATGT CTCAGATGCA AACATCAGTG
1201 GGAATTGTAC CCACACAAGC AATTGCAACA GGCCCCACTG CAGATCCTGA AAAACGCAAA
1261 CTGATACAGC AGCAGCTGGT TCTACTGCTT CATGCTCATA AGTGTCAGAG ACGAGAGCAA
1321 GCAAACGGAG AGGTTCGGGC CTGCTCGCTC CCGCATTGTC GAACCATGAA AAACGTTTTG
1381 AATCACATGA CGCATTGTCA GGCTGGGAAA GCCTGCCAAG TTGCCCATTG TGCATCTTCA
1441 CGACAAATCA TCTCTCATTG GAAGAACTGC ACACGACATG ACTGTCCTGT TTGCCTCCCT
1501 TTGAAAAATG CCAGTGACAA GCGAAACCAA CAAACCATCC TGGGGTCTCC AGCTAGTGGA
1561 ATTCAAAACA CAATTGGTTC TGTTGGCACA GGGCAACAGA ATGCCACTTC TTTAAGTAAC
1621 CCAAATCCCA TAGACCCCAG CTCCATGCAG CGAGCCTATG CTGCTCTCGG ACTCCCCTAC
1681 ATGAACCAGC CCCAGACGCA GCTGCAGCCT CAGGTTCCTG GCCAGCAACC AGCACAGCCT
1741 CAAACCCACC AGCAGATGAG GACTCTCAAC CCCCTGGGAA ATAATCCAAT GAACATTCCA
1801 GCAGGAGGAA TAACAACAGA TCAGCAGCCC CCAAACTTGA TTTCAGAATC AGCTCTTCCG
1861 ACTTCCCTGG GGGCCACAAA CCCACTGATG AACGATGGCT CCAACTCTGG TAACATTGGA
1921 ACCCTCAGCA CTATACCAAC AGCAGCTCCT CCTTCTAGCA CCGGTGTAAG GAAAGGCTGG
1981 CACGAACATG TCACTCAGGA CCTGCGGAGC CATCTAGTGC ATAAACTCGT CCAAGCCATC
2041 TTCCCAACAC CTGATCCCGC AGCTCTAAAG GATCGCCGCA TGGAAAACCT GGTAGCCTAT
2101 GCTAAGAAAG TGGAAGGGGA CATGTACGAG TCTGCCAACA GCAGGGATGA ATATTATCAC
2161 TTATTAGCAG AGAAAATCTA CAAGATACAA AAAGAACTAG AAGAAAAACG GAGGTCGCGT
2221 TTACATAAAC AAGGCATCTT GGGGAACCAG CCAGCCTTAC CAGCCCCGGG GGCTCAGCCC
2281 CCTGTGATTC CACAGGCACA ACCTGTGAGA CCTCCAAATG GACCCCTGTC CCTGCCAGTG
2341 AATCGCATGC AAGTTTCTCA AGGGATGAAT TCATTTAACC CCATGTCCTT GGGGAACGTC
2401 CAGTTGCCAC AAGCACCCAT GGGACCTCGT GCAGCCTCCC CAATGAACCA CTCTGTCCAG
2461 ATGAACAGCA TGGGCTCAGT GCCAGGGATG GCCATTTCTC CTTCCCGAAT GCCTCAGCCT
2521 CCGAACATGA TGGGTGCACA CACCAACAAC ATGATGGCCC AGGCGCCCGC TCAGAGCCAG
2581 TTTCTGCCAC AGAACCAGTT CCCGTCATCC AGCGGGGCGA TGAGTGTGGG CATGGGGCAG
2641 CCGCCAGCCC AAACAGGCGT GTCACAGGGA CAGGTGCCTG GTGCTGCTCT TCCTAACCCT
2701 CTCAACATGC TGGGGCCTCA GGCCAGCCAG CTACCTTGCC CTCCAGTGAC ACAGTCACCA
2761 CTGCACCCAA CACCGCCTCC TGCTTCCACG GCTGCTGGCA TGCCATCTCT CCAGCACACG
2821 ACACCACCTG GGATGACTCC TCCCCAGCCA GCAGCTCCCA CTCAGCCATC AACTCCTGTG
2881 TCGTCTTCCG GGCAGACTCC CACCCCGACT CCTGGCTCAG TGCCCAGTGC TACCCAAACC
2941 CAGAGCACCC CTACAGTCCA GGCAGCAGCC CAGGCCCAGG TGACCCCGCA GCCTCAAACC
3001 CCAGTTCAGC CCCCGTCTGT GGCTACCCCT CAGTCATCGC AGCAACAGCC GACGCCTGTG
3061 CACGCCCAGC CTCCTGGCAC ACCGCTTTCC CAGGCAGCAG CCAGCATTGA TAACAGAGTC
3121 CCTACCCCCT CCTCGGTGGC CAGCGCAGAA ACCAATTCCC AGCAGCCAGG ACCTGACGTA
3181 CCTGTGCTGG AAATGAAGAC GGAGACCCAA GCAGAGGACA CTGAGCCCGA TCCTGGTGAA
3241 TCCAAAGGGG AGCCCAGGTC TGAGATGATG GAGGAGGATT TGCAAGGAGC TTCCCAAGTT
3301 AAAGAAGAAA CAGACATAGC AGAGCAGAAA TCAGAACCAA TGGAAGTGGA TGAAAAGAAA
3361 CCTGAAGTGA AAGTAGAAGT TAAAGAGGAA GAAGAGAGTA GCAGTAACGG CACAGCCTCT
3421 CAGTCAACAT CTCCTTCGCA GCCGCGCAAA AAAATCTTTA AACCAGAGGA GTTACGCCAG
3481 GCCCTCATGC CAACCCTAGA AGCACTGTAT CGACAGGACC CAGAGTCATT ACCTTTCCGG
3541 CAGCCTGTAG ATCCCCAGCT CCTCGGAATT CCAGACTATT TTGACATCGT AAAGAATCCC
3601 ATGGACCTCT CCACCATCAA GCGGAAGCTG GACACAGGGC AATACCAAGA GCCCTGGCAG
3661 TACGTGGACG ACGTCTGGCT CATGTTCAAC AATGCCTGGC TCTATAATCG CAAGACATCC
3721 CGAGTCTATA AGTTTTGCAG TAAGCTTGCA GAGGTCTTTG AGCAGGAAAT TGACCCTGTC
3781 ATGCAGTCCC TTGGATATTG CTGTGGACGC AAGTATGAGT TTTCCCCACA GACTTTGTGC
3841 TGCTATGGGA AGCAGCTGTG TACCATTCCT CGCGATGCTG CCTACTACAG CTATCAGAAT
3901 AGGTATCATT TCTGTGAGAA GTGTTTCACA GAGATCCAGG GCGAGAATGT GACCCTGGGT
3961 GACGACCCTT CACAGCCCCA GACGACAATT TCAAAGGATC AGTTTGAAAA GAAGAAAAAT
4021 GATACCTTAG ACCCCGAACC TTTCGTTGAT TGCAAGGAGT GTGGCCGGAA GATGCATCAG
4081 ATTTGCGTTC TGCACTATGA CATCATTTGG CCTTCAGGTT TTGTGTGCGA CAACTGCTTG
4141 AAGAAAACTG GCAGACCTCG AAAAGAAAAC AAATTCAGTG CTAAGAGGCT GCAGACCACA
4201 AGACTGGGAA ACCACTTGGA AGACCGAGTG AACAAATTTT TGCGGCGCCA GAATCACCCT
4261 GAAGCCGGGG AGGTTTTTGT CCGAGTGGTG GCCAGCTCAG ACAAGACGGT GGAGGTCAAG
4321 CCCGGGATGA AGTCACGGTT TGTGGATTCT GGGGAAATGT CTGAATCTTT CCCATATCGA
4381 ACCAAAGCTC TGTTTGCTTT TGAGGAAATT GACGGCGTGG ATGTCTGCTT TTTTGGAATG
4441 CACGTCCAAG AATACGGCTC TGATTGCCCC CCTCCAAACA CGAGGCGTGT GTACATTTCT
4501 TATCTGGATA GTATTCATTT CTTCCGGCCA CGTTGCCTCC GCACAGCCGT TTACCATGAG
4561 ATCCTTATTG GATATTTAGA GTATGTGAAG AAATTAGGGT ATGTGACAGG GCACATCTGG
4621 GCCTGTCCTC CAAGTGAAGG AGATGATTAC ATCTTCCATT GCCACCCACC TGATCAAAAA
4681 ATACCCAAGC CAAAACGACT GCAGGAGTGG TACAAAAAGA TGCTGGACAA GGCGTTTGCA
4741 GAGCGGATCA TCCATGACTA CAAGGATATT TTCAAACAAG CAACTGAAGA CAGGCTCACC
4801 AGTGCCAAGG AACTGCCCTA TTTTGAAGGT GATTTCTGGC CCAATGTGTT AGAAGAGAGC
4861 AGGAAAAAGG TAGAACAAGA AGAAGAGGAG AGGAAAAAGG AAGAGAGCAC TGCAGCCAGT
4921 GAAACCACTG AGGGCAGTCA GGGCGACAGC AAGAATGCCA AGAAGAAGAA CAACAAGAAA
4981 ACCAACAAGA ACAAAAGCAG CATCAGCCGC GCCAACAAGA AGAAGCCCAG CATGCCCAAC
5041 GTGTCCAATG ACCTGTCCCA GAAGCTGTAT GCCACCATGG AGAAGCACAA GGAGGTCTTC
5101 TTCGTGATCC ACCTGCACGC TGGGCCTGTC ATCAACACCC TGCCCCCCAT CGTCGACCCC
5161 GACCCCCTGC TCAGCTGTGA CCTCATGGAT GGGCGCGACG CCTTCCTCAC CCTCGCCAGA
5221 GACAAGCACT GGGAGTTCTC CTCCTTGCGC CGCTCCAAGT GGTCCACGCT CTGCATGCTG
5281 GTGGAGCTGC ACACCCAGGG CCAGGACCGC TTTGTCTACA CCTGCAACGA GTGCAAGCAC
5341 CACGTGGAGA CGCGCTGGCA CTGCACTGTG TGCGAGGACT ACGACCTCTG CATCAACTGC
5401 TATAACACGA AGAGCCATGC CCATAAGATG GTGAAGTGGG GGCTGGGCCT GGATGACGAG
5461 GGCAGCAGCC AGGGCGAGCC ACAGTCAAAG AGCCCCCAGG AGTCACGCCG GCTGAGCATC
5521 CAGCGCTGCA TCCAGTCGCT GGTGCACGCG TGCCAGTGCC GCAACGCCAA CTGCTCGCTG
5581 CCATCCTGCC AGAAGATGAA GCGGGTGGTG CAGCACACCA AGGGCTGCAA ACGCAAGACC
5641 AACGGGGGCT GCCCGGTGTG CAAGCAGCTC ATCGCCCTCT GCTGCTACCA CGCCAAGCAC
5701 TGCCAAGAAA ACAAATGCCC CGTGCCCTTC TGCCTCAACA TCAAACACAA GCTCCGCCAG
5761 CAGCAGATCC AGCACCGCCT GCAGCAGGCC CAGCTCATGC GCCGGCGGAT GGCCACCATG
5821 AACACCCGCA ACGTGCCTCA GCAGAGTCTG CCTTCTCCTA CCTCAGCACC GCCCGGGACC
5881 CCCACACAGC AGCCCAGCAC ACCCCAGACG CCGCAGCCCC CTGCCCAGCC CCAACCCTCA
5941 CCCGTGAGCA TGTCACCAGC TGGCTTCCCC AGCGTGGCCC GGACTCAGCC CCCCACCACG
6001 GTGTCCACAG GGAAGCCTAC CAGCCAGGTG CCGGCCCCCC CACCCCCGGC CCAGCCCCCT
6061 CCTGCAGCGG TGGAAGCGGC TCGGCAGATC GAGCGTGAGG CCCAGCAGCA GCAGCACCTG
6121 TACCGGGTGA ACATCAACAA CAGCATGCCC CCAGGACGCA CGGGCATGGG GACCCCGGGG
6181 AGCCAGATGG CCCCCGTGAG CCTGAATGTG CCCCGACCCA ACCAGGTGAG CGGGCCCGTC
6241 ATGCCCAGCA TGCCTCCCGG GCAGTGGCAG CAGGCGCCCC TTCCCCAGCA GCAGCCCATG
6301 CCAGGCTTGC CCAGGCCTGT GATATCCATG CAGGCCCAGG CGGCCGTGGC TGGGCCCCGG
6361 ATGCCCAGCG TGCAGCCACC CAGGAGCATC TCACCCAGCG CTCTGCAAGA CCTGCTGCGG
6421 ACCCTGAAGT CGCCCAGCTC CCCTCAGCAG CAACAGCAGG TGCTGAACAT TCTCAAATCA
6481 AACCCGCAGC TAATGGCAGC TTTCATCAAA CAGCGCACAG CCAAGTACGT GGCCAATCAG
6541 CCCGGCATGC AGCCCCAGCC TGGCCTCCAG TCCCAGCCCG GCATGCAACC CCAGCCTGGC
6601 ATGCACCAGC AGCCCAGCCT GCAGAACCTG AATGCCATGC AGGCTGGCGT GCCGCGGCCC
6661 GGTGTGCCTC CACAGCAGCA GGCGATGGGA GGCCTGAACC CCCAGGGCCA GGCCTTGAAC
6721 ATCATGAACC CAGGACACAA CCCCAACATG GCGAGTATGA ATCCACAGTA CCGAGAAATG
6781 TTACGGAGGC AGCTGCTGCA GCAGCAGCAG CAACAGCAGC AGCAACAACA GCAGCAACAG
6841 CAGCAGCAGC AAGGGAGTGC CGGCATGGCT GGGGGCATGG CGGGGCACGG CCAGTTCCAG
6901 CAGCCTCAAG GACCCGGAGG CTACCCACCG GCCATGCAGC AGCAGCAGCG CATGCAGCAG
6961 CATCTCCCCC TCCAGGGCAG CTCCATGGGC CAGATGGCGG CTCAGATGGG ACAGCTTGGC
7021 CAGATGGGGC AGCCGGGGCT GGGGGCAGAC AGCACCCCCA ACATCCAGCA AGCCCTGCAG
7081 CAGCGGATTC TGCAGCAACA GCAGATGAAG CAGCAGATTG GGTCCCCAGG CCAGCCGAAC
7141 CCCATGAGCC CCCAGCAACA CATGCTCTCA GGACAGCCAC AGGCCTCGCA TCTCCCTGGC
7201 CAGCAGATCG CCACGTCCCT TAGTAACCAG GTGCGGTCTC CAGCCCCTGT CCAGTCTCCA
7261 CGGCCCCAGT CCCAGCCTCC ACATTCCAGC CCGTCACCAC GGATACAGCC CCAGCCTTCG
7321 CCACACCACG TCTCACCCCA GACTGGTTCC CCCCACCCCG GACTCGCAGT CACCATGGCC
7381 AGCTCCATAG ATCAGGGACA CTTGGGGAAC CCCGAACAGA GTGCAATGCT CCCCCAGCTG
7441 AACACCCCCA GCAGGAGTGC GCTGTCCAGC GAACTGTCCC TGGTCGGGGA CACCACGGGG
7501 GACACGCTAG AGAAGTTTGT GGAGGGCTTG TAGCATTGTG AGAGCATCAC CTTTTCCCTT
7561 TCATGTTCTT GGACCTTTTG TACTGAAAAT CCAGGCATCT AGGTTCTTTT TATTCCTAGA
7621 TGGAACTGCG ACTTCCGAGC CATGGAAGGG TGGATTGATG TTTAAAGAAA CAATACAAAG
7681 AATATATTTT TTTGTTAAAA ACCAGTTGAT TTAAATATCT GGTCTCTCTC TTTGGTTTTT
7741 TTTTGGCGGG GGGGTGGGGG GGGTTCTTTT TTTTCCGTTT TGTTTTTGTT TGGGGGGAGG
7801 GGGGTTTTGT TTGGATTCTT TTTGTCGTCA TTGCTGGTGA CTCATGCCTT TTTTTAACGG
7861 GAAAAACAAG TTCATTATAT TCATATTTTT TATTTGTATT TTCAAGACTT TAAACATTTA
7921 TGTTTAAAAG TAAGAAGAAA AATAATATTC AGAACTGATT CCTGAAATAA TGCAAGCTTA
7981 TAATGTATCC CGATAACTTT GTGATGTTTC GGGAAGATTT TTTTCTATAG TGAACTCTGT
8041 GGGCGTCTCC CAGTATTACC CTGGATGATA GGAATTGACT CCGGCGTGCA CACACGTACA
8101 CACCCACACA CATCTATCTA TACATAATGG CTGAAGCCAA ACTTGTCTTG CAGATGTAGA
8161 AATTGTTGCT TTGTTTCTCT GATAAAACTG GTTTTAGACA AAAAATAGGG ATGATCACTC
8221 TTAGACCATG CTAATGTTAC TAGAGAAGAA GCCTTCTTTT CTTTCTTCTA TGTGAAACTT
8281 GAAATGAGGA AAAGCAATTC TAGTGTAAAT CATGCAAGCG CTCTAATTCC TATAAATACG
8341 AAACTCGAGA AGATTCAATC ACTGTATAGA ATGGTAAAAT ACCAACTCAT TTCTTATATC
8401 ATATTGTTAA ATAAACTGTG TGCAACAGAC AAAAAGGGTG GTCCTTCTTG AATTCATGTA
8461 CATGGTATTA ACACTTAGTG TTCGGGGTTT TTTGTTATGA AAATGCTGTT TTCAACATTG
8521 TATTTGGACT ATGCATGTGT TTTTTCCCCA TTGTATATAA AGTACCGCTT AAAATTGATA
8581 TAAATTACTG AGGTTTTTAA CATGTATTCT GTTCTTTAAG ATCCCTGTAA GAATGTTTAA
8641 GGTTTTTATT TATTTATATA TATTTTTTGA GTCTGTTCTT TGTAAGACAT GGTTCTGGTT
8701 GTTCGCTCAT AGCGGAGAGG CTGGGGCTGC GGTTGTGGTT GTGGCGGCGT GGGTGGTGGC
8761 TGGGAACTGT GGCCCAGGCT TAGCGGCCGC CCGGAGGCTT TTCTTCCCGG AGACTGAGGT
8821 GGGCGACTGA GGTGGGCGGC TCAGCGTTGG CCCCACACAT TCGAGGCTCA CAGGTGATTG
8881 TCGCTCACAC AGTTAGGGTC GTCAGTTGGT CTGAAACTGC ATTTGGCCCA CTCCTCCATC
8941 CTCCCTGTCC GTCGTAGCTG CCACCCCCAG AGGCGGCGCT TCTTCCCGTG TTCAGGCGGC
9001 TCCCCCCCCC CGTACACGAC TCCCAGAATC TGAGGCAGAG AGTGCTCCAG GCTCGCGAGG
9061 TGCTTTCTGA CTTCCCCCCA AATCCTGCCG CTGCCGCGCA GCATGTCCCG TGTGGCGTTT
9121 GAGGAAATGC TGAGGGACAG ACACCTTGGA GCACCAGCTC CGGTCCCTGT TACAGTGAGA
9181 AAGGTCCCCC ACTTCGGGGG ATACTTGCAC TTAGCCACAT GGTCCTGCCT CCCTTGGAGT
9241 CCAGTTCCAG GCTCCCTTAC TGAGTGGGTG AGACAAGTTC ACAAAAACCG TAAAACTGAG
9301 AGGAGGACCA TGGGCAGGGG AGCTGAAGTT CATCCCCTAA GTCTACCACC CCCAGCACCC
9361 AGAGAACCCA CTTTATCCCT AGTCCCCCAA CAAAGGCTGG TCTAGGTGGG GGTGATGGTA
9421 ATTTTAGAAA TCACGCCCCA AATAGCTTCC GTTTGGGCCC TTACATTCAC AGATAGGTTT
9481 TAAATAGCTG AATACTTGGT TTGGGAATCT GAATTCGAGG AACCTTTCTA AGAAGTTGGA
9541 AAGGTCCGAT CTAGTTTTAG CACAGAGCTT TGAACCTTGA GTTATAAAAT GCAGAATAAT
9601 TCAAGTAAAA ATAAGACCAC CATCTGGCAC CCCTGACCAG CCCCCATTCA CCCCATCCCA
9661 GGAGGGGAAG CACAGGCCGG GCCTCCGGTG GAGATTGCTG CCACTGCTCG GCCTGCTGGG
9721 TTCTTAACCT CCAGTGTCCT CTTCATCTTT TCCACCCGTA GGGAAACCTT GAGCCATGTG
9781 TTCAAACAAG AAGTGGGGCT AGAGCCCGAG AGCAGCAGCT CTAAGCCCAC ACTCAGAAAG
9841 TGGCGCCCTC CTGGTTGTGC AGCCTTTTAA TGTGGGCAGT GGAGGGGCCT CTGTTTCAGG
9901 TTATCCTGGA ATTCAAAACG TTATGTACCA ACCTCATCCT CTTTGGAGTC TGCATCCTGT
9961 GCAACCGTCT TGGGCAATCC AGATGTCGAA GGATGTGACC GAGAGCATGG TCTGTGGATG
10021 CTAACCCTAA GTTTGTCGTA AGGAAATTTC TGTAAGAAAC CTGGAAAGCC CCAACGCTGT
10081 GTCTCATGCT GTATACTTAA GAGGAGAAGA AAAAGTCCTA TATTTGTGAT CAAAAAGAGG
10141 AAACTTGAAA TGTGATGGTG TTTATAATAA AAGATGGTAA AACTACTTGG ATTCAAA

In certain embodiments, a mutation of the disclosure may occur in a sequence encoding the CREB Binding Protein (CREBBP) HAT, including the amino acid sequence encoding CREBBP (below, corresponding to GenBank Accession No. NP_004371, defined as Homo sapiens CREB-binding protein isoform a; and identified as SEQ ID NO: 24).

1 MAENLLDGPP NPKRAKLSSP GFSANDSTDF GSLFDLENDL PDELIPNGGE LGLLNSGNLV
61 PDAASKHKQL SELLRGGSGS SINPGIGNVS ASSPVQQGLG GQAQGQPNSA NMASLSAMGK
121 SPLSQGDSSA PSLPKQAAST SGPTPAASQA LNPQAQKQVG LATSSPATSQ TGPGICMNAN
181 FNQTHPGLLN SNSGHSLINQ ASQGQAQVMN GSLGAAGRGR GAGMPYPTPA MQGASSSVLA
241 ETLTQVSPQM TGHAGLNTAQ AGGMAKMGIT GNTSPFGQPF SQAGGQPMGA TGVNPQLASK
301 QSMVNSLPTF PTDIKNTSVT NVPNMSQMQT SVGIVPTQAI ATGPTADPEK RKLIQQQLVL
361 LLHAHKCQRR EQANGEVRAC SLPHCRTMKN VLNHMTHCQA GKACQVAHCA SSRQIISHWK
421 NCTRHDCPVC LPLKNASDKR NQQTILGSPA SGIQNTIGSV GTGQQNATSL SNPNPIDPSS
481 MQRAYAALGL PYMNQPQTQL QPQVPGQQPA QPQTHQQMRT LNPLGNNPMN IPAGGITTDQ
541 QPPNLISESA LPTSLGATNP LMNDGSNSGN IGTLSTIPTA APPSSTGVRK GWHEHVTQDL
601 RSHLVHKLVQ AIFPTPDPAA LKDRRMENLV AYAKKVEGDM YESANSRDEY YHLLAEKIYK
661 IQKELEEKRR SRLHKQGILG NQPALPAPGA QPPVIPQAQP VRPPNGPLSL PVNRMQVSQG
721 MNSFNPMSLG NVQLPQAPMG PRAASPMNHS VQMNSMGSVP GMAISPSRMP QPPNMMGAHT
781 NNMMAQAPAQ SQFLPQNQFP SSSGAMSVGM GQPPAQTGVS QGQVPGAALP NPLNMLGPQA
841 SQLPCPPVTQ SPLHPTPPPA STAAGMPSLQ HTTPPGMTPP QPAAPTQPST PVSSSGQTPT
901 PTPGSVPSAT QTQSTPTVQA AAQAQVTPQP QTPVQPPSVA TPQSSQQQPT PVHAQPPGTP
961 LSQAAASIDN RVPTPSSVAS AETNSQQPGP DVPVLEMKTE TQAEDTEPDP GESKGEPRSE
1021 MMEEDLQGAS QVKEETDIAE QKSEPMEVDE KKPEVKVEVK EEEESSSNGT ASQSTSPSQP
1081 RKKIFKPEEL RQALMPTLEA LYRQDPESLP FRQPVDPQLL GIPDYFDIVK NPMDLSTIKR
1141 KLDTGQYQEP WQYVDDVWLM FNNAWLYNRK TSRVYKFCSK LAEVFEQEID PVMQSLGYCC
1201 GRKYEFSPQT LCCYGKQLCT IPRDAAYYSY QNRYHFCEKC FTEIQGENVT LGDDPSQPQT
1261 TISKDQFEKK KNDTLDPEPF VDCKECGRKM HQICVLHYDI IWPSGFVCDN CLKKTGRPRK
1321 ENKFSAKRLQ TTRLGNHLED RVNKFLRRQN HPEAGEVFVR VVASSDKTVE VKPGMKSRFV
1381 DSGEMSESFP YRTKALFAFE EIDGVDVCFF GMHVQEYGSD CPPPNTRRVY ISYLDSIHFF
1441 RPRCLRTAVY HEILIGYLEY VKKLGYVTGH IWACPPSEGD DYIFHCHPPD QKIPKPKRLQ
1501 EWYKKMLDKA FAERIIHDYK DIFKQATEDR LTSAKELPYF EGDFWPNVLE ESIKELEQEE
1561 EERKKEESTA ASETTEGSQG DSKNAKKKNN KKTNKNKSSI SRANKKKPSM PNVSNDLSQK
1621 LYATMEKHKE VFFVIHLHAG PVINTLPPIV DPDPLLSCDL MDGRDAFLTL ARDKHWEFSS
1681 LRRSKWSTLC MLVELHTQGQ DRFVYTCNEC KHHVETRWHC TVCEDYDLCI NCYNTKSHAH
1741 KMVKWGLGLD DEGSSQGEPQ SKSPQESRRL SIQRCIQSLV HACQCRNANC SLPSCQKMKR
1801 VVQHTKGCKR KTNGGCPVCK QLIALCCYHA KHCQENKCPV PFCLNIKHKL RQQQIQHRLQ
1861 QAQLMRRRMA TMNTRNVPQQ SLPSPTSAPP GTPTQQPSTP QTPQPPAQPQ PSPVSMSPAG
1921 FPSVARTQPP TTVSTGKPTS QVPAPPPPAQ PPPAAVEAAR QIEREAQQQQ HLYRVNINNS
1981 MPPGRTGMGT PGSQMAPVSL NVPRPNQVSG PVMPSMPPGQ WQQAPLPQQQ PMPGLPRPVI
2041 SMQAQAAVAG PRMPSVQPPR SISPSALQDL LRTLKSPSSP QQQQQVLNIL KSNPQLMAAF
2101 IKQRTAKYVA NQPGMQPQPG LQSQPGMQPQ PGMHQQPSLQ NLNAMQAGVP RPGVPPQQQA
2161 MGGLNPQGQA LNIMNPGHNP NMASMNPQYR EMLRRQLLQQ QQQQQQQQQQ QQQQQQGSAG
2221 MAGGMAGHGQ FQQPQGPGGY PPAMQQQQRM QQHLPLQGSS MGQMAAQMGQ LGQMGQPGLG
2281 ADSTPNIQQA LQQRILQQQQ MKQQIGSPGQ PNPMSPQQHM LSGQPQASHL PGQQIATSLS
2341 NQVRSPAPVQ SPRPQSQPPH SSPSPRIQPQ PSPHHVSPQT GSPHPGLAVT MASSIDQGHL
2401 GNPEQSAMLP QLNTPSRSAL SSELSLVGDT TGDTLEKFVE GL

In certain embodiments, a mutation of the disclosure may occur in a sequence encoding the CREB Binding Protein (CREBBP) HAT, including the nucleotide sequence encoding CREBBP (below, corresponding to GenBank Accession No. NM_001079846, defined as Homo sapiens CREB binding protein (CREBBP), transcript variant 2, mRNA; and identified as SEQ ID NO: 25).

1 CTGCGGGGCG CTGTTGCTGT GGCTGAGATT TGGCCGCCGC CTCCCCCACC CGGCCTGCGC
61 CCTCCCTCTC CCTCGGCGCC CGCCCGCCCG CTCGCGGCCC GCGCTCGCTC CTCTCCCTCG
121 CAGCCGGCAG GGCCCCCGAC CCCCGTCCGG GCCCTCGCCG GCCCGGCCGC CCGTGCCCGG
181 GGCTGTTTTC GCGAGCAGGT GAAAATGGCT GAGAACTTGC TGGACGGACC GCCCAACCCC
241 AAAAGAGCCA AACTCAGCTC GCCCGGTTTC TCGGCGAATG ACAGCACAGA TTTTGGATCA
301 TTGTTTGACT TGGAAAATGA TCTTCCTGAT GAGCTGATAC CCAATGGAGG AGAATTAGGC
361 CTTTTAAACA GTGGGAACCT TGTTCCAGAT GCTGCTTCCA AACATAAACA ACTGTCGGAG
421 CTTCTACGAG GAGGCAGCGG CTCTAGTATC AACCCAGGAA TAGGAAATGT GAGCGCCAGC
481 AGCCCCGTGC AGCAGGGCCT GGGTGGCCAG GCTCAAGGGC AGCCGAACAG TGCTAACATG
541 GCCAGCCTCA GTGCCATGGG CAAGAGCCCT CTGAGCCAGG GAGATTCTTC AGCCCCCAGC
601 CTGCCTAAAC AGGCAGCCAG CACCTCTGGG CCCACCCCCG CTGCCTCCCA AGCACTGAAT
661 CCGCAAGCAC AAAAGCAAGT GGGGCTGGCG ACTAGCAGCC CTGCCACGTC ACAGACTGGA
721 CCTGGTATCT GCATGAATGC TAACTTTAAC CAGACCCACC CAGGCCTCCT CAATAGTAAC
781 TCTGGCCATA GCTTAATTAA TCAGGCTTCA CAAGGGCAGG CGCAAGTCAT GAATGGATCT
841 CTTGGGGCTG CTGGCAGAGG AAGGGGAGCT GGAATGCCGT ACCCTACTCC AGCCATGCAG
901 GGCGCCTCGA GCAGCGTGCT GGCTGAGACC CTAACGCAGG TTTCCCCGCA AATGACTGGT
961 CACGCGGGAC TGAACACCGC ACAGGCAGGA GGCATGGCCA AGATGGGAAT AACTGGGAAC
1021 ACAAGTCCAT TTGGACAGCC CTTTAGTCAA GCTGGAGGGC AGCCAATGGG AGCCACTGGA
1081 GTGAACCCCC AGTTAGCCAG CAAACAGAGC ATGGTCAACA GTTTGCCCAC CTTCCCTACA
1141 GATATCAAGA ATACTTCAGT CACCAACGTG CCAAATATGT CTCAGATGCA AACATCAGTG
1201 GGAATTGTAC CCACACAAGC AATTGCAACA GGCCCCACTG CAGATCCTGA AAAACGCAAA
1261 CTGATACAGC AGCAGCTGGT TCTACTGCTT CATGCTCATA AGTGTCAGAG ACGAGAGCAA
1321 GCAAACGGAG AGGTTCGGGC CTGCTCGCTC CCGCATTGTC GAACCATGAA AAACGTTTTG
1381 AATCACATGA CGCATTGTCA GGCTGGGAAA GCCTGCCAAG CCATCCTGGG GTCTCCAGCT
1441 AGTGGAATTC AAAACACAAT TGGTTCTGTT GGCACAGGGC AACAGAATGC CACTTCTTTA
1501 AGTAACCCAA ATCCCATAGA CCCCAGCTCC ATGCAGCGAG CCTATGCTGC TCTCGGACTC
1561 CCCTACATGA ACCAGCCCCA GACGCAGCTG CAGCCTCAGG TTCCTGGCCA GCAACCAGCA
1621 CAGCCTCAAA CCCACCAGCA GATGAGGACT CTCAACCCCC TGGGAAATAA TCCAATGAAC
1681 ATTCCAGCAG GAGGAATAAC AACAGATCAG CAGCCCCCAA ACTTGATTTC AGAATCAGCT
1741 CTTCCGACTT CCCTGGGGGC CACAAACCCA CTGATGAACG ATGGCTCCAA CTCTGGTAAC
1801 ATTGGAACCC TCAGCACTAT ACCAACAGCA GCTCCTCCTT CTAGCACCGG TGTAAGGAAA
1861 GGCTGGCACG AACATGTCAC TCAGGACCTG CGGAGCCATC TAGTGCATAA ACTCGTCCAA
1921 GCCATCTTCC CAACACCTGA TCCCGCAGCT CTAAAGGATC GCCGCATGGA AAACCTGGTA
1981 GCCTATGCTA AGAAAGTGGA AGGGGACATG TACGAGTCTG CCAACAGCAG GGATGAATAT
2041 TATCACTTAT TAGCAGAGAA AATCTACAAG ATACAAAAAG AACTAGAAGA AAAACGGAGG
2101 TCGCGTTTAC ATAAACAAGG CATCTTGGGG AACCAGCCAG CCTTACCAGC CCCGGGGGCT
2161 CAGCCCCCTG TGATTCCACA GGCACAACCT GTGAGACCTC CAAATGGACC CCTGTCCCTG
2221 CCAGTGAATC GCATGCAAGT TTCTCAAGGG ATGAATTCAT TTAACCCCAT GTCCTTGGGG
2281 AACGTCCAGT TGCCACAAGC ACCCATGGGA CCTCGTGCAG CCTCCCCAAT GAACCACTCT
2341 GTCCAGATGA ACAGCATGGG CTCAGTGCCA GGGATGGCCA TTTCTCCTTC CCGAATGCCT
2401 CAGCCTCCGA ACATGATGGG TGCACACACC AACAACATGA TGGCCCAGGC GCCCGCTCAG
2461 AGCCAGTTTC TGCCACAGAA CCAGTTCCCG TCATCCAGCG GGGCGATGAG TGTGGGCATG
2521 GGGCAGCCGC CAGCCCAAAC AGGCGTGTCA CAGGGACAGG TGCCTGGTGC TGCTCTTCCT
2581 AACCCTCTCA ACATGCTGGG GCCTCAGGCC AGCCAGCTAC CTTGCCCTCC AGTGACACAG
2641 TCACCACTGC ACCCAACACC GCCTCCTGCT TCCACGGCTG CTGGCATGCC ATCTCTCCAG
2701 CACACGACAC CACCTGGGAT GACTCCTCCC CAGCCAGCAG CTCCCACTCA GCCATCAACT
2761 CCTGTGTCGT CTTCCGGGCA GACTCCCACC CCGACTCCTG GCTCAGTGCC CAGTGCTACC
2821 CAAACCCAGA GCACCCCTAC AGTCCAGGCA GCAGCCCAGG CCCAGGTGAC CCCGCAGCCT
2881 CAAACCCCAG TTCAGCCCCC GTCTGTGGCT ACCCCTCAGT CATCGCAGCA ACAGCCGACG
2941 CCTGTGCACG CCCAGCCTCC TGGCACACCG CTTTCCCAGG CAGCAGCCAG CATTGATAAC
3001 AGAGTCCCTA CCCCCTCCTC GGTGGCCAGC GCAGAAACCA ATTCCCAGCA GCCAGGACCT
3061 GACGTACCTG TGCTGGAAAT GAAGACGGAG ACCCAAGCAG AGGACACTGA GCCCGATCCT
3121 GGTGAATCCA AAGGGGAGCC CAGGTCTGAG ATGATGGAGG AGGATTTGCA AGGAGCTTCC
3181 CAAGTTAAAG AAGAAACAGA CATAGCAGAG CAGAAATCAG AACCAATGGA AGTGGATGAA
3241 AAGAAACCTG AAGTGAAAGT AGAAGTTAAA GAGGAAGAAG AGAGTAGCAG TAACGGCACA
3301 GCCTCTCAGT CAACATCTCC TTCGCAGCCG CGCAAAAAAA TCTTTAAACC AGAGGAGTTA
3361 CGCCAGGCCC TCATGCCAAC CCTAGAAGCA CTGTATCGAC AGGACCCAGA GTCATTACCT
3421 TTCCGGCAGC CTGTAGATCC CCAGCTCCTC GGAATTCCAG ACTATTTTGA CATCGTAAAG
3481 AATCCCATGG ACCTCTCCAC CATCAAGCGG AAGCTGGACA CAGGGCAATA CCAAGAGCCC
3541 TGGCAGTACG TGGACGACGT CTGGCTCATG TTCAACAATG CCTGGCTCTA TAATCGCAAG
3601 ACATCCCGAG TCTATAAGTT TTGCAGTAAG CTTGCAGAGG TCTTTGAGCA GGAAATTGAC
3661 CCTGTCATGC AGTCCCTTGG ATATTGCTGT GGACGCAAGT ATGAGTTTTC CCCACAGACT
3721 TTGTGCTGCT ATGGGAAGCA GCTGTGTACC ATTCCTCGCG ATGCTGCCTA CTACAGCTAT
3781 CAGAATAGGT ATCATTTCTG TGAGAAGTGT TTCACAGAGA TCCAGGGCGA GAATGTGACC
3841 CTGGGTGACG ACCCTTCACA GCCCCAGACG ACAATTTCAA AGGATCAGTT TGAAAAGAAG
3901 AAAAATGATA CCTTAGACCC CGAACCTTTC GTTGATTGCA AGGAGTGTGG CCGGAAGATG
3961 CATCAGATTT GCGTTCTGCA CTATGACATC ATTTGGCCTT CAGGTTTTGT GTGCGACAAC
4021 TGCTTGAAGA AAACTGGCAG ACCTCGAAAA GAAAACAAAT TCAGTGCTAA GAGGCTGCAG
4081 ACCACAAGAC TGGGAAACCA CTTGGAAGAC CGAGTGAACA AATTTTTGCG GCGCCAGAAT
4141 CACCCTGAAG CCGGGGAGGT TTTTGTCCGA GTGGTGGCCA GCTCAGACAA GACGGTGGAG
4201 GTCAAGCCCG GGATGAAGTC ACGGTTTGTG GATTCTGGGG AAATGTCTGA ATCTTTCCCA
4261 TATCGAACCA AAGCTCTGTT TGCTTTTGAG GAAATTGACG GCGTGGATGT CTGCTTTTTT
4321 GGAATGCACG TCCAAGAATA CGGCTCTGAT TGCCCCCCTC CAAACACGAG GCGTGTGTAC
4381 ATTTCTTATC TGGATAGTAT TCATTTCTTC CGGCCACGTT GCCTCCGCAC AGCCGTTTAC
4441 CATGAGATCC TTATTGGATA TTTAGAGTAT GTGAAGAAAT TAGGGTATGT GACAGGGCAC
4501 ATCTGGGCCT GTCCTCCAAG TGAAGGAGAT GATTACATCT TCCATTGCCA CCCACCTGAT
4561 CAAAAAATAC CCAAGCCAAA ACGACTGCAG GAGTGGTACA AAAAGATGCT GGACAAGGCG
4621 TTTGCAGAGC GGATCATCCA TGACTACAAG GATATTTTCA AACAAGCAAC TGAAGACAGG
4681 CTCACCAGTG CCAAGGAACT GCCCTATTTT GAAGGTGATT TCTGGCCCAA TGTGTTAGAA
4741 GAGAGCATTA AGGAACTAGA ACAAGAAGAA GAGGAGAGGA AAAAGGAAGA GAGCACTGCA
4801 GCCAGTGAAA CCACTGAGGG CAGTCAGGGC GACAGCAAGA ATGCCAAGAA GAAGAACAAC
4861 AAGAAAACCA ACAAGAACAA AAGCAGCATC AGCCGCGCCA ACAAGAAGAA GCCCAGCATG
4921 CCCAACGTGT CCAATGACCT GTCCCAGAAG CTGTATGCCA CCATGGAGAA GCACAAGGAG
4981 GTCTTCTTCG TGATCCACCT GCACGCTGGG CCTGTCATCA ACACCCTGCC CCCCATCGTC
5041 GACCCCGACC CCCTGCTCAG CTGTGACCTC ATGGATGGGC GCGACGCCTT CCTCACCCTC
5101 GCCAGAGACA AGCACTGGGA GTTCTCCTCC TTGCGCCGCT CCAAGTGGTC CACGCTCTGC
5161 ATGCTGGTGG AGCTGCACAC CCAGGGCCAG GACCGCTTTG TCTACACCTG CAACGAGTGC
5221 AAGCACCACG TGGAGACGCG CTGGCACTGC ACTGTGTGCG AGGACTACGA CCTCTGCATC
5281 AACTGCTATA ACACGAAGAG CCATGCCCAT AAGATGGTGA AGTGGGGGCT GGGCCTGGAT
5341 GACGAGGGCA GCAGCCAGGG CGAGCCACAG TCAAAGAGCC CCCAGGAGTC ACGCCGGCTG
5401 AGCATCCAGC GCTGCATCCA GTCGCTGGTG CACGCGTGCC AGTGCCGCAA CGCCAACTGC
5461 TCGCTGCCAT CCTGCCAGAA GATGAAGCGG GTGGTGCAGC ACACCAAGGG CTGCAAACGC
5521 AAGACCAACG GGGGCTGCCC GGTGTGCAAG CAGCTCATCG CCCTCTGCTG CTACCACGCC
5581 AAGCACTGCC AAGAAAACAA ATGCCCCGTG CCCTTCTGCC TCAACATCAA ACACAAGCTC
5641 CGCCAGCAGC AGATCCAGCA CCGCCTGCAG CAGGCCCAGC TCATGCGCCG GCGGATGGCC
5701 ACCATGAACA CCCGCAACGT GCCTCAGCAG AGTCTGCCTT CTCCTACCTC AGCACCGCCC
5761 GGGACCCCCA CACAGCAGCC CAGCACACCC CAGACGCCGC AGCCCCCTGC CCAGCCCCAA
5821 CCCTCACCCG TGAGCATGTC ACCAGCTGGC TTCCCCAGCG TGGCCCGGAC TCAGCCCCCC
5881 ACCACGGTGT CCACAGGGAA GCCTACCAGC CAGGTGCCGG CCCCCCCACC CCCGGCCCAG
5941 CCCCCTCCTG CAGCGGTGGA AGCGGCTCGG CAGATCGAGC GTGAGGCCCA GCAGCAGCAG
6001 CACCTGTACC GGGTGAACAT CAACAACAGC ATGCCCCCAG GACGCACGGG CATGGGGACC
6061 CCGGGGAGCC AGATGGCCCC CGTGAGCCTG AATGTGCCCC GACCCAACCA GGTGAGCGGG
6121 CCCGTCATGC CCAGCATGCC TCCCGGGCAG TGGCAGCAGG CGCCCCTTCC CCAGCAGCAG
6181 CCCATGCCAG GCTTGCCCAG GCCTGTGATA TCCATGCAGG CCCAGGCGGC CGTGGCTGGG
6241 CCCCGGATGC CCAGCGTGCA GCCACCCAGG AGCATCTCAC CCAGCGCTCT GCAAGACCTG
6301 CTGCGGACCC TGAAGTCGCC CAGCTCCCCT CAGCAGCAAC AGCAGGTGCT GAACATTCTC
6361 AAATCAAACC CGCAGCTAAT GGCAGCTTTC ATCAAACAGC GCACAGCCAA GTACGTGGCC
6421 AATCAGCCCG GCATGCAGCC CCAGCCTGGC CTCCAGTCCC AGCCCGGCAT GCAACCCCAG
6481 CCTGGCATGC ACCAGCAGCC CAGCCTGCAG AACCTGAATG CCATGCAGGC TGGCGTGCCG
6541 CGGCCCGGTG TGCCTCCACA GCAGCAGGCG ATGGGAGGCC TGAACCCCCA GGGCCAGGCC
6601 TTGAACATCA TGAACCCAGG ACACAACCCC AACATGGCGA GTATGAATCC ACAGTACCGA
6661 GAAATGTTAC GGAGGCAGCT GCTGCAGCAG CAGCAGCAAC AGCAGCAGCA ACAACAGCAG
6721 CAACAGCAGC AGCAGCAAGG GAGTGCCGGC ATGGCTGGGG GCATGGCGGG GCACGGCCAG
6781 TTCCAGCAGC CTCAAGGACC CGGAGGCTAC CCACCGGCCA TGCAGCAGCA GCAGCGCATG
6841 CAGCAGCATC TCCCCCTCCA GGGCAGCTCC ATGGGCCAGA TGGCGGCTCA GATGGGACAG
6901 CTTGGCCAGA TGGGGCAGCC GGGGCTGGGG GCAGACAGCA CCCCCAACAT CCAGCAAGCC
6961 CTGCAGCAGC GGATTCTGCA GCAACAGCAG ATGAAGCAGC AGATTGGGTC CCCAGGCCAG
7021 CCGAACCCCA TGAGCCCCCA GCAACACATG CTCTCAGGAC AGCCACAGGC CTCGCATCTC
7081 CCTGGCCAGC AGATCGCCAC GTCCCTTAGT AACCAGGTGC GGTCTCCAGC CCCTGTCCAG
7141 TCTCCACGGC CCCAGTCCCA GCCTCCACAT TCCAGCCCGT CACCACGGAT ACAGCCCCAG
7201 CCTTCGCCAC ACCACGTCTC ACCCCAGACT GGTTCCCCCC ACCCCGGACT CGCAGTCACC
7261 ATGGCCAGCT CCATAGATCA GGGACACTTG GGGAACCCCG AACAGAGTGC AATGCTCCCC
7321 CAGCTGAACA CCCCCAGCAG GAGTGCGCTG TCCAGCGAAC TGTCCCTGGT CGGGGACACC
7381 ACGGGGGACA CGCTAGAGAA GTTTGTGGAG GGCTTGTAGC ATTGTGAGAG CATCACCTTT
7441 TCCCTTTCAT GTTCTTGGAC CTTTTGTACT GAAAATCCAG GCATCTAGGT TCTTTTTATT
7501 CCTAGATGGA ACTGCGACTT CCGAGCCATG GAAGGGTGGA TTGATGTTTA AAGAAACAAT
7561 ACAAAGAATA TATTTTTTTG TTAAAAACCA GTTGATTTAA ATATCTGGTC TCTCTCTTTG
5621 GTTTTTTTTT GGCGGGGGGG TGGGGGGGGT TCTTTTTTTT CCGTTTTGTT TTTGTTTGGG
7681 GGGAGGGGGG TTTTGTTTGG ATTCTTTTTG TCGTCATTGC TGGTGACTCA TGCCTTTTTT
7741 TAACGGGAAA AACAAGTTCA TTATATTCAT ATTTTTTATT TGTATTTTCA AGACTTTAAA
7801 CATTTATGTT TAAAAGTAAG AAGAAAAATA ATATTCAGAA CTGATTCCTG AAATAATGCA
7861 AGCTTATAAT GTATCCCGAT AACTTTGTGA TGTTTCGGGA AGATTTTTTT CTATAGTGAA
7921 CTCTGTGGGC GTCTCCCAGT ATTACCCTGG ATGATAGGAA TTGACTCCGG CGTGCACACA
7981 CGTACACACC CACACACATC TATCTATACA TAATGGCTGA AGCCAAACTT GTCTTGCAGA
8041 TGTAGAAATT GTTGCTTTGT TTCTCTGATA AAACTGGTTT TAGACAAAAA ATAGGGATGA
8101 TCACTCTTAG ACCATGCTAA TGTTACTAGA GAAGAAGCCT TCTTTTCTTT CTTCTATGTG
8161 AAACTTGAAA TGAGGAAAAG CAATTCTAGT GTAAATCATG CAAGCGCTCT AATTCCTATA
8221 AATACGAAAC TCGAGAAGAT TCAATCACTG TATAGAATGG TAAAATACCA ACTCATTTCT
8281 TATATCATAT TGTTAAATAA ACTGTGTGCA ACAGACAAAA AGGGTGGTCC TTCTTGAATT
8341 CATGTACATG GTATTAACAC TTAGTGTTCG GGGTTTTTTG TTATGAAAAT GCTGTTTTCA
8401 ACATTGTATT TGGACTATGC ATGTGTTTTT TCCCCATTGT ATATAAAGTA CCGCTTAAAA
8461 TTGATATAAA TTACTGAGGT TTTTAACATG TATTCTGTTC TTTAAGATCC CTGTAAGAAT
8521 GTTTAAGGTT TTTATTTATT TATATATATT TTTTGAGTCT GTTCTTTGTA AGACATGGTT
8581 CTGGTTGTTC GCTCATAGCG GAGAGGCTGG GGCTGCGGTT GTGGTTGTGG CGGCGTGGGT
8641 GGTGGCTGGG AACTGTGGCC CAGGCTTAGC GGCCGCCCGG AGGCTTTTCT TCCCGGAGAC
8701 TGAGGTGGGC GACTGAGGTG GGCGGCTCAG CGTTGGCCCC ACACATTCGA GGCTCACAGG
8761 TGATTGTCGC TCACACAGTT AGGGTCGTCA GTTGGTCTGA AACTGCATTT GGCCCACTCC
8821 TCCATCCTCC CTGTCCGTCG TAGCTGCCAC CCCCAGAGGC GGCGCTTCTT CCCGTGTTCA
8881 GGCGGCTCCC CCCCCCCGTA CACGACTCCC AGAATCTGAG GCAGAGAGTG CTCCAGGCTC
8941 GCGAGGTGCT TTCTGACTTC CCCCCAAATC CTGCCGCTGC CGCGCAGCAT GTCCCGTGTG
9001 GCGTTTGAGG AAATGCTGAG GGACAGACAC CTTGGAGCAC CAGCTCCGGT CCCTGTTACA
9061 GTGAGAAAGG TCCCCCACTT CGGGGGATAC TTGCACTTAG CCACATGGTC CTGCCTCCCT
9121 TGGAGTCCAG TTCCAGGCTC CCTTACTGAG TGGGTGAGAC AAGTTCACAA AAACCGTAAA
9181 ACTGAGAGGA GGACCATGGG CAGGGGAGCT GAAGTTCATC CCCTAAGTCT ACCACCCCCA
9241 GCACCCAGAG AACCCACTTT ATCCCTAGTC CCCCAACAAA GGCTGGTCTA GGTGGGGGTG
9301 ATGGTAATTT TAGAAATCAC GCCCCAAATA GCTTCCGTTT GGGCCCTTAC ATTCACAGAT
9361 AGGTTTTAAA TAGCTGAATA CTTGGTTTGG GAATCTGAAT TCGAGGAACC TTTCTAAGAA
9421 GTTGGAAAGG TCCGATCTAG TTTTAGCACA GAGCTTTGAA CCTTGAGTTA TAAAATGCAG
9481 AATAATTCAA GTAAAAATAA GACCACCATC TGGCACCCCT GACCAGCCCC CATTCACCCC
9541 ATCCCAGGAG GGGAAGCACA GGCCGGGCCT CCGGTGGAGA TTGCTGCCAC TGCTCGGCCT
9601 GCTGGGTTCT TAACCTCCAG TGTCCTCTTC ATCTTTTCCA CCCGTAGGGA AACCTTGAGC
9661 CATGTGTTCA AACAAGAAGT GGGGCTAGAG CCCGAGAGCA GCAGCTCTAA GCCCACACTC
9721 AGAAAGTGGC GCCCTCCTGG TTGTGCAGCC TTTTAATGTG GGCAGTGGAG GGGCCTCTGT
9781 TTCAGGTTAT CCTGGAATTC AAAACGTTAT GTACCAACCT CATCCTCTTT GGAGTCTGCA
9841 TCCTGTGCAA CCGTCTTGGG CAATCCAGAT GTCGAAGGAT GTGACCGAGA GCATGGTCTG
9901 TGGATGCTAA CCCTAAGTTT GTCGTAAGGA AATTTCTGTA AGAAACCTGG AAAGCCCCAA
9961 CGCTGTGTCT CATGCTGTAT ACTTAAGAGG AGAAGAAAAA GTCCTATATT TGTGATCAAA
10021 AAGAGGAAAC TTGAAATGTG ATGGTGTTTA TAATAAAAGA TGGTAAAACT ACTTGGATTC
10081 AAA

In certain embodiments, a mutation of the disclosure may occur in a sequence encoding the CREB Binding Protein (CREBBP) HAT, including the amino acid sequence encoding CREBBP (below, corresponding to GenBank Accession No. NP_001073315.1, defined as Homo sapiens CREB-binding protein isoform b; and identified as SEQ ID NO: 26).

MAENLLDGPPNPKRAKLSSPGFSANDSTDFGSLFDLENDLPDELIPNGGE
LGLLNSGNLVPDAASKHKQLSELLRGGSGSSINPGIGNVSASSPVQQGLG
GQAQGQPNSANMASLSAMGKSPLSQGDSSAPSLPKQAASTSGPTPAASQA
LNPQAQKQVGLATSSPATSQTGPGICMNANFNQTHPGLLNSNSGHSLINQ
ASQGQAQVMNGSLGAAGRGRGAGMPYPTPAMQGASSSVLAETLTQVSPQM
TGHAGLNTAQAGGMAKMGITGNTSPFGQPFSQAGGQPMGATGVNPQLASK
QSMVNSLPTFPTDIKNTSVTNVPNMSQMQTSVGIVPTQAIATGPTADPEK
RKLIQQQLVLLLHAHKCQRREQANGEVRACSLPHCRTMKNVLNHMTHCQA
GKACQAILGSPASGIQNTIGSVGTGQQNATSLSNPNPIDPSSMQRAYAAL
GLPYMNQPQTQLQPQVPGQQPAQPQTHQQMRTLNPLGNNPMNIPAGGITT
DQQPPNLISESALPTSLGATNPLMNDGSNSGNIGTLSTIPTAAPPSSTGV
RKGWHEHVTQDLRSHLVHKLVQAIFPTPDPAALKDRRMENLVAYAKKVEG
DMYESANSRDEYYHLLAEKIYKIQKELEEKRRSRLHKQGILGNQPALPAP
GAQPPVIPQAQPVRPPNGPLSLPVNRMQVSQGMNSFNPMSLGNVQLPQAP
MGPRAASPMNHSVQMNSMGSVPGMAISPSRMPQPPNMMGAHTNNMMAQAP
AQSQFLPQNQFPSSSGAMSVGMGQPPAQTGVSQGQVPGAALPNPLNMLGP
QASQLPCPPVTQSPLHPTPPPASTAAGMPSLQHTTPPGMTPPQPAAPTQP
STPVSSSGQTPTPTPGSVPSATQTQSTPTVQAAAQAQVTPQPQTPVQPPS
VATPQSSQQQPTPVHAQPPGTPLSQAAASIDNRVPTPSSVASAETNSQQP
GPDVPVLEMKTETQAEDTEPDPGESKGEPRSEMMEEDLQGASQVKEETDI
AEQKSEPMEVDEKKPEVKVEVKEEEESSSNGTASQSTSPSQPRKKIFKPE
ELRQALMPTLEALYRQDPESLPFRQPVDPQLLGIPDYFDIVKNPMDLSTI
KRKLDTGQYQEPWQYVDDVWLMFNNAWLYNRKTSRVYKFCSKLAEVFEQE
IDPVMQSLGYCCGRKYEFSPQTLCCYGKQLCTIPRDAAYYSYQNRYHFCE
KCFTEIQGENVTLGDDPSQPQTTISKDQFEKKKNDTLDPEPFVDCKECGR
KMHQICVLHYDIIWPSGFVCDNCLKKTGRPRKENKFSAKRLQTTRLGNHL
EDRVNKFLRRQNHPEAGEVFVRWASSDKTVEVKPGMKSRFVDSGEMSESF
PYRTKALFAFEEIDGVDVCFFGMHVQEYGSDCPPPNTRRVYISYLDSIHF
FRPRCLRTAVYHEILIGYLEYVKKLGYVTGHIWACPPSEGDDYIFHCHPP
DQKIPKPKRLQEWYKKMLDKAFAERIIHDYKDIFKQATEDRLTSAKELPY
FEGDFWPNVLEESIKELEQEEEERKKEESTAASETTEGSQGDSKNAKKKN
NKKTNKNKSSISRANKKKPSMPNVSNDLSQKLYATMEKHKEVFFVIHLHA
GPVINTLPPIVDPDPLLSCDLMDGRDAFLTLARDKHWEFSSLRRSKWSTL
CMLVELHTQGQDRFVYTCNECKHHVETRWHCTVCEDYDLCINCYNTKSHA
HKMVKWGLGLDDEGSSQGEPQSKSPQESRRLSIQRCIQSLVHACQCRNAN
CSLPSCQKMKRVVQHTKGCKRKTNGGCPVCKQLIALCCYHAKHCQENKCP
VPFCLNIKHKLRQQQIQHRLQQAQLMRRRMATMNTRNVPQQSLPSPTSAP
PGTPTQQPSTPQTPQPPAQPQPSPVSMSPAGFPSVARTQPPTTVSTGKPT
SQVPAPPPPAQPPPAAVEAARQIEREAQQQQHLYRVNINNSMPPGRTGMG
TPGSQMAPVSLNVPRPNQVSGPVMPSMPPGQWQQAPLPQQQPMPGLPRPV
ISMQAQAAVAGPRMPSVQPPRSISPSALQDLLRTLKSPSSPQQQQQVLNI
LKSNPQLMAAFIKQRTAKYVANQPGMQPQPGLQSQPGMQPQPGMHQQPSL
QNLNAMQAGVPRPGVPPQQQAMGGLNPQGQALNIMNPGHNPNMASMNPQY
REMLRRQLLQQQQQQQQQQQQQQQQQQGSAGMAGGMAGHGQFQQPQGPGG
YPPAMQQQQRMQQHLPLQGSSMGQMAAQMGQLGQMGQPGLGADSTPNIQQ
ALQQRILQQQQMKQQIGSPGQPNPMSPQQHMLSGQPQASHLPGQQIATSL
SNQVRSPAPVQSPRPQSQPPHSSPSPRIQPQPSPHHVSPQTGSPHPGLAV
TMASSIDQGHLGNPEQSAMLPQLNTPSRSALSSELSLVGDTTGDTLEKFV
EGL

### Next Generation Sequencing

The compounds of the disclosure are inhibitors of the histone methyltransferase EZH2 for use in the treatment of patients with non-Hodgkin lymphoma (NHL), and in patients with certain genetically defined solid tumors. Activating EZH2 mutations present in NHL patients has been implicated to predict response to EZH2 inhibition (Knutson et al., Nat. Chem. Biol. 2012; 8: 890-896, the content of which is incorporated herein by reference in its entirety). Furthermore, a phase 1 clinical trial of tazemetostat demonstrated clinical responses in both EZH2 mutant and wild type patients (ClinicalTrials.gov identifier: NCT01897571). However, the impact of somatic mutations other than EZH2 on likelihood of response to tazemetostat in NHL patients is currently unknown. In some aspects, the present disclosure provides a multi-gene NHL targeted next generation sequencing (NGS) panel (e.g., a 39-gene panel or a 62-gene panel, or a panel combining a plurality of genes or gene products referred to herein) capable of analyzing samples from malignant cells, tissues, or body fluids, e.g., archive tissue or cell-free circulating tumor DNA (ctDNA) isolated from plasma. In some aspects, the NGS panel is capable of identifying molecular variants, including specific somatic sequence mutations (single base and insertion/deletion, e.g., EZH2), amplifications (e.g., BLC2) and translocations (e.g., BCL2 and MYC) in the tumor and ctDNA samples down to variant allele frequencies of 2% and 0.1% for archive and ctDNA respectively. For example, molecular variants associated with positive (e.g., EZH2, STAT6, MYD88, and SOCS1 mutations) and negative (e.g., MYC and HIST1H1E mutations) clinical responses to tazemetostat treatment were identified. Furthermore, sequencing of phase 1 NHL patients utilizing a 62 gene NHL NGS panel revealed a complex genetic landscape with epigenetic modifiers CREBBP and KMT2D representing the most frequently mutated genes in this sample set. Further aspects of the disclosure provide for an NGS panel with the ability to determine molecular profiles using ctDNA that enables patient characterization where archive tumor tissue or DNA is absent or limiting. Additionally, profiling ctDNA enables longitudinal monitoring of a patient’s mutation burden without the need for tumor biopsies.

Without wishing to be bound by theory, mutations identified by the NGS panel disclosed herein, may be used for patient stratification. Accordingly, in some embodiments, the disclosure provides a method of selecting a patient for cancer treatment if the patient has one or more mutations disclosed herein. In some embodiments, the patient selected for the cancer treatment has two or more (e.g., two, three, four, five, six, seven, eight, or more) mutations disclosed herein.

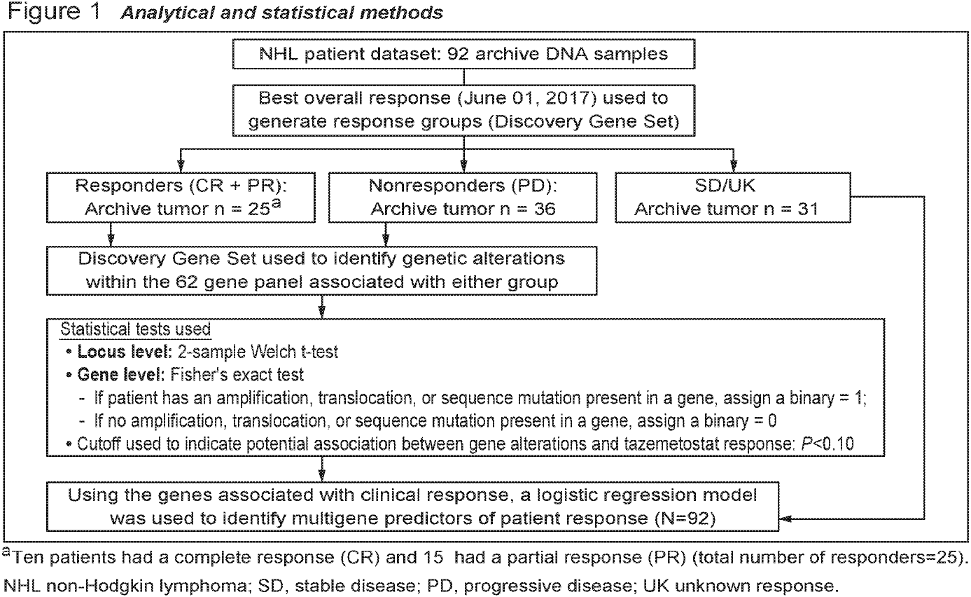

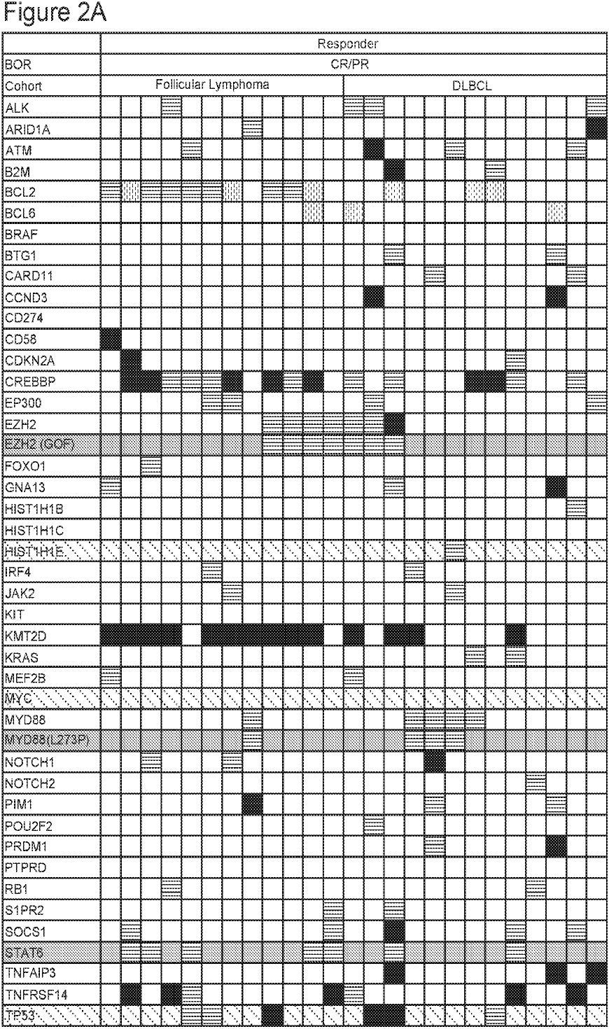

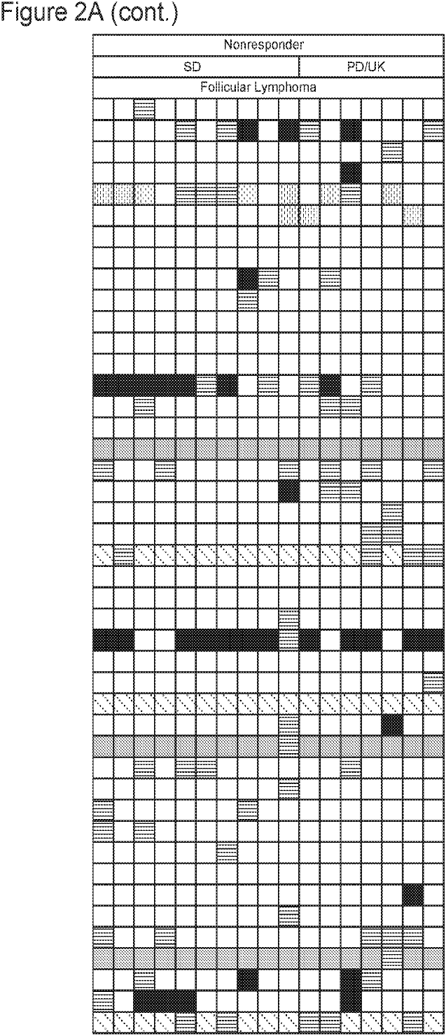

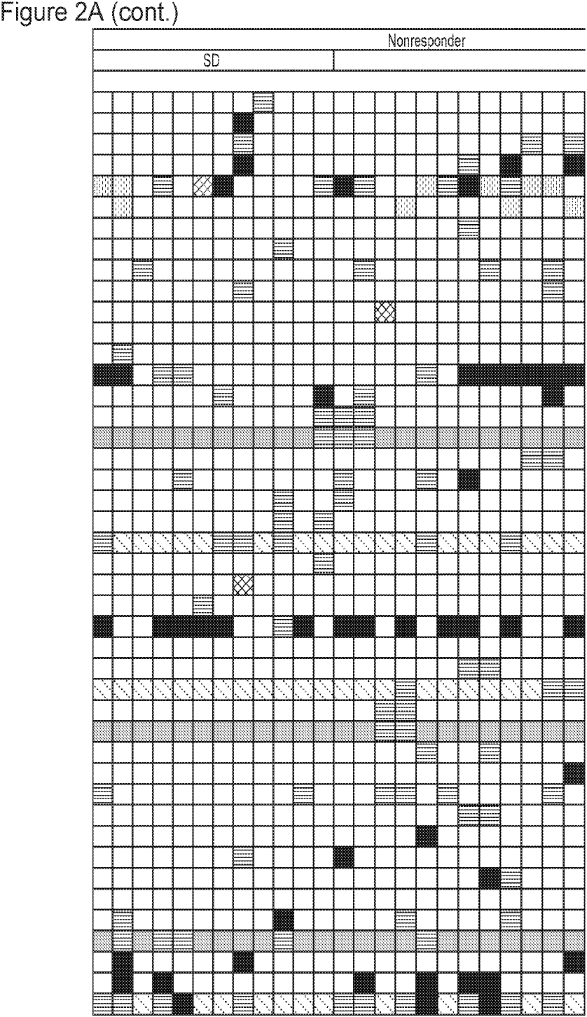

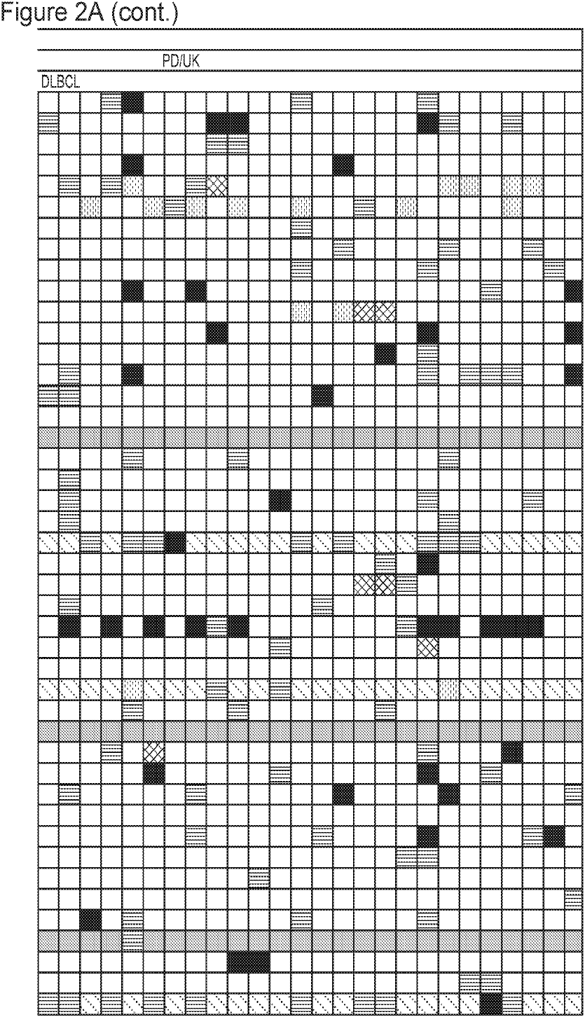

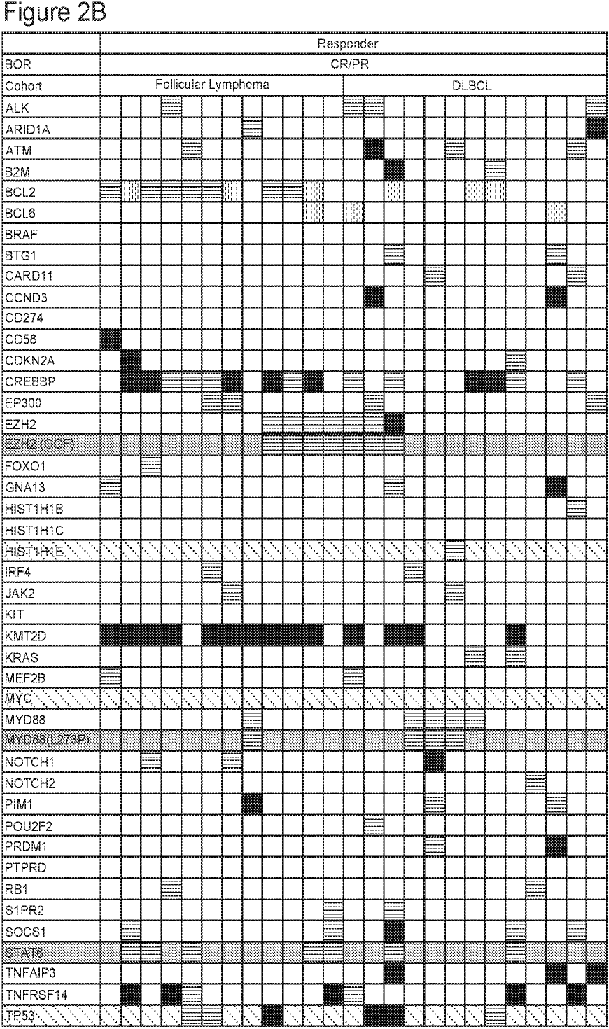

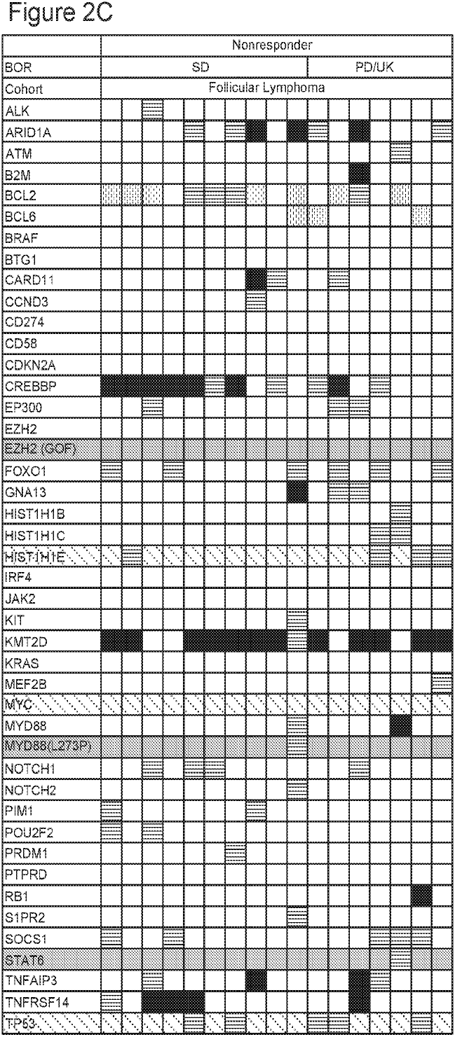

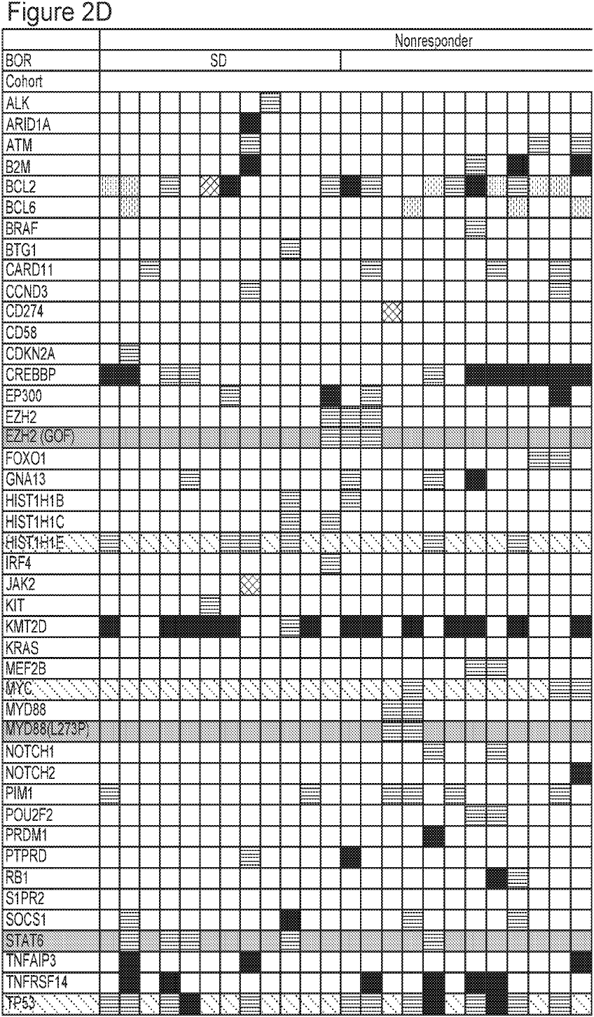

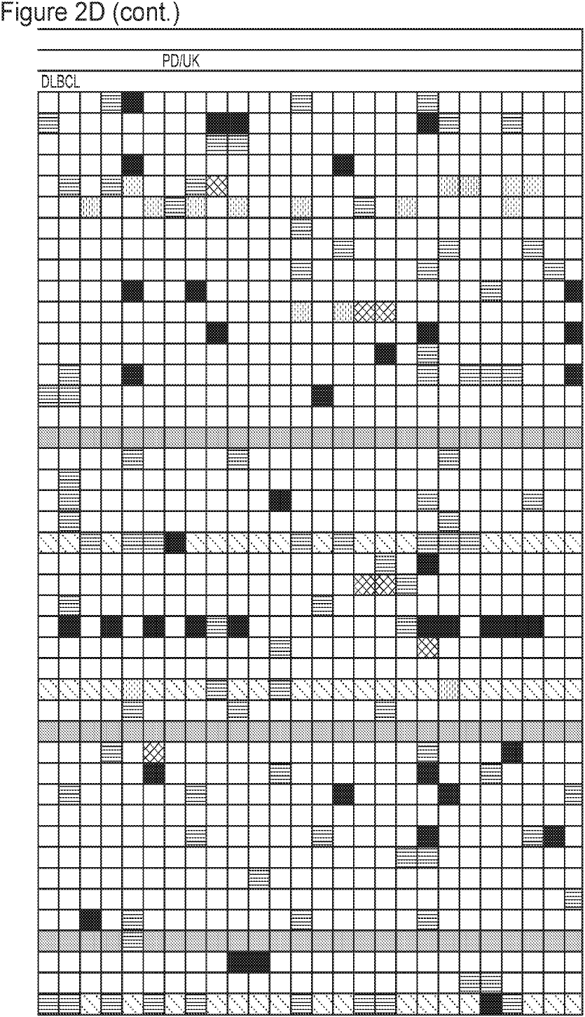

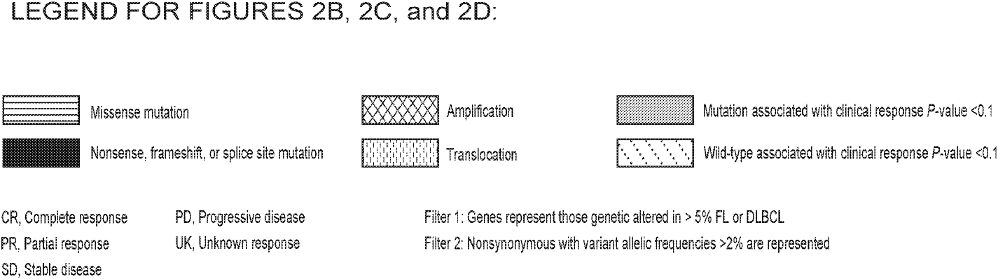

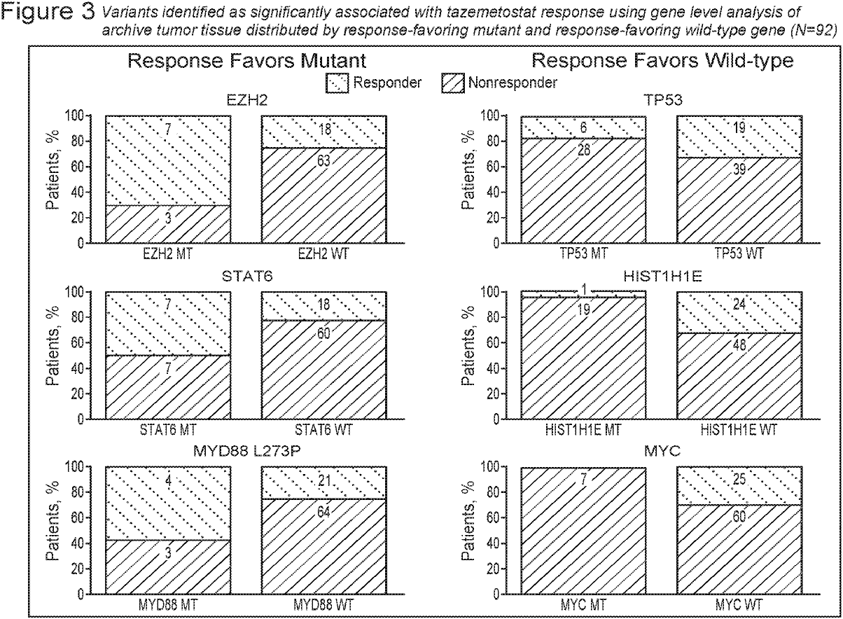

In some embodiments, a method is provided in which a subject having cancer is selected for treatment with an EZH2 inhibitor, e.g., an EZH2 inhibitor disclosed herein, based on the presence of one or more mutations associated with a positive response to such treatment in the subject, e.g., as determined by ctDNA analysis. In some embodiments, a mutation (or a combination of two or more mutations) associated with a positive response is a mutation (or a combination of mutations) that is present only in patients who responded with complete or partial response or, in some embodiments, with stable disease in any of the studies presented herein, e.g., those summarized in FIGS. 1-3. In some embodiments, a mutation (or a combination of two or more mutations) associated with a positive response is a mutation (or a combination of mutations) that is not randomly distributed within the patient population examined, but is overrepresented in those patients who responded with a complete or partial response or, in some embodiments, stable disease, in any of the studies presented herein, e.g., those summarized in FIGS. 1-3. In some embodiments, a mutation (or combination of mutations) associated with a positive response is a mutation (or combination of mutations) that is overrepresented in the responding (CR, PR, or, in some embodiments, SD) patient population at least 2-fold, at least 3-fold, at least 4-fold, at least 5-fold, or at least 10-fold, as compared to the patient population that did not respond or responded with progressive disease (PD).

In some embodiments, a method is provided in which a subject having cancer is selected for treatment with an EZH2 inhibitor, e.g., an EZH2 inhibitor disclosed herein, based on the absence of one or more mutations associated with a negative response to such treatment in the subject, e.g., as determined by ctDNA analysis. In some embodiments, a mutation (or a combination of two or more mutations) associated with a negative response is a mutation (or a combination of mutations) that is present only in patients who did not respond or responded with progressive disease (PD) in any of the studies presented herein, e.g., those summarized in FIGS. 1-3. In some embodiments, a mutation (or a combination of two or more mutations) associated with a negative response is a mutation (or a combination of mutations) that is not randomly distributed within the patient population examined, but is overrepresented in those patients who did not respond or responded with progressive disease in any of the studies presented herein, e.g., those summarized in FIGS. 1-3. In some embodiments, a mutation (or combination of mutations) associated with a negative response is a mutation (or combination of mutations) that is overrepresented in the non-responding or progressive disease (PD) patient population at least 2-fold, at least 3-fold, at least 4-fold, at least 5-fold, or at least 10-fold, as compared to the patient population that responded with CR, PR, or, in some embodiments, SD.

In some embodiments, a subject having cancer is selected for treatment with an EZH2 inhibitor, e.g., an EZH2 inhibitor disclosed herein, based on the presence of two or more (e.g., two, three, four, five, six, seven, eight, or more) mutations in the subject that match the mutations observed in a profile of a patient who exhibited a complete or partial response in any of the studies described herein (e.g., those summarized in FIGS. 1-3). In some embodiments, a subject having cancer is selected for treatment with an EZH2 inhibitor, e.g., an EZH2 inhibitor disclosed herein, based on the presence of a mutation profile (e.g., of two or more (e.g., two, three, four, five, six, seven, eight, or more)) mutations in the subject that match the mutation profile of a patient who exhibited a complete or partial response in any of the studies described herein (e.g., those summarized in FIGS. 1-3). Typically, a mutation in a gene or gene product (e.g., in a transcript, mRNA, or protein) is detected by comparing a given sequence with a reference sequence, e.g., a human reference genome sequence (e.g., human reference genome hg19), and identifying a mismatch in the sequence at hand as compared to the reference sequence.

In some embodiments, a subject having cancer is selected for treatment with an EZH2 inhibitor, e.g., an EZH2 inhibitor disclosed herein, based on the presence of two or more (e.g., two, three, four, five, six, seven, eight, or more) mutations in the subject that match the mutations observed in a profile of a patient who exhibited stable disease in any of the studies described herein (e.g., those summarized in FIGS. 1-3). In some embodiments, a subject having cancer is selected for treatment with an EZH2 inhibitor, e.g., an EZH2 inhibitor disclosed herein, based on the presence of a mutation profile (e.g., two or more (e.g., two, three, four, five, six, seven, eight, or more)) mutations in the subject that match the mutation profile of a patient who exhibited stable disease in any of the studies described herein (e.g., those summarized in FIGS. 1-3).

In some embodiments, methods of treating cancer is provided that comprises administering a therapeutically effective amount of an inhibitor of EZH2 to a subject in need thereof, wherein the subject has at least one mutation in one or more sequences encoding a gene or a gene product (e.g., a transcript, mRNA, or protein) listed in Tables 1-5, and/or FIGS. 1-3. In some embodiments, the subject has at least one mutation in in one or more sequences encoding: MYD88, STAT6A, SOCS1, MYC, HIST1H1E, ABL1, ACVR1, AKT1, AKT2, ALK, APC, AR, ARID1A, ARID1B, ASXL1, ATM, ATRX, AURKA, AXIN2, BAP1, BCL2, BCR, BLM, BMPR1A, BRAF, BRCA1, BRCA2, BRIP1, BTK, BUB1B, CALR, CBL, CCND1, CCNE1, CDC73, CDH1, CDK4, CDK6, CDKN1B, CDKN2A, CDKN2B, CDKN2C, CEBPA, CHEK2, CIC, CREBBP, CSF1R, CTNNB1, CYLD, DAXX, DDB2, DDR2, DICER1, DNMT3A, EGFR, EP300, ERBB2, ERBB3, ERBB4, ERCC1, ERCC2, ERCC3, ERCC4, ERCC5, ESR1, ETV1, ETV5, EWSR1, EXT1, EXT2, FANCA, FANCB, FANCC, FANCD2, FANCE, FANCF, FANCG, FANCI, FANCL, FANCM, FBXW7, FGFR1, FGFR2, FGFR3, FGFR4, FH, FLCN, FLT3, FLT4, FOXL2, GATA1, GATA2, GNA11, GNAQ, GNAS, GPC3, H3F3A, H3F3B, HNF1A, HRAS, IDH1, IDH2, IGF1R, IGF2R, IKZF1, JAK1, JAK2, JAK3, KDR, KIT, KRAS, MAML1, MAP2K1, MAP2K4, MDM2, MDM4, MED12, MEN1, MET, MLH1, MLL, MPL, MSH2, MSH6, MTOR, MUTYH, MYCL1, MYCN, NBN, NCOA3, NF1, NF2, NKX2-1, NOTCH1, NOTCH2, NOTCH3, NOTCH4, NPM1, NRAS, NTRK1, PALB2, PAX5, PBRM1, PDGFRA, PHOX2B, PIK3CA, PIK3R1, PMS1, PMS2, POLD1, POLE, POLH, POT1, PRKAR1A, PRSS1, PTCH1, PTEN, PTPN11, RAD51C, RAF1, RB1, RECQL4, RET, RNF43, ROS1, RUNX1, SBDS, SDHAF2, SDHB, SDHC, SDHD, SF3B1, SMAD2, SMAD3, SMAD4, SMARCB1, SMO, SRC, STAG2, STK11, SUFU, TERT, TET2, TGFBR2, TNFAIP3, TOP1, TP53, TSC1, TSC2, TSHR, VHL, WAS, WRN, WT1, XPA, XPC, and/or XRCC1. In some embodiments, the subject has at least one mutation in one or more sequences encoding ABL1, ACVR1, AKT1, AKT2, ALK, APC, AR, ARID1A, ARID1B, ASXL1, ATM, ATRX, AURKA, AXIN2, BAP1, BCL2, BCR, BLM, BMPR1A, BRAF, BRCA1, BRCA2, BRIP1, BTK, BUB1B, CALR, CBL, CCND1, CCNE1, CDC73, CDH1, CDK4, CDK6, CDKN1B, CDKN2A, CDKN2B, CDKN2C, CEBPA, CHEK2, CIC, CREBBP, CSF1R, CTNNB1, CYLD, DAXX, DDB2, DDR2, DICER1, DNMT3A, EGFR, EP300, ERBB2, ERBB3, ERBB4, ERCC1, ERCC2, ERCC3, ERCC4, ERCC5, ESR1, ETV1, ETV5, EWSR1, EXT1, EXT2, EZH2, FANCA, FANCB, FANCC, FANCD2, FANCE, FANCF, FANCG, FANCI, FANCL, FANCM, FBXW7, FGFR1, FGFR2, FGFR3, FGFR4, FH, FLCN, FLT3, FLT4, FOXL2, GATA1, GATA2, GNA11, GNAQ, GNAS, GPC3, H3F3A, H3F3B, HNF1A, HRAS, IDH1, IDH2, IGF1R, IGF2R, IKZF1, JAK1, JAK2, JAK3, KDR, KIT, KRAS, MAML1, MAP2K1, MAP2K4, MDM2, MDM4, MED12, MEN1, MET, MLH1, MLL, MPL, MSH2, MSH6, MTOR, MUTYH, MYCL1, MYCN, NBN, NCOA3, NF1, NF2, NKX2-1, NOTCH1, NOTCH2, NOTCH3, NOTCH4, NPM1, NRAS, NTRK1, PALB2, PAX5, PBRM1, PDGFRA, PHOX2B, PIK3CA, PIK3R1, PMS1, PMS2, POLD1, POLE, POLH, POT1, PRKAR1A, PRSS1, PTCH1, PTEN, PTPN11, RAD51C, RAF1, RB1, RECQL4, RET, RNF43, ROS1, RUNX1, SBDS, SDHAF2, SDHB, SDHC, SDHD, SF3B1, SMAD2, SMAD3, SMAD4, SMARCB1, SMO, SRC, STAG2, STK11, SUFU, TERT, TET2, TGFBR2, TNFAIP3, TOP1, TP53, TSC1, TSC2, TSHR, VHL, WAS, WRN, WT1, XPA, XPC, and/or XRCC1. In some embodiments, the subject has at least one mutation in one or more sequences encoding ARID1A, ATM, B2M, BCL2, BCL6, BCL7A, BRAF, BTG1, CARD11, CCND3, CD58, CD79B, CDKN2A, CREBBP, EP300, EZH2, FOXO1, GNA13, HIST1H1B, HIST1H1C, HIST1H1E, IKZF3, IRF4, ITPKB, KDM6A, KIT, KMT2D, KRAS, MEF2B, MYC, MYD88, NOTCH1, NOTCH2, NRAS, PIK3CA, PIM1, POU2F2, PRDM1, PTEN, PTPN1, PTPN11, PTPN6, PTPRD, RB1, S1PR2, SGK1, SMARCB1, SOCS1, STAT6, TBL1XR1, TNFAIP3, TNFRSF14, TP53, XPO1. In some embodiments, the subject has at least one mutation in one or more sequences encoding AKT1, ALK, ARID1A, ATM, B2M, BCL2, BCL6, BCL7A, BTG2, CARD11, CCND3, CD79B, CDKN2A, CREBBP, EP300, EZH2, FBXW7, FOXO1, HLA-C, HRAS, IKZF3, IRF4, KDM6A, KRAS, MEF2B, MYD88, NOTCH1, NPM1, NRAS, PIK3CA, PIM1, PRDM1, PTEN, RB1, RBBP4, SMARCB1, SUZ12, TNFRSF14, and/or TP53. In some embodiments, the subject has at least one mutation in one or more sequences encoding ALK, EWSR1, ROS1, BCL2, MLL, TMPRSS2, BCR, MYC, FGFR3, BRAF, NTRK1, TACC3, DNAJB1, PDGFRA, EGFR, PDGFRB, ETV1, PRKACA, ETV4, RAF1, ETV5, RARA, ETV6, RET. In some embodiments, the subject has at least one mutation in one or more sequences encoding ALK (Intron 19), BCL2 (MBR breakpoint region), BCL2 (MCR breakpoint region), BCL6, CD274, CIITA, MYC (entire Gene + 40kbp upstream), and/or PDCD1LG2. In some embodiments, the subject has at least one mutation in one or more sequences encoding BCL2, CD274 (PDL1), FOXP1, JAK2, KDM4C, PDCD1LG2 (PDL2), and/or REL. In some embodiments, the subject has at least one mutation in one or more sequences encoding ARID1A, ATM, B2M, BCL2, BCL6, BCL7A, BRAF, CARD11, CCND3, CD274 (PDL1), CD58, CD79B, CDKN2A, CIITA, CREBBP, EZH2 (non-Y646), EZH2 (Y646), EP300, FOXO1, FOXP1, GNA13, HIST1H1B, HIST1H1C, HIST1H1E, IRF4, IZKF3, JAK2, KDM4C, KDM6A, KIT, KMT2D, KRAS, MEF2B, MYC, MYD88, NOTCH1, NOTCH2, NRAS, PDCD1LG2 (PDL2), PIK3CA, PIM1, POU2F2, PRDM1, PTEN, PTPN11, PTPN6, PTPRD, REL, SOCS1, STAT6, TNFAIP3, TNFRSF14, and/or TP53. In some embodiments, the subject has at least one mutation in one or more sequences encoding ARID1A, B2M, BCL2, BCL6, CARD11, CCND3, CD274 (PDL1), CD58, CD79B, CDKN2A, CREBBP, EZH2, EP300, FOXO1, GNA13, HIST1H1B, HIST1H1C, HIST1H1E, KMT2D, KRAS, MEF2B, MYC, MYD88 (273P), PDCD1LG2 (PDL2), PIM1, POU2F2, PRDM1, SOCS1, STAT6, TNFAIP3, and/or TNFRSF14. In some embodiments, the subject has at least one mutation in in one or more sequences encoding: EZH2, MYD88, STAT6A, SOCS1, MYC, and/or HIST1H1E,

In some embodiments, the subject has at least one mutation that decreases or abolishes the function of a gene product (e.g., a transcript, mRNA, or protein) encoded by the mutated sequence as compared to the function of the respective gene product encoded by the wild-type sequence. Such mutations are also sometimes referred to as loss-of-function mutations. Many loss-of-function mutations for the genes and gene products referred to herein that are suitable for some embodiments of this disclosure will be known to the skilled artisan. For example, in some exemplary embodiments, the subject has a loss-of-function mutation in SOCS1. In some embodiments, the subject has at least one mutation that increases the function of a gene product (e.g., a transcript, mRNA, or protein) encoded by the mutated sequence as compared to the function of the respective gene product encoded by the wild-type sequence. Such mutations are also sometimes referred to as gain-of-function mutations or activating mutations. Many gain-of-function mutations for the genes and gene products referred to herein that are suitable for some embodiments of this disclosure will be known to the skilled artisan. For example, in some embodiments, the subject has a gain-of-function mutation in a sequence encoding EZH2, MYD88, STAT6, or MYC. In some embodiments, the subject has at least one loss-of-function and at least one gain-of function mutation. For example, in some embodiments, the subject has at least one gain-of-function mutation in a sequence encoding EZH2 or STAT6, and at least one loss-of-function mutation in a sequence encoding SOCS1. In some embodiments, the subject does not have a specific mutation, e.g., a gain-of-function in a sequence encoding MYC or a loss-of-function mutation in SOCS1.

In some embodiments, the subject expresses a mutant EZH2 protein. In some embodiments, the mutant EZH2 protein comprises a substitution of any amino acid other than tyrosine (Y) for tyrosine (Y) at position 641 of SEQ ID NO: 1, a substitution of any amino acid other than alanine (A) for alanine (A) at position 682 of SEQ ID NO: 1, and/or a substitution of any amino acid other than alanine (A) for alanine (A) at position 692 of SEQ ID NO: 1. In some embodiments, the subject expresses at least one mutant MYD88, STAT6, and/or a SOCS1 protein, either in addition to the mutant EZH2 protein or in the absence of a mutant EZH2 protein. In some embodiments, the subject does not express a mutant MYC and/or a mutant HIST1H1E protein. In some embodiments, the mutant EZH2 protein, the mutant MYD88 protein, the mutant STAT6 protein, and/or the mutant MYC protein exhibits an increase in activity as compared to the respective wild-type protein. In some embodiments, the mutant SOCS1 protein exhibits a decreased activity as compared to the respective wild-type SOCS1 protein.

In some embodiments, the methods provided herein further comprise detecting the at least one mutation in the subject. Such detecting may, in some embodiments, comprise subjecting a sample obtained from the subject to a suitable sequence analysis assay, e.g., to a next generation sequencing assay. Suitable sequencing assays are provided herein or otherwise known to those of skill in the art, and the disclosure is not limited in this respect.

Some aspects of this disclosure provide methods comprising selecting a subject having cancer for treatment with an EZH2 inhibitor based on the presence of at least one mutation associated with a positive response to such treatment in the subject and/or based on the absence of at least one mutation associated with no response or with a negative response to such treatment in the subject. In some embodiments, the at least one mutation associated with a positive response comprises (a) an EZH2 mutation (e.g., a gain-of-function EZH2 mutation); (b) a histone acetyl transferase (HAT) mutation; (c) a STAT6 mutation (e.g., a gain-of-function STAT6 mutation); (d) a MYD88 mutation (e.g., a gain-of-function MYD88 mutation); and/or (e) a SOCS1 mutation (e.g., a loss-of-function SOCS1 mutation). In some embodiments, the at least one mutation associated with no response or with a negative response comprises (a) a MYC mutation (e.g., a gain-of-function MYC mutation); and/or (b) a HIST1H1E mutation. In some embodiments, the method comprises detecting the at least one mutation associated with a positive response and/or the at least one mutation associated with no response or a negative response in a sample obtained from the subject by subjecting the sample to a suitable sequence analysis assay. In some embodiments, the method comprises selecting the subject for treatment with the EZH2 inhibitor based on the subject (a) having at least one of a MYD88 mutation, a STAT6A mutation, and a SOCS1 mutation, and/or (b) not having at least one of a MYC mutation and/or a HIST1H1E mutation. In some embodiments, the method comprises selecting the subject for treatment with the EZH2 inhibitor based on the subject (a) having at least one of a MYD88 mutation, a STAT6A mutation, and a SOCS1 mutation, and (b) not having a MYC mutation and a HIST1H1E mutation.

Some aspects of this disclosure provide methods for selecting a subject having cancer for treatment with an EZH2 inhibitor based on the presence of a mutation profile in the subject that matches a mutation profile (e.g., at least 2, at least 3, at least 4, or at least 5, or more mutations, or, in some embodiments, all mutations), of a patient exhibiting a complete or partial response or stable disease as described in any of FIGS. 1-3.

### Definitions

According to the methods of the disclosure, a “normal” cell may be used as a basis of comparison for one or more characteristics of a cancer cell, including the presence of one or more mutations in a histone acetyltransferase that result in a decreased activity of the enzyme. For example, the one or more mutations in a histone acetyltransferase may result in a decreased acetylation activity or efficacy of the enzyme, and, consequently, a reduced or decreased level of acetylation of at least one lysine on Histone 3 (H3). In certain embodiments, the one or more mutations in a histone acetyltransferase may result in a decreased acetylation activity or efficacy of the enzyme, and, consequently, a reduced or decreased level of acetylation of lysine 27 on Histone 3 (H3) (H3K27). As used herein, a “normal cell” is a cell that cannot be classified as part of a “cell proliferative disorder”. A normal cell lacks unregulated or abnormal growth, or both, that can lead to the development of an unwanted condition or disease. Preferably, a normal cell expresses a comparable amount of EZH2 as a cancer cell. Preferably a normal cell contains a wild type sequence for all histone acetyltransferases, expresses a histone acetyltransferase transcript without mutations, and expresses a histone acetyltransferase protein without mutations that retains all functions a normal activity levels.

As used herein, “contacting a cell” refers to a condition in which a compound or other composition of matter is in direct contact with a cell, or is close enough to induce a desired biological effect in a cell.

As used herein, “treating” or “treat” describes the management and care of a subject for the purpose of combating a disease, condition, or disorder and includes the administration of an EZH2 inhibitor of the disclosure, or a pharmaceutically acceptable salt, prodrug, metabolite, polymorph or solvate thereof, to alleviate the symptoms or complications of cancer or to eliminate the cancer.

As used herein, the term “alleviate” is meant to describe a process by which the severity of a sign or symptom of cancer is decreased. Importantly, a sign or symptom can be alleviated without being eliminated. In a preferred embodiment, the administration of pharmaceutical compositions of the disclosure leads to the elimination of a sign or symptom, however, elimination is not required. Effective dosages are expected to decrease the severity of a sign or symptom. For instance, a sign or symptom of a disorder such as cancer, which can occur in multiple locations, is alleviated if the severity of the cancer is decreased within at least one of multiple locations.

As used herein, the term “severity” is meant to describe the potential of cancer to transform from a precancerous, or benign, state into a malignant state. Alternatively, or in addition, severity is meant to describe a cancer stage, for example, according to the TNM system (accepted by the International Union Against Cancer (UICC) and the American Joint Committee on Cancer (AJCC)) or by other art-recognized methods. Cancer stage refers to the extent or severity of the cancer, based on factors such as the location of the primary tumor, tumor size, number of tumors, and lymph node involvement (spread of cancer into lymph nodes). Alternatively, or in addition, severity is meant to describe the tumor grade by art-recognized methods (see, National Cancer Institute, www.cancer.gov). Tumor grade is a system used to classify cancer cells in terms of how abnormal they look under a microscope and how quickly the tumor is likely to grow and spread. Many factors are considered when determining tumor grade, including the structure and growth pattern of the cells. The specific factors used to determine tumor grade vary with each type of cancer. Severity also describes a histologic grade, also called differentiation, which refers to how much the tumor cells resemble normal cells of the same tissue type (see, National Cancer Institute, www.cancer.gov). Furthermore, severity describes a nuclear grade, which refers to the size and shape of the nucleus in tumor cells and the percentage of tumor cells that are dividing (see, National Cancer Institute, www.cancer.gov).

In another aspect of the disclosure, severity describes the degree to which a tumor has secreted growth factors, degraded the extracellular matrix, become vascularized, lost adhesion to juxtaposed tissues, or metastasized. Moreover, severity describes the number of locations to which a primary tumor has metastasized. Finally, severity includes the difficulty of treating tumors of varying types and locations. For example, inoperable tumors, those cancers which have greater access to multiple body systems (hematological and immunological tumors), and those which are the most resistant to traditional treatments are considered most severe. In these situations, prolonging the life expectancy of the subject and/or reducing pain, decreasing the proportion of cancerous cells or restricting cells to one system, and improving cancer stage/tumor grade/histological grade/nuclear grade are considered alleviating a sign or symptom of the cancer.

As used herein the term “symptom” is defined as an indication of disease, illness, injury, or that something is not right in the body. Symptoms are felt or noticed by the individual experiencing the symptom, but may not easily be noticed by others. Others are defined as non-health-care professionals.

As used herein the term “sign” is also defined as an indication that something is not right in the body. But signs are defined as things that can be seen by a doctor, nurse, or other health care professional.

Cancer is a group of diseases that may cause almost any sign or symptom. The signs and symptoms will depend on where the cancer is, the size of the cancer, and how much it affects the nearby organs or structures. If a cancer spreads (metastasizes), then symptoms may appear in different parts of the body.

As a cancer grows, it begins to push on nearby organs, blood vessels, and nerves. This pressure creates some of the signs and symptoms of cancer. Cancers may form in places where it does not cause any symptoms until the cancer has grown quite large.

Cancer may also cause symptoms such as fever, fatigue, or weight loss. This may be because cancer cells use up much of the body’s energy supply or release substances that change the body’s metabolism. Or the cancer may cause the immune system to react in ways that produce these symptoms. While the signs and symptoms listed above are the more common ones seen with cancer, there are many others that are less common and are not listed here. However, all art-recognized signs and symptoms of cancer are contemplated and encompassed by the disclosure.

Treating cancer may result in a reduction in size of a tumor. A reduction in size of a tumor may also be referred to as “tumor regression”. Preferably, after treatment according to the methods of the disclosure, tumor size is reduced by 5% or greater relative to its size prior to treatment; more preferably, tumor size is reduced by 10% or greater; more preferably, reduced by 20% or greater; more preferably, reduced by 30% or greater; more preferably, reduced by 40% or greater; even more preferably, reduced by 50% or greater; and most preferably, reduced by greater than 75% or greater. Size of a tumor may be measured by any reproducible means of measurement. The size of a tumor may be measured as a diameter of the tumor.

Treating cancer may result in a reduction in tumor volume. Preferably, after treatment according to the methods of the disclosure, tumor volume is reduced by 5% or greater relative to its size prior to treatment; more preferably, tumor volume is reduced by 10% or greater; more preferably, reduced by 20% or greater; more preferably, reduced by 30% or greater; more preferably, reduced by 40% or greater; even more preferably, reduced by 50% or greater; and most preferably, reduced by greater than 75% or greater. Tumor volume may be measured by any reproducible means of measurement.

Treating cancer may result in a decrease in number of tumors. Preferably, after treatment, tumor number is reduced by 5% or greater relative to number prior to treatment; more preferably, tumor number is reduced by 10% or greater; more preferably, reduced by 20% or greater; more preferably, reduced by 30% or greater; more preferably, reduced by 40% or greater; even more preferably, reduced by 50% or greater; and most preferably, reduced by greater than 75%. Number of tumors may be measured by any reproducible means of measurement. The number of tumors may be measured by counting tumors visible to the naked eye or at a specified magnification. Preferably, the specified magnification is 2×, 3×, 4×, 5×, 10×, or 50×.

Treating cancer may result in a decrease in number of metastatic lesions in other tissues or organs distant from the primary tumor site. Preferably, after treatment according to the methods of the disclosure, the number of metastatic lesions is reduced by 5% or greater relative to number prior to treatment; more preferably, the number of metastatic lesions is reduced by 10% or greater; more preferably, reduced by 20% or greater; more preferably, reduced by 30% or greater; more preferably, reduced by 40% or greater; even more preferably, reduced by 50% or greater; and most preferably, reduced by greater than 75%. The number of metastatic lesions may be measured by any reproducible means of measurement. The number of metastatic lesions may be measured by counting metastatic lesions visible to the naked eye or at a specified magnification. Preferably, the specified magnification is 2×, 3×, 4×, 5×, 10×, or 50×.

An effective amount of an EZH2 inhibitor of the disclosure, or a pharmaceutically acceptable salt, prodrug, metabolite, polymorph or solvate thereof, is not significantly cytotoxic to normal cells. For example, a therapeutically effective amount of an EZH2 inhibitor of the disclosure is not significantly cytotoxic to normal cells if administration of the EZH2 inhibitor of the disclosure in a therapeutically effective amount does not induce cell death in greater than 10% of normal cells. A therapeutically effective amount of an EZH2 inhibitor of the disclosure does not significantly affect the viability of normal cells if administration of the compound in a therapeutically effective amount does not induce cell death in greater than 10% of normal cells.

Contacting a cell with an EZH2 inhibitor of the disclosure, or a pharmaceutically acceptable salt, prodrug, metabolite, polymorph or solvate thereof, can inhibit EZH2 activity selectively in cancer cells. Administering to a subject in need thereof an EZH2 inhibitor of the disclosure, or a pharmaceutically acceptable salt, prodrug, metabolite, polymorph or solvate thereof, can inhibit EZH2 activity selectively in cancer cells.

### EZH2 Inhibitors

EZH2 inhibitors of the disclosure comprise tazemetostat (EPZ-6438):

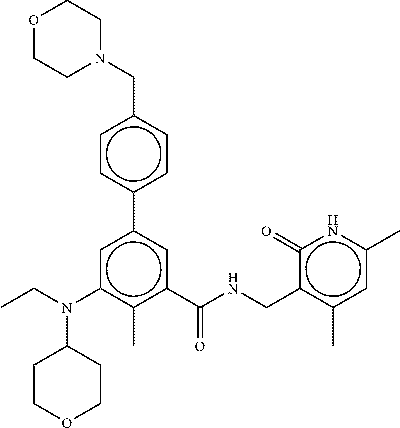

or a pharmaceutically acceptable salt thereof.

Tazemetostat is also described in U.S. Pat. Nos. 8,410,088, 8,765,732, and 9,090,562 (the contents of which are each incorporated herein in their entireties).

Tazemetostat or a pharmaceutically acceptable salt thereof, as described herein, is potent in targeting both WT and mutant EZH2. Tazemetostat is orally bioavailable and has high selectivity to EZH2 compared with other histone methyltransferases (i.e., >20,000 fold selectivity by Ki). Importantly, tazemetostat has targeted methyl mark inhibition that results in the killing of genetically defined cancer cells in vitro. Animal models have also shown sustained in vivo efficacy following inhibition of the target methyl mark. Clinical trial results described herein also demonstrate the safety and efficacy of tazemetostat.

In some embodiments, tazemetostat or a pharmaceutically acceptable salt thereof is administered to the subject at a dose of approximately 100 mg to approximately 3200 mg daily, such as about 100 mg BID to about 1600 mg BID (e.g., 100 mg BID, 200 mg BID, 400 mg BID, 800 mg BID, or 1600 mg BID), for treating a NHL. On one embodiment the dose is 800 mg BID.

EZH2 inhibitors of the disclosure may comprise, consist essentially of or consist of:

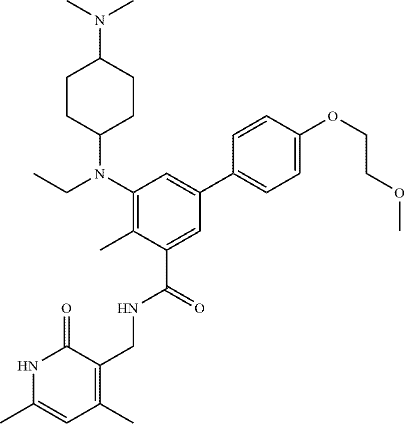

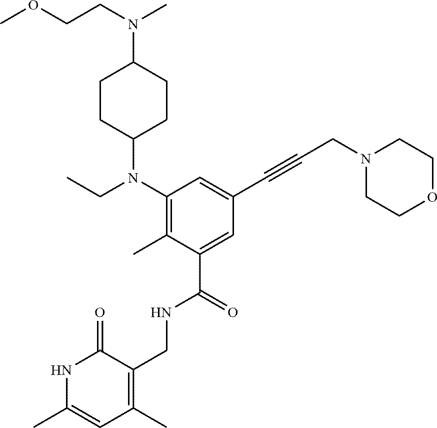 [SMILES: CCN(c1cc(C#CCN2CCOCC2)cc(C(=O)NCc2c(C)cc(C)[nH]c2=O)c1C)C1CCC(N(C)CCOC)CC1]

or

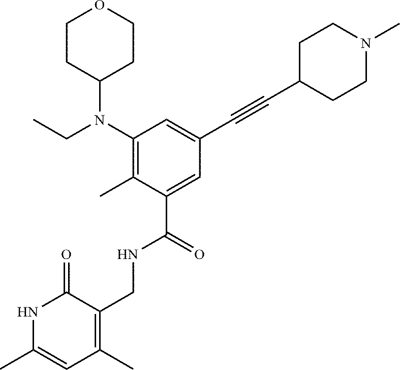

or

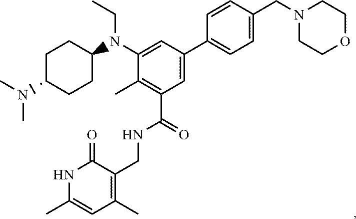

or stereoisomers thereof or pharmaceutically acceptable salts and solvates thereof.

EZH2 inhibitors of the disclosure may comprise, consist essentially of or consist of Compound E:

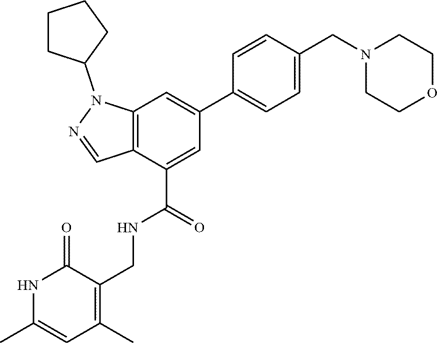 [SMILES: Cc1cc(C)c(CNC(=O)c2cc(-c3ccc(CN4CCOCC4)cc3)cc3c2cnn3C2CCCC2)c(=O)[nH]1]

or pharmaceutically acceptable salts thereof.

EZH2 inhibitors of the disclosure may comprise, consist essentially of or consist of GSK-126, having the following formula:

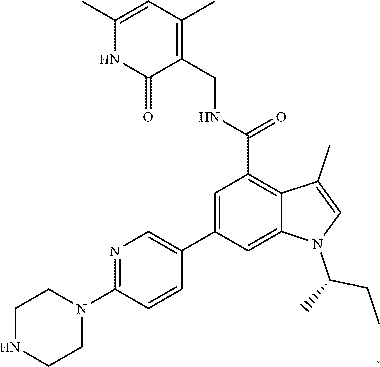

stereoisomers thereof, or pharmaceutically acceptable salts or solvates thereof.

EZH2 inhibitors of the disclosure may comprise, consist essentially of or consist of Compound F:

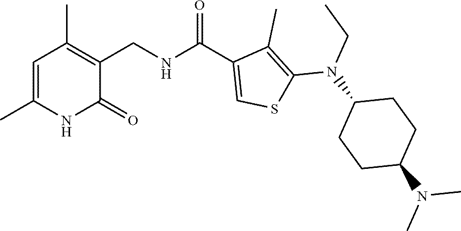

or stereoisomers thereof or pharmaceutically acceptable salts and solvates thereof.

EZH2 inhibitors of the disclosure may comprise, consist essentially of or consist of any one of Compounds Ga-Gc:

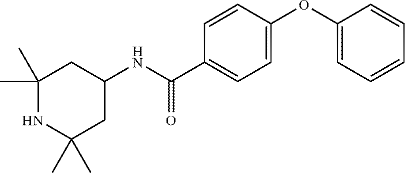 [SMILES: CC1(C)CC(NC(=O)c2ccc(Oc3ccccc3)cc2)CC(C)(C)N1]

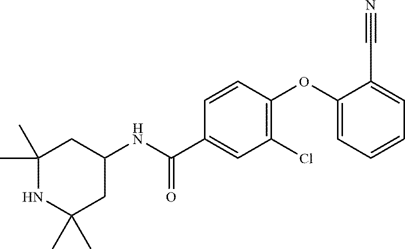 [SMILES: CC1(C)CC(NC(=O)c2ccc(Oc3ccccc3C#N)c(Cl)c2)CC(C)(C)N1]

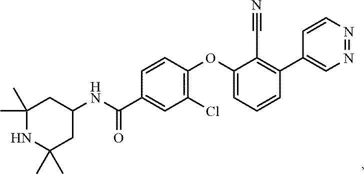

or a stereoisomer, pharmaceutically acceptable salt or solvate thereof.

EZH2 inhibitors of the disclosure may comprise, consist essentially of or consist of CPI-1205 or GSK343.

Additional suitable EZH2 inhibitors will be apparent to those skilled in the art. In some embodiments of the strategies, treatment modalities, methods, combinations, and compositions provided herein, the EZH2 inhibitor is an EZH2 inhibitor described in US 8,536,179 (describing GSK-126 among other compounds and corresponding to WO 2011/140324), the entire contents of each of which are incorporated herein by reference.

In some embodiments of the strategies, treatment modalities, methods, combinations, and compositions provided herein, the EZH2 inhibitor is an EZH2 inhibitor described in PCT/US2014/015706, published as WO 2014/124418, in PCT/US2013/025639, published as WO 2013/120104, and in US 14/839,273, published as US 2015/0368229, the entire contents of each of which are incorporated herein by reference.

In some embodiments, the compound disclosed herein is the compound itself, i.e., the free base or “naked” molecule. In some embodiments, the compound is a salt thereof, e.g., a mono-HCl or tri-HCl salt, mono-HBr or tri-HBr salt of the naked molecule.

Compounds disclosed herein that contain nitrogens can be converted to N-oxides by treatment with an oxidizing agent (e.g., 3-chloroperoxybenzoic acid (mCPBA) and/or hydrogen peroxides) to afford other compounds suitable for any methods disclosed herein. Thus, all shown and claimed nitrogen-containing compounds are considered, when allowed by valency and structure, to include both the compound as shown and its N-oxide derivative (which can be designated as N□O or N⁺-O⁻). Furthermore, in other instances, the nitrogens in the compounds disclosed herein can be converted to N-hydroxy or N-alkoxy compounds. For example, N-hydroxy compounds can be prepared by oxidation of the parent amine by an oxidizing agent such as m-CPBA. All shown and claimed nitrogen-containing compounds are also considered, when allowed by valency and structure, to cover both the compound as shown and its N-hydroxy (i.e., N-OH) and N-alkoxy (i.e., N-OR, wherein R is substituted or unsubstituted C₁-C₆ alkyl, C₁-C₆ alkenyl, C₁-C₆ alkynyl, 3-14-membered carbocycle or 3-14-membered heterocycle) derivatives.

“Isomerism” means compounds that have identical molecular formulae but differ in the sequence of bonding of their atoms or in the arrangement of their atoms in space. Isomers that differ in the arrangement of their atoms in space are termed “stereoisomers.” Stereoisomers that are not mirror images of one another are termed “diastereoisomers,” and stereoisomers that are non-superimposable mirror images of each other are termed “enantiomers” or sometimes optical isomers. A mixture containing equal amounts of individual enantiomeric forms of opposite chirality is termed a “racemic mixture.”

A carbon atom bonded to four nonidentical substituents is termed a “chiral center.”

“Chiral isomer” means a compound with at least one chiral center. Compounds with more than one chiral center may exist either as an individual diastereomer or as a mixture of diastereomers, termed “diastereomeric mixture.” When one chiral center is present, a stereoisomer may be characterized by the absolute configuration (R or S) of that chiral center. Absolute configuration refers to the arrangement in space of the substituents attached to the chiral center. The substituents attached to the chiral center under consideration are ranked in accordance with the Sequence Rule of Cahn, Ingold and Prelog. (Cahn et al., Angew. Chem. Inter. Edit. 1966, 5, 385; errata 511; Cahn et al., Angew. Chem. 1966, 78, 413; Cahn and Ingold, J. Chem. Soc. 1951 (London), 612; Cahn et al., Experientia 1956, 12, 81; Cahn, J. Chem. Educ. 1964, 41, 116).

“Geometric isomer” means the diastereomers that owe their existence to hindered rotation about double bonds or a cycloalkyl linker (e.g., 1, 3-cylcobutyl). These configurations are differentiated in their names by the prefixes cis and trans, or Z and E, which indicate that the groups are on the same or opposite side of the double bond in the molecule according to the Cahn-Ingold-Prelog rules.

It is to be understood that the compounds disclosed herein may be depicted as different chiral isomers or geometric isomers. It should also be understood that when compounds have chiral isomeric or geometric isomeric forms, all isomeric forms are intended to be included in the scope of the disclosure, and the naming of the compounds does not exclude any isomeric forms.

Furthermore, the structures and other compounds discussed in this disclosure include all atropic isomers thereof. “Atropic isomers” are a type of stereoisomer in which the atoms of two isomers are arranged differently in space. Atropic isomers owe their existence to a restricted rotation caused by hindrance of rotation of large groups about a central bond. Such atropic isomers typically exist as a mixture, however as a result of recent advances in chromatography techniques; it has been possible to separate mixtures of two atropic isomers in select cases.

“Tautomer” is one of two or more structural isomers that exist in equilibrium and is readily converted from one isomeric form to another. This conversion results in the formal migration of a hydrogen atom accompanied by a switch of adjacent conjugated double bonds. Tautomers exist as a mixture of a tautomeric set in solution. In solutions where tautomerization is possible, a chemical equilibrium of the tautomers will be reached. The exact ratio of the tautomers depends on several factors, including temperature, solvent and pH. The concept of tautomers that are interconvertible by tautomerization is called tautomerism.

Of the various types of tautomerism that are possible, two are commonly observed. In keto-enol tautomerism a simultaneous shift of electrons and a hydrogen atom occurs. Ring-chain tautomerism arises as a result of the aldehyde group (-CHO) in a sugar chain molecule reacting with one of the hydroxy groups (-OH) in the same molecule to give it a cyclic (ring-shaped) form as exhibited by glucose.

Common tautomeric pairs are: ketone-enol, amide-nitrile, lactam-lactim, amide-imidic acid tautomerism in heterocyclic rings (e.g., in nucleobases such as guanine, thymine and cytosine), imine-enamine and enamine-enamine. An example of keto-enol equilibria is between pyridin-2(1H)-ones and the corresponding pyridin-2-ols, as shown below.

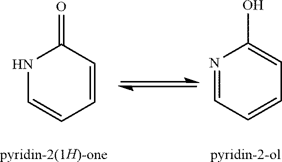

It is to be understood that the compounds disclosed herein may be depicted as different tautomers. It should also be understood that when compounds have tautomeric forms, all tautomeric forms are intended to be included in the scope of the disclosure, and the naming of the compounds does not exclude any tautomer form.

The compounds disclosed herein include the compounds themselves, as well as their salts and their solvates, if applicable. A salt, for example, can be formed between an anion and a positively charged group (e.g., amino) on an aryl- or heteroaryl-substituted benzene compound. Suitable anions include chloride, bromide, iodide, sulfate, bisulfate, sulfamate, nitrate, phosphate, citrate, methanesulfonate, trifluoroacetate, glutamate, glucuronate, glutarate, malate, maleate, succinate, fumarate, tartrate, tosylate, salicylate, lactate, naphthalenesulfonate, and acetate (e.g., trifluoroacetate). The term “pharmaceutically acceptable anion” refers to an anion suitable for forming a pharmaceutically acceptable salt. Likewise, a salt can also be formed between a cation and a negatively charged group (e.g., carboxylate) on an aryl- or heteroaryl-substituted benzene compound. Suitable cations include sodium ion, potassium ion, magnesium ion, calcium ion, and an ammonium cation such as tetramethylammonium ion. The aryl- or heteroaryl-substituted benzene compounds also include those salts containing quaternary nitrogen atoms. In the salt form, it is understood that the ratio of the compound to the cation or anion of the salt can be 1:1, or any ration other than 1:1, e.g., 3:1, 2:1, 1:2, or 1:3.

Additionally, the compounds disclosed herein, for example, the salts of the compounds, can exist in either hydrated or unhydrated (the anhydrous) form or as solvates with other solvent molecules. Nonlimiting examples of hydrates include monohydrates, dihydrates, etc. Nonlimiting examples of solvates include ethanol solvates, acetone solvates, etc.

“Solvate” means solvent addition forms that contain either stoichiometric or non-stoichiometric amounts of solvent. Some compounds have a tendency to trap a fixed molar ratio of solvent molecules in the crystalline solid state, thus forming a solvate. If the solvent is water the solvate formed is a hydrate; and if the solvent is alcohol, the solvate formed is an alcoholate. Hydrates are formed by the combination of one or more molecules of water with one molecule of the substance in which the water retains its molecular state as H₂O.

As used herein, the term “analog” refers to a chemical compound that is structurally similar to another but differs slightly in composition (as in the replacement of one atom by an atom of a different element or in the presence of a particular functional group, or the replacement of one functional group by another functional group). Thus, an analog is a compound that is similar or comparable in function and appearance, but not in structure or origin to the reference compound.

As defined herein, the term “derivative” refers to compounds that have a common core structure, and are substituted with various groups as described herein. For example, all of the compounds represented by Formula (I) are aryl- or heteroaryl-substituted benzene compounds, and have Formula (I) as a common core.

The term “bioisostere” refers to a compound resulting from the exchange of an atom or of a group of atoms with another, broadly similar, atom or group of atoms. The objective of a bioisosteric replacement is to create a new compound with similar biological properties to the parent compound. The bioisosteric replacement may be physicochemically or topologically based. Examples of carboxylic acid bioisosteres include, but are not limited to, acyl sulfonimides, tetrazoles, sulfonates and phosphonates. See, e.g., Patani and LaVoie, Chem. Rev. 96, 3147-3176, 1996.

The present disclosure is intended to include all isotopes of atoms occurring in the present compounds. Isotopes include those atoms having the same atomic number but different mass numbers. By way of general example and without limitation, isotopes of hydrogen include tritium and deuterium, and isotopes of carbon include C-13 and C-14.

### Pharmaceutical Formulations

The present disclosure also provides pharmaceutical compositions comprising at least one EZH2 inhibitor described herein in combination with at least one pharmaceutically acceptable excipient or carrier.

A “pharmaceutical composition” is a formulation containing the EZH2 inhibitors of the present disclosure in a form suitable for administration to a subject. In some embodiments, the pharmaceutical composition is in bulk or in unit dosage form. The unit dosage form is any of a variety of forms, including, for example, a capsule, an IV bag, a tablet, a single pump on an aerosol inhaler or a vial. The quantity of active ingredient (e.g., a formulation of the disclosed compound or salt, hydrate, solvate or isomer thereof) in a unit dose of composition is an effective amount and is varied according to the particular treatment involved. One skilled in the art will appreciate that it is sometimes necessary to make routine variations to the dosage depending on the age and condition of the patient. The dosage will also depend on the route of administration. A variety of routes are contemplated, including oral, pulmonary, rectal, parenteral, transdermal, subcutaneous, intravenous, intramuscular, intraperitoneal, inhalational, buccal, sublingual, intrapleural, intrathecal, intranasal, and the like. Dosage forms for the topical or transdermal administration of a compound of this disclosure include powders, sprays, ointments, pastes, creams, lotions, gels, solutions, patches and inhalants. In some embodiments, the active compound is mixed under sterile conditions with a pharmaceutically acceptable carrier, and with any preservatives, buffers or propellants that are required.

As used herein, the phrase “pharmaceutically acceptable” refers to those compounds, materials, compositions, carriers, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

“Pharmaceutically acceptable excipient” means an excipient that is useful in preparing a pharmaceutical composition that is generally safe, non-toxic and neither biologically nor otherwise undesirable, and includes excipient that is acceptable for veterinary use as well as human pharmaceutical use. A “pharmaceutically acceptable excipient” as used in the disclosure includes both one and more than one such excipient.

A pharmaceutical composition of the disclosure is formulated to be compatible with its intended route of administration. Examples of routes of administration include parenteral, e.g., intravenous, intradermal, subcutaneous, oral (e.g., inhalation), transdermal (topical), and transmucosal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates, and agents for the adjustment of tonicity such as sodium chloride or dextrose. The pH can be adjusted with acids or bases, such as hydrochloric acid or sodium hydroxide. The parenteral preparation can be enclosed in ampoules, disposable syringes or multiple dose vials made of glass or plastic.

A compound or pharmaceutical composition of the disclosure can be administered to a subject in many of the well-known methods currently used for chemotherapeutic treatment. For example, for treatment of cancers, a compound of the disclosure may be injected directly into tumors, injected into the blood stream or body cavities or taken orally or applied through the skin with patches. The dose chosen should be sufficient to constitute effective treatment but not as high as to cause unacceptable side effects. The state of the disease condition (e.g., cancer, precancer, and the like) and the health of the patient should preferably be closely monitored during and for a reasonable period after treatment.

The term “therapeutically effective amount”, as used herein, refers to an amount of an EZH2 inhibitor, composition, or pharmaceutical composition thereof effective to treat, ameliorate, or prevent an identified disease or condition, or to exhibit a detectable therapeutic or inhibitory effect. The effect can be detected by any assay method known in the art. The precise effective amount for a subject will depend upon the subject’s body weight, size, and health; the nature and extent of the condition; and the therapeutic or combination of therapeutics selected for administration. Therapeutically effective amounts for a given situation can be determined by routine experimentation that is within the skill and judgment of the clinician. In a preferred aspect, the disease or condition to be treated is cancer, including but not limited to, B cell lymphoma, including activated B-cell (ABC) and germinal B-cell (GBC) subtypes.

For any EZH2 inhibitor of the disclosure, the therapeutically effective amount can be estimated initially either in cell culture assays, e.g., of neoplastic cells, or in animal models, usually rats, mice, rabbits, dogs, or pigs. The animal model may also be used to determine the appropriate concentration range and route of administration. Such information can then be used to determine useful doses and routes for administration in humans. Therapeutic/prophylactic efficacy and toxicity may be determined by standard pharmaceutical procedures in cell cultures or experimental animals, e.g., ED₅₀ (the dose therapeutically effective in 50% of the population) and LD₅₀ (the dose lethal to 50% of the population). The dose ratio between toxic and therapeutic effects is the therapeutic index, and it can be expressed as the ratio, LD₅₀/ED₅₀. Pharmaceutical compositions that exhibit large therapeutic indices are preferred. The dosage may vary within this range depending upon the dosage form employed, sensitivity of the patient, and the route of administration.

Dosage and administration are adjusted to provide sufficient levels of the active agent(s) or to maintain the desired effect. Factors which may be taken into account include the severity of the disease state, general health of the subject, age, weight, and gender of the subject, diet, time and frequency of administration, drug combination(s), reaction sensitivities, and tolerance/response to therapy. Long-acting pharmaceutical compositions may be administered every 3 to 4 days, every week, or once every two weeks depending on half-life and clearance rate of the particular formulation.

The pharmaceutical compositions containing an EZH2 inhibitor of the present disclosure may be manufactured in a manner that is generally known, e.g., by means of conventional mixing, dissolving, granulating, dragee-making, levigating, emulsifying, encapsulating, entrapping, or lyophilizing processes. Pharmaceutical compositions may be formulated in a conventional manner using one or more pharmaceutically acceptable carriers comprising excipients and/or auxiliaries that facilitate processing of the active compounds into preparations that can be used pharmaceutically. Of course, the appropriate formulation is dependent upon the route of administration chosen.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). In all cases, the composition must be sterile and should be fluid to the extent that easy syringeability exists. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as mannitol, sorbitol, and sodium chloride in the composition. Prolonged absorption of the injectable compositions can be brought about by including in the composition an agent which delays absorption, for example, aluminum monostearate and gelatin.

Sterile injectable solutions can be prepared by incorporating the active compound in the required amount in an appropriate solvent with one or a combination of ingredients enumerated above, as required, followed by filtered sterilization. Generally, dispersions are prepared by incorporating the active compound into a sterile vehicle that contains a basic dispersion medium and the required other ingredients from those enumerated above. In the case of sterile powders for the preparation of sterile injectable solutions, methods of preparation are vacuum drying and freeze-drying that yields a powder of the active ingredient plus any additional desired ingredient from a previously sterile-filtered solution thereof.

Oral compositions generally include an inert diluent or an edible pharmaceutically acceptable carrier. They can be enclosed in gelatin capsules or compressed into tablets. For the purpose of oral therapeutic administration, the active compound can be incorporated with excipients and used in the form of tablets, troches, or capsules. Oral compositions can also be prepared using a fluid carrier for use as a mouthwash, wherein the compound in the fluid carrier is applied orally and swished and expectorated or swallowed. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

For administration by inhalation, the compounds are delivered in the form of an aerosol spray from pressured container or dispenser, which contains a suitable propellant, e.g., a gas such as carbon dioxide, or a nebulizer.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

The active compounds (e.g., EZH2 inhibitors of the disclosure) can be prepared with pharmaceutically acceptable carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811.

It is especially advantageous to formulate oral or parenteral compositions in dosage unit form for ease of administration and uniformity of dosage. Dosage unit form as used herein refers to physically discrete units suited as unitary dosages for the subject to be treated; each unit containing a predetermined quantity of active compound calculated to produce the desired therapeutic effect in association with the required pharmaceutical carrier. The specification for the dosage unit forms of the disclosure are dictated by and directly dependent on the unique characteristics of the active compound and the particular therapeutic effect to be achieved.

In therapeutic applications, the dosages of the pharmaceutical compositions used in accordance with the disclosure vary depending on the agent, the age, weight, and clinical condition of the recipient patient, and the experience and judgment of the clinician or practitioner administering the therapy, among other factors affecting the selected dosage. Generally, the dose should be sufficient to result in slowing, and preferably regressing, the growth of the tumors and also preferably causing complete regression of the cancer. An effective amount of a pharmaceutical agent is that which provides an objectively identifiable improvement as noted by the clinician or other qualified observer. For example, regression of a tumor in a patient may be measured with reference to the diameter of a tumor. Decrease in the diameter of a tumor indicates regression. Regression is also indicated by failure of tumors to reoccur after treatment has stopped. As used herein, the term “dosage effective manner” refers to amount of an active compound to produce the desired biological effect in a subject or cell.

The pharmaceutical compositions can be included in a container, pack, or dispenser together with instructions for administration.

The compounds of the present disclosure are capable of further forming salts. All of these forms are also contemplated within the scope of the claimed disclosure.

As used herein, “pharmaceutically acceptable salts” refer to derivatives of the compounds of the present disclosure wherein the parent compound is modified by making acid or base salts thereof. Examples of pharmaceutically acceptable salts include, but are not limited to, mineral or organic acid salts of basic residues such as amines, alkali or organic salts of acidic residues such as carboxylic acids, and the like. The pharmaceutically acceptable salts include the conventional non-toxic salts or the quaternary ammonium salts of the parent compound formed, for example, from non-toxic inorganic or organic acids. For example, such conventional non-toxic salts include, but are not limited to, those derived from inorganic and organic acids selected from 2-acetoxybenzoic, 2-hydroxyethane sulfonic, acetic, ascorbic, benzene sulfonic, benzoic, bicarbonic, carbonic, citric, edetic, ethane disulfonic, 1,2-ethane sulfonic, fumaric, glucoheptonic, gluconic, glutamic, glycolic, glycollyarsanilic, hexylresorcinic, hydrabamic, hydrobromic, hydrochloric, hydroiodic, hydroxymaleic, hydroxynaphthoic, isethionic, lactic, lactobionic, lauryl sulfonic, maleic, malic, mandelic, methane sulfonic, napsylic, nitric, oxalic, pamoic, pantothenic, phenylacetic, phosphoric, polygalacturonic, propionic, salicyclic, stearic, subacetic, succinic, sulfamic, sulfanilic, sulfuric, tannic, tartaric, toluene sulfonic, and the commonly occurring amine acids, e.g., glycine, alanine, phenylalanine, arginine, etc.

Other examples of pharmaceutically acceptable salts include hexanoic acid, cyclopentane propionic acid, pyruvic acid, malonic acid, 3-(4-hydroxybenzoyl)benzoic acid, cinnamic acid, 4-chlorobenzenesulfonic acid, 2-naphthalenesulfonic acid, 4-toluenesulfonic acid, camphorsulfonic acid, 4-methylbicyclo-[2.2.2]-oct-2-ene-1-carboxylic acid, 3-phenylpropionic acid, trimethylacetic acid, tertiary butylacetic acid, muconic acid, and the like. The present disclosure also encompasses salts formed when an acidic proton present in the parent compound either is replaced by a metal ion, e.g., an alkali metal ion, an alkaline earth ion, or an aluminum ion; or coordinates with an organic base such as ethanolamine, diethanolamine, triethanolamine, tromethamine, N-methylglucamine, and the like.

It should be understood that all references to pharmaceutically acceptable salts include solvent addition forms (solvates) or crystal forms (polymorphs) as defined herein, of the same salt.

The EZH2 inhibitors of the present disclosure can also be prepared as esters, for example, pharmaceutically acceptable esters. For example, a carboxylic acid function group in a compound can be converted to its corresponding ester, e.g., a methyl, ethyl or other ester. Also, an alcohol group in a compound can be converted to its corresponding ester, e.g., an acetate, propionate or other ester.

The EZH2 inhibitors of the present disclosure can also be prepared as prodrugs, for example, pharmaceutically acceptable prodrugs. The terms “pro-drug” and “prodrug” are used interchangeably herein and refer to any compound which releases an active parent drug in vivo. Since prodrugs are known to enhance numerous desirable qualities of pharmaceuticals (e.g., solubility, bioavailability, manufacturing, etc.), the compounds of the present disclosure can be delivered in prodrug form. Thus, the present disclosure is intended to cover prodrugs of the presently claimed compounds, methods of delivering the same and compositions containing the same. “Prodrugs” are intended to include any covalently bonded carriers that release an active parent drug of the present disclosure in vivo when such prodrug is administered to a subject. Prodrugs in the present disclosure are prepared by modifying functional groups present in the compound in such a way that the modifications are cleaved, either in routine manipulation or in vivo, to the parent compound. Prodrugs include compounds of the present disclosure wherein a hydroxy, amino, sulfhydryl, carboxy or carbonyl group is bonded to any group that may be cleaved in vivo to form a free hydroxyl, free amino, free sulfhydryl, free carboxy or free carbonyl group, respectively.

Examples of prodrugs include, but are not limited to, esters (e.g., acetate, dialkylaminoacetates, formates, phosphates, sulfates and benzoate derivatives) and carbamates (e.g., N,N-dimethylaminocarbonyl) of hydroxy functional groups, esters (e.g., ethyl esters, morpholinoethanol esters) of carboxyl functional groups, N-acyl derivatives (e.g., N-acetyl) N-Mannich bases, Schiff bases and enaminones of amino functional groups, oximes, acetals, ketals and enol esters of ketone and aldehyde functional groups in compounds of the disclosure, and the like, See Bundegaard, H., Design of Prodrugs, p1-92, Elesevier, New York-Oxford (1985).

The EZH2 inhibitors, or pharmaceutically acceptable salts, esters or prodrugs thereof, are administered orally, nasally, transdermally, pulmonary, inhalationally, buccally, sublingually, intraperitoneally, subcutaneously, intramuscularly, intravenously, rectally, intrapleurally, intrathecally and parenterally. In some embodiments, the compound is administered orally. One skilled in the art will recognize the advantages of certain routes of administration.

The dosage regimen utilizing the compounds is selected in accordance with a variety of factors including type, species, age, weight, sex and medical condition of the patient; the severity of the condition to be treated; the route of administration; the renal and hepatic function of the patient; and the particular compound or salt thereof employed. An ordinarily skilled physician or veterinarian can readily determine and prescribe the effective amount of the drug required to prevent, counter or arrest the progress of the condition.

The dosage regimen can be daily administration (e.g., every 24 hours) of a compound of the present disclosure. The dosage regimen can be daily administration for consecutive days, for example, at least two, at least three, at least four, at least five, at least six or at least seven consecutive days. Dosing can be more than one time daily, for example, twice, three times or four times daily (per a 24 hour period). The dosing regimen can be a daily administration followed by at least one day, at least two days, at least three days, at least four days, at least five days, or at least six days, without administration.

Techniques for formulation and administration of the disclosed compounds of the disclosure can be found in Remington: the Science and Practice of Pharmacy, 19^{th} edition, Mack Publishing Co., Easton, PA (1995). In some embodiments, the compounds described herein, and the pharmaceutically acceptable salts thereof, are used in pharmaceutical preparations in combination with a pharmaceutically acceptable carrier or diluent. Suitable pharmaceutically acceptable carriers include inert solid fillers or diluents and sterile aqueous or organic solutions. The compounds will be present in such pharmaceutical compositions in amounts sufficient to provide the desired dosage amount in the range described herein.

Methods of the disclosure for treating cancer including treating a B cell lymphoma, including the activated B-cell (ABC) and germinal B-cell (GBC) subtypes. In preferred embodiments, methods of the disclosure are used to treat a subject having a B cell lymphoma. In certain embodiments, the B cell lymphoma cell and/or the subject are characterized as having one or more mutations in a sequence that encodes a histone acetyltransferase (HAT). B cell lymphoma cells may contain a mutation in a gene that encodes a HAT, a corresponding HAT transcript (or cDNA copy thereof), or a HAT protein that decreases/inhibits an activity of a HAT protein. In preferred embodiments, the mutation in a gene that encodes a HAT, a corresponding HAT transcript (or cDNA copy thereof), or a HAT protein that decreases/inhibits an activity of a HAT protein, decreases or inhibits an acetylation activity or efficacy of the enzyme, resulting in a decreased level of acetylation of one or more lysines of histone 3 (H3) (e.g., H3K27). The presence of the HAT mutation resulting in a decreased level of acetylation of one or more lysines of histone 3 (H3) (e.g., H3K27) in a cell renders that cell sensitive to oncogenic transformation and treatment with an EZH2 inhibitor.

Methods of the disclosure may be used to treat a subject who has one or more mutations in a HAT that decrease/inhibit the ability of the HAT to acetylate one or more lysines of histone 3 (H3) (e.g., H3K27) or who has one or more cells with one or more mutations in a HAT that decrease/inhibit the ability of the HAT to acetylate one or more lysines of histone 3 (H3) (e.g., H3K27). HAT expression and/or HAT function may be evaluated by fluorescent and non-fluorescent immunohistochemistry (IHC) methods, including well known to one of ordinary skill in the art. In a certain embodiment the method comprises: (a) obtaining a biological sample from the subject; (b) contacting the biological sample or a portion thereof with an antibody that specifically binds HAT; and (c) detecting an amount of the antibody that is bound to HAT. Alternatively, or in addition, HAT expression and/or HAT function may be evaluated by a method comprising: (a) obtaining a biological sample from the subject; (b) sequencing at least one DNA sequence encoding a HAT protein from the biological sample or a portion thereof; and (c) determining if the at least one DNA sequence encoding a HAT protein contains a mutation affecting the expression and/or function of the HAT protein. HAT expression or a function of HAT may be evaluated by detecting an amount of the antibody that is bound to HAT and by sequencing at least one DNA sequence encoding a HAT protein, optionally, using the same biological sample from the subject.

All percentages and ratios used herein, unless otherwise indicated, are by weight.

Other features and advantages of the present disclosure are apparent from the different examples. The provided examples illustrate different components and methodology useful in practicing the present disclosure. The examples do not limit the claimed disclosure. Based on the present disclosure the skilled artisan can identify and employ other components and methodology useful for practicing the present disclosure.

### EXAMPLES

In order that the invention disclosed herein may be more efficiently understood, examples are provided below. It should be understood that these examples are for illustrative purposes only and are not to be construed as limiting the disclosure in any manner.

### Example 1: Molecular Predictors of Response to EZH2 Inhibition in NHL Patients

Some B-cell malignancies depend on enhancer of zeste homolog 2 (EZH2; a histone methyl transferase) to perpetuate a less differentiated state, for example, on activating mutations of EZH2 being potential oncogenic drivers.

Tazemetostat is an oral, first-in-class, potent, and highly selective inhibitor of EZH2 currently in phase 2 clinical development for the treatment of relapsed or refractory non-Hodgkin lymphoma (NHL).

In the phase 1 part of a phase ½ study (NCT01897571) of adult patients with advanced solid tumors or B-cell lymphomas, objective responses to tazemetostat were observed in patients with tumors exhibiting mutated or wild-type EZH2.

An ongoing phase 2 study (NCT01897571) is enrolling patients with relapsed or refractory diffuse large B-cell lymphoma (DLBCL) or follicular lymphoma (FL) to assess the efficacy and safety of tazemetostat. Patients with tumors exhibiting mutated or wild-type EZH2 are eligible to participate. The primary study endpoint is objective overall response rate.

A molecular analysis of tumor material obtained in the phase 2 study and its associations with preliminary response data is described herein, including, inter alia, a molecular predictor and a molecular predictor profile for responsiveness of a tumor or subject to treatment with an EZH2 inhibitor, e.g., with tazemetostat.

### Methods & Results

During screening, archive tumor- and/or plasma-derived circulating tumor DNA (ctDNA) samples were obtained. Archive tumor was analyzed prospectively for EZH2-activating mutations Y646F, Y646N, Y646X (Y646H, Y646C, and Y646S), A682G, and A692V using a cobas® EZH2 Mutation Test (Roche Molecular Systems, Inc., Pleasanton, CA, USA). Next generation sequencing (NGS) was performed retrospectively on DNA derived from archive tumor and plasma samples to identify somatic mutations, amplifications, and translocations using an NHL-specific 62-gene panel on the Illumina HiSeq 2500 platform (Illumina, Inc., San Diego, CA, USA) with 100 bp paired-end reads (Table 1).

TABLE 1

Content for a targeted NGS panel representing 62 genes reported to be mutated in >5% DLBCL or FL patients
Custom Lymphoma CancerSelect-R Sequence – Mutation Gene List

| Gene Name | | Gene Name | |
|---|---|---|---|
| PRDM1′ | CD58 | KRAS | PTPRD |
| EZH2′ | CD798 | MEF28 | RB1 |
| KDM6A | CDKN2A | MYC | S1PR2 |
| KMT2D′ | CREBBP | MYD88 | SGK1 |
| ARID1A | EP300 | NOTCH1 | SMARCB1 |
| ATM | FOXO1 | NOTCH2 | SOCS1 |
| B2M | GNA13 | NRAS | STAT6 |
| BCL2 | HIST1H18 | PIK3CA | TBL1XR1 |
| BCL6 | HIST1H1E | PIM1 | TNFAIP3 |
| BCL7A | IKZF3 | POU2F2 | TNFRSF14 |
| BRAF | IRF4 | PTEN | TP53 |
| BTG1 | CD58 | PTPN1 | XPO1 |
| CARD11 | ITPKB | PTPN11 | |
| CCND3 | KIT | PTPN6 | |
| Custom Lymphoma CancerSelect-R Translocation Gene List | | | Customer Lymphoma CancerSelect-R Amplification Gene List |
| Gene Name | Sequence Region(s) included | | Gene Name |
| ALK | ALK_NM_004304_Intron19 | | BCL2 |
| BCL2 | BCL2_MCR_Breakpoint_Region | | CD274 (PDL1) |
| BCL2 | BCL2_MBR_Breakpoint_Region | | PDCD1LG2 |
| BCL6 | Entire Gene | | (PDL2) |
| CIITA | Entire Gene | | FOXP1 |
| MYC | Entire Gene + 40k8P upstream | | IAK2 |
| CD274 (PDL1) | Entire Gene | | KDM4c |
| PDCD1LG2 (PDL2) | Entire Gene | | REL |

*Full coding sequence analyzed. Specific exons were otherwise sequenced for all genes in the mutation list.

Average target coverage for the tissue panel was 1250 X, while coverage for the

ctDNA was approximately 20,000 X for mutations and 3700 X for structural alterations. Sequencing data were aligned to the human reference sequence (hg19) and analyzed using validated cancer genome analysis algorithms (Personal Genome Diagnostics, Baltimore, MD, USA). EZH2 mutation detection rates in archive tumor were ~12% in DLBCL and 19% in FL in the total phase 2 cohort (Table 2), consistent with previous reports.

TABLE 2

Demographics and disease characteristics for phase 2 patient cohort and subset sequenced by NGS

| Characteristic | Phase 2 Total Cohort | NGS Cohort |
|---|---|---|
| n | 204 | 92 |
| Age, median years | 66 | 65 |
| Males, % | 58 | 63 |
| ECOG PS. median (range) | 1 (0-2) | 0 (0-2) |
| Prior lines of therapy, n (%) | | |
| 1-2 | 61 (30) | 26 (28) |
| 3-4 | 83 (41) | 35 (38) |
| ≥ 5 | 60 (29) | 31 (34) |
| median | 3 | 4 |
| Median time from initial diagnosis, years | 2.7 | 3.5 |
| Median time from last prior therapy, weeks | 15.1 | 13.9 |
| Refractory to last regimen, n (%) | 122 (60) | 52 (57) |
| DLBCL n (%) | | |
| EZH2 WT^{a} | 120 (59) | 57 (62) |
| EZH2 mutant^{a} | 17 (8) | 6 (6) |
| FL, n (%) | | |
| EZH2 WT^{a} | 54 (26) | 24 (26) |
| EZH2 Mutant^{a} | 13 (6) | 5 (5) |

^{a}Determined by cobas® EZH2 Mutation Test ECOG PS, Eastern Cooperative Oncology Group performance status; WT, wild-type

Regardless of the technology or sample type used, the concordance rate for detection of EZH2 status was >95%. NGS and cobas® Test of archive tumor samples was 98.9% concordant (n=92) with 11 EZH2 activating mutation cases detected (Table 3). Concordance of EZH2 status between archive tumor and ctDNA samples was 97% (n=125).

TABLE 3

Comparison EZH2 activating mutation detection between cobas® EZH2

| Cohort Designation | Cell of Origin (Nanostring) | EZH2 cobas® Test | Tumor Content for cobas® Test (%) | Archive Tumor NGS Result (vaf) (%) | ctDNA NGS Result (vaf) | Clonal or Subclonal EZH2 Mutation^{a} |
|---|---|---|---|---|---|---|
| GCB-DLBCL EZH2 MT | GCB DLBCL | Y646F | 100 | EZH2 Y646F (10) | EZH2 Y646F (1.3%) | Subclonal |
| Non-GCB DLBCL | GCB DLBCL | Y646X | 20 | EZH2 Y646H (19) | EZH2 Y646H (12.7%) | Clonal |
| Non-GCB DLBCL | Unclassified | Y646F | 50 | EZH2 Y646F (23) | Data pending | Clonal |
| GCB-DLBCL EZH2 MT | GCB DLBCL | Y646F | 100 | EZH2 Y646F (38) | EZH2 Y646F (8.9%) | Clonal |
| FL EZH2 mutant | N/A | Y646F | 100 | EZH2 Y646F (8) | Not detected | Subclonal |
| GCB-DLBCL EZH2 MT | Not performed | A682G | 95 | EZH2 A682G (34) | EZH2 A682G (0.9%) | Clonal |
| FL EZH2 mutant | N/A | Y646N | 90 | EZH2 Y646N (22) | Low DNA yield | Clonal |
| FL EZH2 mutant | N/A | Y646X | 100 | EZH2 Y646S (22) | EZH2 Y646S (6.6%) | Clonal |
| GCB-DLBCL EZH2 MT | GCB DLBCL | Y646X | UK | EZH2 Y646H (25) | EZH2 Y646H (28%) | Clonal |
| FL EZH2 mutant | N/A | Y646X | 100 | EZH2 Y646H (2) | EZH2 Y646H (0.7%) | Subclonal |
| FL EZH2 mutant | N/A | Y646X | 100 | Not detected | Not detected | N/A |

^{a}Patients determined to have EZH2 mutant (MT) tumor DNA copies ≥20% were considered clonal.

Mutations in EZH2, STAT6, or MYD88 in wild-type EZH2 patients were associated with response (P<0.1), whereas mutations in HIST1H1E, TP53, or MYC (P<0.08) were associated with nonresponse (Table 4).

Activating mutations in EZH2 and MYD88 (e.g., MYD88 L273P) were mutually exclusive. Accordingly, MYD88 was identified as a biomarker for response to treatment with EZH2 inhibitors, e.g., with tazemetostat, in wild-type EZH2 patients.

TABLE 4

Variants associated with tazemetostat response using gene∼level analysis of archive tumor Tissue (responders n=25; nonresponders n=36)

| | | | | | | | |
|---|---|---|---|---|---|---|---|
| Gene | TP53 | HIST1H1E | EZH2 | MYC | STAT6 | MYD88 L273P | All Patients |
| Responder, n | 6 | 1 | 7 | 0 | 7 | 4 | 25 |
| Nonresponder, n | 21 | 14 | 2 | 7 | 3 | 2 | 36 |
| P-value | 0.0079 | 0.02 | 0.0253 | 0.0727 | 0.0753 | 0.0841 | NA |
| Response preference | WT | WT | Mutant | WT | Mutant | Mutant | NA |

Activating mutations in EZH2 AND MYD88 are mutually exclusive

Patients matching a multigene predictor consisting of wild-type MYC and/or HIST1H1E but with mutated STAT6 and/or MYD88 in archive tumor had an objective overall response rate of ~58% (11/19), whereas patients who did not match this profile had an objective overall response rate of ∼ 19% (14/73), demonstrating the potential for these four genes to predict response to tazemetostat (Table 5).

TABLE 5

Results of predictive modelling to identify potential Results of predictive modelling to identify potential multigene predictors of, response to archival tumor
Multigene Predictor Performance for Archival Tumor response from NHL Patients

| | Patients Matching Profile n (%) | Patients Not Matching Proflle n (%) |
|---|---|---|
| Genetic Profile 1 | 19 | 73 |
| Responder (CR/PR) | 11 (57.9) | 14(19.2) |
| Nonresponder (SD/PF/UK) | 8 (42.1) | 59 (80.8) |
| Genetic Profile 2 | 26 | 66 |
| Responder (CR/PR) | 15 (57.7) | 10 (15.2) |
| Nonresponder (SD/PD/UK) | 11 (42.3) | 56 (84.8) |

Genetic Profile 1 = MYC wt + HISTIH1E wt + (STAT6/any on MYD88/L273).
Genetic Profile 2 = MYC wt + HISTIH1E wt + (STAT6/any MYD88/L273 or EZH2/Y646, A682, A692).

The molecular genetic profiling of NBL patients described here identifies predictors and predictor profiles for the response to treatment with EZH2 inhibitors, e.g., with tazemetostat. In addition, the results disclosed herein demonstrate that plasma-based ctDNA screening is a viable method to identify NHL patients with mutations, e.g., with EZH2-activating mutations, in the absence of archive tumor samples

All publications and patent documents cited herein are incorporated herein by reference as if each such publication or document was specifically and individually indicated to be incorporated herein by reference. Citation of publications and patent documents is not intended as an admission that any is pertinent prior art, nor does it constitute any admission as to the contents or date of the same. The invention having now been described by way of written description, those of skill in the art will recognize that the invention can be practiced in a variety of embodiments and that the foregoing description and examples below are for purposes of illustration and not limitation of the claims that follow. Where names of cell lines, genes or proteins are used herein, abbreviations and names conform to the nomenclature of the American Type Culture Collection (ATCC) or the National Center for Biotechnology Information (NCBI), unless otherwise noted or evident from the context.

The invention can be embodied in other specific forms without departing from the spirit or essential characteristics thereof. The foregoing embodiments are therefore to be considered in all respects illustrative rather than limiting on the invention described herein. Scope of the invention is thus indicated by the appended claims rather than by the foregoing description, and all changes that come within the meaning and range of equivalency of the claims are intended to be embraced therein.

1. A method, comprising administering a therapeutically effective amount of an inhibitor of Enhancer of Zeste Homolog 2 (EZH2) sufficient to treat a cancer to a subject in need thereof, wherein the cancer is characterized by at least one mutation in one or more sequences encoding a gene or gene product listed in Tables 1-5, and/or FIGS. 1-3.

2-8. (canceled)

9. The method of claim 1, wherein the method further comprises detecting the at least one mutation in the subject.

10. The method of claim 9, wherein the detecting comprises subjecting a sample obtained from the subject to a sequence analysis assay.

11. (canceled)

12. The method of claim 10, wherein the detecting comprises obtaining a sample from the subject.

13. The method of claim 12, wherein the sample is a tumor sample or a sample that comprises a cancer cell.

14. The method of claim 12, wherein the sample is a sample comprising ctDNA.

15. The method of claim 1, wherein the inhibitor of EZH2 is 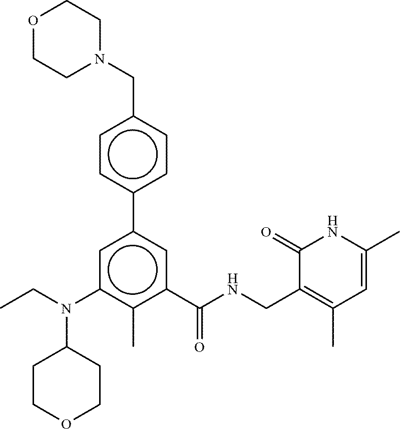
(tazemetostat), or a pharmaceutically-acceptable salt thereof.

16-25. (canceled)

26. The method of claim 1, wherein the at least one mutation comprises a MYD88 and/or STAT6A mutation.

27. The method of claim 1, wherein the cancer does not have a MYC and/or a HIST1H1E mutation.

28. The method of claim 1, wherein the cancer (a) has a MYD88 gain-of-function or a STAT6A loss-of-function mutation, and (b) has neither a MYC nor a HIST1H1E mutation.

29. The method of claim 1, wherein the cancer (a) has a MYD88 L273 mutation or a STAT6A loss-of-function mutation, (b) has neither a MYC nor a HIST1H1E mutation, and (c) does not have an EZH2 gain-of-function mutation.

30-31. (canceled)

32. The method of claim 1, wherein the cancer is B-cell lymphoma.

33-34. (canceled)

35. The method of claim 1, wherein the cancer is follicular lymphoma.

36. A method, comprising selecting a subject having cancer for treatment with an EZH2 inhibitor based on the presence of at least one mutation associated with a positive response to such treatment in the subject and/or based on the absence of at least one mutation associated with no response or with a negative response to such treatment in the subject.

37. The method of claim 36, wherein the at least one mutation associated with a positive response comprise
(a) an EZH2 gain-of-function mutation or a MYD88 gain-of-function mutation; and,
(b) a STAT6 loss-of-function mutation.

38. The method of claim 36, wherein the at least one mutation associated with no response or with a negative response comprise
(a) a MYC mutation;
(b) a TP53 mutation; and/or,
(c) a HIST1H1E mutation.

39. The method of claim 36, wherein the method comprises detecting the at least one mutation associated with a positive response and/or the at least one mutation associated with no response or a negative response in a sample obtained from the subject.

40. The method of claim 36, wherein the method comprises selecting the subject for treatment with the EZH2 inhibitor based on the subject (a) having either an EZH2 or a MYD88 gain-of-function mutation, or a STAT6A loss-of-function mutation; and (b) having neither a MYC mutation, nor a TP53 mutation, nor a HIST1H1E mutation.

41. The method of claim 36, wherein the method comprises selecting the subject for treatment with the EZH2 inhibitor based on the subject
(a) having a MYD88 L273 mutation or a STAT6A mutation; and,
(b) having neither a MYC mutation, nor a TP53 mutation, nor a HIST1H1E mutation.

42-45. (canceled)

46. A method, comprising selecting a subject having cancer for treatment with an EZH2 inhibitor based on the presence of a mutation profile in the subject that matches a mutation profile of a patient exhibiting a complete or partial response or stable disease in any of Tables 1-5 or Figures 1-3.

47-92. (canceled)
